(19) United States
(12) Patent Application Publication
(10) Pub. No.: US 2026/0183283 A1
(43) Pub. Date: Jul. 2, 2026

(54) TREATMENT OF ORGANIC ACIDEMIAS OR PANTOTHENATE KINASE ASSOCIATED NEURODEGENERATION WITH MODULATORS OF PANTOTHENATE KINASES

(71) Applicant: St. Jude Children's Research Hospital, Inc., Memphis, TN (US)

(72) Inventors: Daniel David GRETLER, Palo Alto, CA (US); Agnieszka Sylwia JURECKA, Palo Alto, CA (US); Rajaa SUKHUN, Palo Alto, CA (US)

(21) Appl. No.: 18/868,338

(22) PCT Filed: May 25, 2023
(86) PCT No.: PCT/US2023/067478

§ 371 Date: Nov. 22, 2024

Related U.S. Application Data
(60) Provisional application No. 63/346,058, filed on May 26, 2022.
(60) Provisional application No. 63/346,063, filed on May 26, 2022.

(51) Int. Cl. A61K 31/501; A61P 3/00; A61P 25/28

(52) U.S. Cl. CPC A61K 31/501; A61P 3/00; A61P 25/28

(57) ABSTRACT

The present disclosure provides a method of treating an organic acidemia (e.g., propionic acidemia (PA) and/or methyl-malonic acidemia (MMA)) or Pantothenate Kinase Associated Neurodegeneration (PKAN) in a subject with a therapeutically effective amount of a compound represented by formula (I): a pharmaceutically acceptable salt, hydrate, solvate, or a combination thereof. In particular, the subject has propionic acidemia (PA) and/or methylmalonic acidemia (MMA).

### CROSS-REFERENCES TO RELATED APPLICATIONS

This application claims priority to U.S. Provisional Application No. 63/346,058 filed May 26, 2022 and U.S. Provisional Application No. 63/346,063 filed May 26, 2022, each of which is incorporated herein in its entirety for all purposes.

### STATEMENT AS TO RIGHTS TO INVENTIONS MADE UNDER FEDERALLY SPONSORED RESEARCH AND DEVELOPMENT

Not Applicable

### REFERENCE TO A “SEQUENCE LISTING,” A TABLE, OR A COMPUTER PROGRAM LISTING APPENDIX SUBMITTED ON A COMPACT DISK

The present application contains a Sequence Listing, which is incorporated herein in its entirety for all purposes. The accompanying Sequence Listing is submitted in a file entitled “052650-507001WO.xml”, which was created on May 23, 2023, and is approximately 24,157 bytes in size.

### BACKGROUND

Organic acidemias (OAs) are rare, autosomal recessive, multisystemic inborn errors of branched-chain amino acid metabolism that cause significant morbidity and mortality in infancy and childhood (Fraser and Venditti 2016). OAs are generally characterized by excessive organic acids and toxic metabolites accumulating in the blood and urine (Fenton et al. 2001). There are 3 categories of organic acidemias: 1) systemic OAs (such as propionic acidemia (PA) and methylmalonic acidemia (MMA)), 2) cerebral OAs, and 3) ketolytic/ketogenic OAs (Chapman 2019).

Both PA and MMA result in defective mitochondrial metabolism of CoA-activated carboxylic acids, which are largely derived from the metabolism of branched-chain amino acids, odd-chain fatty acids, and cholesterol (Fraser and Venditti 2016). PA and MMA result in a lack of sufficient succinyl-coenzyme A (CoA) generation to fuel the tricarboxylic acid cycle (TCA cycle), resulting in deficient cellular energy production and leading to the accumulation of toxic metabolites. Accumulation of these toxic metabolites, aberrant mitochondrial energy metabolism, camitine depletion, and CoA sequestration contribute to the pathological features of PA and MMA (Fraser and Venditti 2016).

Propionic acidemia and MMA classically present in a neonate within the first 3 days of life, and a diagnosis of PA and MMA is typically made during the neonatal period and early childhood, often after an episode of acute metabolic decompensation (Kölker et al. 2015). In the United States, diagnosis may occur after standard newborn screening blood tests. Neonates and older children frequently present with symptoms that resemble sepsis (i.e., lethargy, confusion, decreased intake, vomiting, coma, and, if untreated, death) (Chapman 2019). Catabolism is the predominant trigger for metabolic decompensation, as breakdown of amino acids increases levels of toxic metabolites that are acidic in nature and thus decreases the pool of bicarbonate, and is often accompanied by ketoacidosis and hyperammonemia (Haijes et al. 2019). Halting protein catabolism by limiting protein intake and increasing caloric intake is one of very few treatment options for patients with PA and MMA. Patients with PA and MMA are susceptible to a variety of severe complications, which continue to occur even without acute metabolic decompensation (Baumgartner et al. 2014). The most prevalent complications in PA include hepatomegaly and/or a hyperechoic liver, cognitive and psychomotor deficits, anemia, and muscular hypotonia/hypotonicity followed by different degrees of disabilities (Haijes et al. 2019). In MMA, the most prevalent complications include joint hypermobility, pes planus (flat feet), cognitive deficits, enamel defects, and psychomotor retardation (Haijes et al. 2019). End-organ injury in PA and MMA tends to occur in organs with high-energy demands, including the brain, heart, kidney, and eye due to primary toxicity associated with the accumulation of primary and secondary metabolites and decreased succinyl-CoA resulting in dysfunction of the TCA cycle and oxidative phosphorylation resulting in cell energy deficiency (Fraser and Venditti 2016).

There are currently no approved therapies for PA and MMA that target their pathophysiological mechanisms. Standard of care for patients with PA and MMA is largely pre-symptomatic and typically involves proactive adjustment and maintenance on a protein-limited diet and may include treatment with metronidazole to reduce propiogenic precursors. For patients with gastric feeding tubes, a combination of age-appropriate enteral formulas and formulas designed for organic acidemia patients are utilized (Fraser and Venditti 2016). PA patients are often placed on low-protein or synthetic diets to reduce C3-CoA formation.

However, closely following prescribed diets is known to be very difficult. Camitine supplementation (L-carnitine) is often recommended for patients with PA and MMA to prevent secondary carnitine deficiency and B12 responsive MMA patients may be treated with daily hydroxocobalamin injections (Fraser and Venditti 2016). Beyond carnitine supplementation, other forms of medical management are sometimes employed, including antibiotics to reduce intestinal propionate-producing flora, precursor-free amino acid and/or isoleucine/valine supplementation, and vitamin/mineral supplementation (Baumgartner et al. 2014).

In addition to diet control and symptom management, liver, kidney, and combined liver-kidney transplantation are currently suggested as a treatment option for PA and MMA, particularly for patients suffering from recurrent metabolic decompensation (Forny et al. 2021). However, the data on success of transplantation for patients with PA and MMA are uncertain and evolving. While metabolic outcomes can be significantly improved, transplantation is associated with inherent risk from chronic immunosuppression to prevent organ rejection, and the biochemical defects associated with PA and MMA are only partially corrected (Yap et al. 2020). Approximately 15% of patients continue to have episodes of metabolic decompensation post-transplant and, for patients who decrease restrictions on protein intake post-transplant, the risk of metabolic decompensation can be even higher (Yap et al. 2020). This suggests the need for continued metabolic intervention even after transplant. Accordingly, there remains a need for improved therapies for PA and MMA.

Pantothenate Kinase Associated Neurodegeneration (PKAN) is a rare, autosomal recessive disorder caused by mutations in the PANK2 gene which encodes the pantothenate kinase (PanK) 2 protein. PKAN is a debilitating and life-threatening condition characterized by deposition of iron in the basal ganglia and progressive extrapyramidal decline affecting movement, balance, speech, vision, cognition, and behavior. Earlier disease onset, more rapid progression to loss of ambulation, and premature death have been observed in patients with two null alleles for PANK2, although for many patients there is no clear genotype-phenotype correlation. The majority of the mutations associated with PKAN result in the expression of truncated or mutant PanK2 proteins with little or no catalytic activity. There are three closely related mammalian isoforms of PanK that govern intracellular coenzyme A (CoA) levels, PanK1, PanK2, and PanK3. All three isoforms are expressed in the brain, with PanK2 and PanK3 being the dominant central nervous system (CNS) isoforms in humans. PanK is the first and rate-controlling step in the only pathway for CoA biosynthesis. PanK enzymes tightly control CoA production, with the degree of enzymatic activity related to feedback inhibition of acetyl-CoA to stabilize CoA levels. CoA is a major acyl group carrier in biology and participates as a key cofactor and regulator of oxidative metabolism of fatty acids, amino acids, and ketones for energy production and growth. CoA is a critical metabolite required by the body in many mitochondrial and intracellular reactions, such as the citric acid cycle for the metabolism of pyruvate and the synthesis of oxidation of fatty acids. PANK2 mutations result in expression of truncated or mutant PanK2 proteins with little or no enzymatic activity, leading to a deficiency in intracellular CoA in the brain, oxidative stress, membrane damage, cell death, and subsequent neurodegeneration.

PKAN symptoms are thought to arise from a CoA deficiency in the brain that compromises important neuronal processes including iron metabolism, synaptic transmission, synaptic vesicle cycling, neuron projection development, and protein quality control. PKAN is a progressive neurodegenerative disorder affecting movement, balance, speech, vision, cognition, affect, and behavior. It covers a continuous clinical spectrum with 2 major forms generally described in the literature: classic and atypical, based on the age of onset and disease progression. Hallmark features of classic PKAN (early onset with rapid progression) include dystonia onset before the age of 10 years, loss of ambulation within 10 to 15 years of onset, and main presenting features of overlapping dystonia-parkinsonism syndrome, combined with motor disturbances and early disability. In atypical PKAN (later onset with slower progression, including all non-classic phenotypes), dystonia onset is more commonly in the second or third decade of life, loss of ambulation may occur within 15 to 40 years of onset, and main presenting features include parkinsonism, dysarthria, and dystonia. Despite this classification, there are patients with early onset but slow progression or late onset with rapid progression, which are often classified as intermediate. The pattern of PKAN progression is saltatory, with stepwise decline occurring over a few days or weeks, followed by plateauing that may be sustained for weeks, months, or even years. The overall trajectory of change is that of relentless progression. Clinical manifestations of PKAN include developmental delay, focal and generalized dystonia (sometimes causing intractable pain), choreoathetosis, status dystonicus, parkinsonism with Parkinson's-like freezing and bradykinesia, dysarthria, spasticity, rigidity, retinal degeneration (leading to loss of vision), and dysphagia (often leading to feeding tube placement). Early mortality is typically caused by disease-associated sequelae such as malnutrition, aspiration, or pneumonia.

Substituted 1-(4-pyridazin-3-yl)piperazin-1-yl-2-phenylethan-1-one compounds refer to a class of compounds having modulatory activities against pantothenate kinase (PanK) activity, as disclosed in International Patent Application Nos. PCT/US2017/039037 filed Jun. 23, 2017 and PCT/US2018/067539 filed Dec. 26, 2018, each of which is incorporated herein in its entirety for all purposes. In particular, the compounds are represented by the following formula:

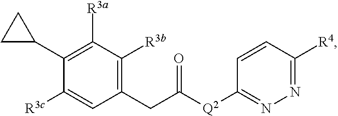

or a pharmaceutically acceptable salt thereof, wherein Q², R^{3a}, R^{3b}, R^{3c}, and R⁴ are as provided in PCT/US2018/067539. More specifically, the substituted 1-(4-pyridazin-3-yl)piperazin-1-yl-2-phenylethan-1-one compound is represented by formula (I):

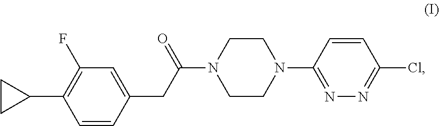 [SMILES: O=C(Cc1ccc(C2CC2)c(F)c1)N1CCN(c2ccc(Cl)nn2)CC1]

or a pharmaceutically acceptable salt, hydrate, solvate, or a combination thereof.

The compound of formula (I) (hereafter as Compound (I)) is an orally bioavailable small molecule that targets the pantothenate kinases, which catalyze the first step and control the rate of CoA biosynthesis. CoA synthesis is the only metabolic fate of pantothenate, also known as pantothenic acid or vitamin B₅, an essential component of the human diet. Compound (I) is posited to activate the PanKs and inhibit the negative feedback of propionyl-CoA on the pantothenate kinase enzymes, thereby increasing the pool of free intracellular CoA in the liver and central nervous system. This increases CoA availability for ligation with metabolic intermediates that are components of many mitochondrial and intracellular reactions, such as the citric acid cycle for the conversion of pyruvate to acetyl-CoA and the synthesis and oxidation of fatty acids. Compound (I) binds selectively and potently to both PanK2 and PanK3 with Kᵢs less than 5 nanomolar (nM). The PanK isoform expression levels and their potent feedback inhibition by acyl-CoAs control and stabilize the intracellular CoA content in cells and tissues. Compound (I) binding renders the PanKs refractory to feedback inhibition by acyl-CoAs, resulting in increased CoA production in both the liver and CNS. Therefore, the use of Compound (I) to increase CoA levels offers the potential to effectively alleviate brain CoA deficiency and restore essential neuronal functions that become compromised in PKAN. Compound (I) is hypothesized to intervene in catabolic processes and prevent the cycle of ineffective catabolism that drives reduced intracellular energy reserves in PA and MMA.

Currently, there are not sufficient approved therapies to treat organic acidemias such as PA or MMA. While diet control and supplementation and transplantation may improve outcomes and quality of life for some patients, diet control is difficult for patients to manage and organ transplantation can put patients at risk of developing debilitating complications. Moreover, despite the documented association of PanK with acyl-CoAs, methods of treating organic acidemias such as PA and MMA using small molecule modulators of CoA levels have yet to be realized. Thus, there remains a need for methods of treating organic acidemias (e.g., PA and/or MMA) via modulation of CoA levels. These needs and others are met by the present disclosure.

For PKAN, there is no approved therapy for the treatment, and no disease-modifying medication is available for clinical use. Current treatment guidelines for PKAN focus on supportive measures for dystonia, including muscle relaxants (anticholinergics, benzodiazepines, and other anti-spasticity agents) and treatments for Parkinson's disease. In more severe cases, surgical ablation procedures (thalamotomy or pallidotomy) largely have been replaced in recent years by surgical deep brain stimulation (DBS), but responses to DBS are mixed. High-dose vitamin B₅ (pantothenate) supplementation is under exploration. Fosmetpantotenate (an investigational phosphor-pantothenate replacement therapy) has not been shown to improve clinical outcomes compared with placebo. Iron chelation with deferiprone to lower brain iron levels also has not provided a significant benefit compared with placebo for clinical endpoints in patients with PKAN. Thus, there is a need for a disease-modifying therapy for PKAN.

### BRIEF SUMMARY

The present disclosure provides methods of treating an organic acidemia (e.g., propionic acidemia (PA) and/or methylmalonic acidemia (MMA)) with a therapeutically effective amount of a compound of formula (I) in a subject. In some embodiments, the subject has propionic acidemia (PA) or methylmalonic acidemia (MMA).

The present disclosure also provides methods of treating Pantothenate Kinase Associated Neurodegeneration (PKAN) with a therapeutically effective amount of a compound of formula (I) in a subject.

Accordingly, in an aspect, the present disclosure provides a method of treating propionic acidemia (PA) and/or methylmalonic acidemia (MMA). The method includes administering to a subject in need thereof a therapeutically effective amount of a compound represented by formula (I):

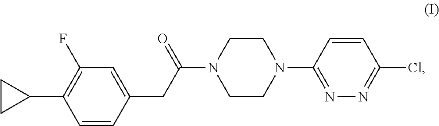 [SMILES: O=C(Cc1ccc(C2CC2)c(F)c1)N1CCN(c2ccc(Cl)nn2)CC1]

a pharmaceutically acceptable salt, hydrate, solvate, or a combination thereof.

In some embodiments, the subject has propionic acidemia (PA) or methylmalonic acidemia (MMA). In some embodiments, the subject is human. In some embodiments, the subject has one more elevated parameters in at least one of plasma, dried blood spot (DBS), and urine, such as an elevated level of 2-methylcitrate or 2-methylcitric acid (MCA), MCA:citrate ratio, methylmalonate or methylmalonic acid (MMA), 3-hydroxypropionate or 3-hydroxypropionic acid (3HP), propionylglycine, tiglylglycine, C3-carnitine, C3:C2-carnitine ratio, ammonium, and/or Fibroblast growth factor 21 (FGF21). In some embodiments, the subject has reduced acetyl-CoA in at least one of plasma, dried blood spot (DBS), and urine. In some embodiments, the subject has one or more mutations in propionyl-CoA carboxylase alpha subunit (PCCA) and/or propionyl-CoA carboxylase beta subunit (PCCB). In some embodiments, the subject has a methylmalonyl-Coenzyme A mutase (MUT) deficiency, such as MUT− (less than a normal amount of MUT enzyme produced) or MUT0 (no MUT enzyme produced). In some embodiments, the subject has a normal level of vitamin B12 in serum or plasma and/or a normal level of homocysteine in plasma.

In another aspect, the present disclosure provides a method of treating Pantothenate Kinase Associated Ncurodcgeneration (PKAN). The method includes administering to a subject in need thereof a therapeutically effective amount of a compound represented by formula (I):

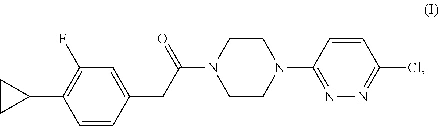 [SMILES: O=C(Cc1ccc(C2CC2)c(F)c1)N1CCN(c2ccc(Cl)nn2)CC1]

a pharmaceutically acceptable salt, hydrate, solvate, or a combination thereof.

In some embodiments, the subject has Pantothenate Kinase Associated Neurodegeneration (PKAN). In some embodiments, the subject has a diagnosis of PKAN as indicated by a confirmed mutation in the pantothenate kinase 2 gene (PANK2). In some embodiments, the subject is human. In some embodiments, the subject is at least 6 years of age. In some embodiments, the subject is at least 18 years of age. In some embodiments, the subject has dystonia. In some embodiments, the subject was diagnosed with dystonia before the age of 10. In some embodiments, the subject has abnormal levels of one or more neural markers such as tau (e.g., in serum). In some embodiments, the subject has elevated tau in serum. In some embodiments, the subject has an elevated pantothenate level in at least one of plasma, serum, and dried blood spot. In some embodiments, the subject has a reduced acetyl-CoA level in at least one of plasma, serum, and dried blood spot.

### BRIEF DESCRIPTION OF THE DRAWINGS

FIG. 1 shows mean (±standard deviation, SD) concentrations of Compound (I) in Pcca^{-/-}PCCA(A138T)^{tg/0} mice plasma (micromolar, μM) and tissues (picomoles per milligram, pmol/mg) after 22.5 parts per million (ppm; 3 milligram per kilogram, mg/kg) of Compound (I) in chow for 30 days after weaning.

FIGS. 2A-2B shows mean (±SD) concentrations of total CoA in Pcca^{-/-}PCCA(A138T)^{tg/0} mice liver (FIG. 2A) and brain (FIG. 2B) (pmol/mg) after 22.5 ppm (3 mg/kg) of Compound (I) in chow for 30 dys after weaning.

FIGS. 3A-3D shows mean (±SD) CoASH (FIG. 3A), C2-CoA (FIG. 3B), C3-CoA (FIG. 3C), and C3:C2 CoA (FIG. 3D) levels in Pcca^{-/-}PCCA(A138T)^{tg/0} mice liver after 22.5 ppm (3 mg/kg) of Compound (I) in chow for 30 days after weaning.

FIGS. 4A-4D shows mean (±SD) CoASH (FIG. 4A), C2-CoA (FIG. 4B), C3-CoA (FIG. 4C), and C3:C2 CoA (FIG. 4D) levels in Pcca^{-/-}PCCA(A138T)⁰ mice brain after 22.5 ppm (3 mg/kg) of Compound (I) in chow for 30 days after weaning.

FIGS. 5A-5D shows mean (±SD) CoASH (FIG. 5A), C2-CoA (FIG. 5B), C3-CoA (FIG. 5C), and C3:C2 CoA (FIG. 5D) levels in Pcca^{-/-}PCCA(A138T)⁰ mice heart after 22.5 ppm (3 mg/kg) of Compound (I) in chow for 30 days after weaning.

FIGS. 6A-6D shows mean (±SD) plasma free carnitine (FIG. 6A), plasma C2-camitine (FIG. 6B), plasma C3-camitine (FIG. 6C), and plasma C3:C2 (FIG. 6D) levels in Pcca^{-/-}PCCA(A138T)^{tg/0} mice plasma after 22.5 ppm (3 mg/kg) of Compound (I) in chow for 30 days after weaning.

FIGS. 7A-7D shows mean (±SD) urine free carnitine (FIG. 7A), urine C2-carnitine (FIG. 7B), urine C3-carnitine (FIG. 7C), and urine C3:C2 (FIG. 7D) levels in Pcca-PCCA(A138T)^{tg/0} mice urine after 22.5 ppm (3 mg/kg) of Compound (I) in chow for 30 days after weaning.

FIG. 8 shows mean (±SD) TCA cycle intermediate levels in Pcca^{-/-}PCCA(A138T)^{tg/0} mice plasma after 22.5 ppm (3 mg/kg) of Compound (I) in chow for 30 days after weaning.

FIG. 9 shows mean (±SD) TCA cycle intermediate levels in Pcca 1-PCCA(A138T)^{g}10 mice urine after 22.5 ppm (3 mg/kg) of Compound (I) in chow for 30 days after weaning.

FIG. 10 shows probability of survival in Pcca^{-/-}PCCA(A138T)^{tg/0} mice after 22.5 ppm (3 mg/kg) of Compound (I) in chow for 30 days after weaning.

FIGS. 11A-11F show results of PCR genotyping of brain and liver from SynCre⁺Pank1^{-/-}Pank2^{-/-} mice. Genomic DNA was isolated from forebrains (FIGS. 11A-11C) and livers (FIGS. 11D-11F) of C57B16/J (WT), SynCre⁺Pank1^{fl/fl}Pank2^{fl/fl} (PK1-2 flox Cre+) and SynCre⁰Pank1^{fl/fl}Pank2^{fl/fl} (PK1-2 flox Cre−) mice at P45. Pank1 alleles (a,d), Pank2 alleles (b,e) and the Cre transgene (c,f) were detected with primers specific for unfloxed wild-type and floxed Pank1 and Pank2 genes, and for the SynCre transgene, together with the sizes of the PCR products, as listed in Table 2.

FIGS. 12A-12B show gene transcript levels of Pank isoforms in brain (FIG. 12A) and liver (FIG. 12B) of SynCre⁺Pank1^{-/-}Pank2^{-/-} mice. The levels of the three Pank mRNAs were quantified by real-time PCR using primers listed in Table 3. RNA was isolated from (a) hindbrain or (b) liver of 3 male and 3 female mice that were tail genotyped as SynCre⁺Pank1^{fl/fl} Pank2^{-/-} mice, and transcript abundance was determined relative to Gapdh transcripts in triplicate for each sample. Means (±SD) are plotted. Gray bars are control SynCre⁰Pank1^{fl/fl}Pank2^{fl/fl} mice and red bars are SynCre⁺Pank1^{-/-}Pank2^{-/-} mice. Primer sets are listed in Table 3.

FIGS. 13A-13B show elevation of CoA in mouse forebrain (FIG. 13A) and hindbrain (FIG. 13B). Mean±standard deviation (SD) are shown.

FIG. 14 shows total forebrain CoA levels in wild-type mice and Pank1,2 neural knockout mice with and without Compound (1). Wild-type (WT) mice and Pank1,2 neural knockout mice (PK1,2 dKO) were maintained on chow with and without Compound (I) (75 ppm) starting at postnatal day 14 and CoA levels were assessed at postnatal day 45. Statistical significance was determined using a two-tailed Student's t test (GraphPad software) and P-values are shown above the bars, N=6-8. Black circles show individual animal data and bars depict means±SEM.

FIGS. 15A-15B show effects of Compound (T) therapy on distance travelled (FIG. 15A) and percent of time moving (FIG. 15B) in an open field test by Pank1,2 neural knockout mice.

FIG. 16 shows the percent change in body weight of Pank1,2 neural knockout mice with and without Compound (I). Pank1,2 neural knockout mice (PK1,2 dKO) were maintained on chow with and without Compound (I) 75 ppm) starting at postnatal day 14. Data are means±SEM and statistical significance was determined using a two-tailed Student's t test (GraphPad software). The P-value is shown on the figure. The black horizontal bar shows zero.

FIG. 17 shows the probability of survival of Pank1,2 neural knockout mice with and without Compound (I). Probability of survival over 45 days is shown in untreated Pank1,2 neural knockout mice (PK1,2 dKO; black line) and mice treated with Compound (I) (blue line). Animals were maintained on chow with and without Compound (1) (75 ppm) starting at postnatal day 14. Compound (I) treated mice did not reach median survival lifespan as the animals were harvested for experimental determination at postnatal day 45.

FIG. 18 shows study design for a single ascending dose (SAD) cohort of the clinical study of Example 4.

FIG. 19 shows study design for a food effect (FE) cohort of the clinical study of Example 4.

FIG. 20 shows study design for a multiple ascending dose (MAD) cohort of the clinical study of Example 4.

FIG. 21 shows study design for a propionic acidemia (PA) and methylmalonic acidemia (MMA) cohort of a clinical study of Example 4.

FIG. 22 shows dose adjustments for the PA/MMA cohort in the event of neutropenia in the clinical study of Example 4.

FIG. 23 shows how expression of PanK activation by Compound (I) is expected to increase acyl-CoAs (products of CoA biosynthesis), including acetyl-CoA, and reduce pantothenate, the substrate for CoA biosynthesis.

FIG. 24 shows the pharmacokinetics upon repeated dosing of Compound (I) as described in Example 4.

FIGS. 25A-25B show the PK-PD relationship of Compound (I) and plasma pantothenate (FIG. 25A) or whole blood acetyl-CoA (FIG. 25B).

### DETAILED DESCRIPTION

### I. General

The present disclosure provides methods of treating an organic acidemia (e.g., propionic acidemia (PA) and/or methylmalonic acidemia (MMA)) with a therapeutically effective amount of a compound of formula (I) in a subject. In some embodiments, the subject has propionic acidemia (PA) or methylmalonic acidemia (MMA). In some embodiments, the subject has one or more elevated biomarkers (e.g., 2-methylcitrate or 2-methylcitric acid (MCA), MCA:citrate ratio, methylmalonate or methylmalonic acid (MMA), 3-hydroxypropionate or 3-hydroxypropionic acid (3HP), propionylglycine, tiglylglycine, C3-carnitine, C3:C2-carnitine ratio, ammonium, pantothenate, tricyclic acid (TCA) cycle intermediates, Fibroblast growth factor 21 (FGF21), and/or B-type natriuretic peptide BNP)). In some embodiments, the subject has a reduced acetyl-CoA level. The compound of formula (I) can be administered to the subject orally in one or multiple doses daily (e.g., once, twice, three times, or four times daily). In some instances, the therapeutically effective amount of the compound of formula (I) can reduce or substantially normalize elevated biomarkers (e.g., MCA, MCA:citrate ratio, MMA, 3HP, propionylglycine, tiglylglycine, C3-camitine, C3:C2-carnitine ratio, ammonium, pantothenate, tricyclic acid (TCA) cycle intermediates, FGF21, and/or BNP) in the subject having PA and/or MMA. In some instances, the therapeutically effective amount of the compound of formula (I) can increase or substantially normalize a reduced acetyl-CoA level in the subject having PA and/or MMA. In some instances, the therapeutically effective amount of the compound of formula (I) can substantially reduce symptoms associated with PA and/or MMA in the subject. In some instances, the therapeutically effective amount of the compound of formula (I) can prevent or obviate the need for a liver, kidney, or combined liver-kidney transplantation in the subject having PA or MMA.

The present disclosure also provides methods of treating Pantothenate Kinase Associated Neurodegeneration (PKAN) with a therapeutically effective amount of a compound of formula (I) in a subject. In some embodiments, the subject's diagnosis of PKAN is indicated by a confirmed mutation in the pantothenate kinase 2 gene (PANK2). In some embodiments, the subject is human. In some embodiments, the subject is at least 6 years of age. In some embodiments, the subject is at least 18 years of age. In some embodiments, the subject has dystonia. In some embodiments, the subject was diagnosed with dystonia before the age of 10. In some embodiments, the subject has abnormal levels of one or more neural markers such as tau (e.g., in serum). In some embodiments, the subject has elevated tau in serum. In some embodiments, the subject has an elevated pantothenate level in at least one of plasma, serum, and dried blood spot. In some embodiments, the subject has a reduced acetyl-CoA level in at least one of plasma, serum, and dried blood spot. In some embodiments, the therapeutically effective amount of the compound of formula (I) can reduce or substantially normalize elevated biomarkers such as elevated pantothenate levels. In some embodiments, the therapeutically effective amount of the compound of formula (I) can increase or substantially normalize depressed biomarkers such as reduced acetyl-CoA levels. In some embodiments, the therapeutically effective amount of the compound of formula (I) can substantially reduce symptoms associated with PKAN in the subject.

### II. Definitions

As used herein, the terms below have the meanings indicated.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 1 mg to 3 mg (milligram),” which is intended to include 1 mg, 3 mg, and everything in between to any number of significant figures (e.g., 1.255 mg, 2.1 mg, 2.9999 mg, etc.).

“Salt” refers to acid or base salts of the compounds of the present disclosure. Illustrative examples of pharmaceutically acceptable acid addition salts are mineral acid (hydrochloric acid, hydrobromic acid, phosphoric acid, and the like) salts and organic acid (acetic acid, propionic acid, glutamic acid, citric acid, and the like) salts. Examples of pharmaceutically acceptable base addition salts include sodium, potassium, calcium, ammonium, organic amino, or magnesium salt, or a similar salt. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

“Solvate” refers to a compound provided herein or a salt thereof, that further includes a stoichiometric or non-stoichiometric amount of solvent bound by non-covalent intermolecular forces.

“Hydrate” refers to a compound that is complexed to a water molecule. The compounds of the present disclosure can be complexed with ½ water molecule or from 1 to 10 water molecules.

“Pharmaceutically acceptable” refers to those compounds (salts, hydrates, solvates, stereoisomers, conformational isomers, tautomers, etc.) which are suitable for use in contact with the tissues of patients without undue toxicity, irritation, and allergic response, are commensurate with a reasonable benefit/risk ratio, and are effective for their intended use. The compounds disclosed herein can exist as pharmaceutically acceptable salts, as defined and described herein.

“PanK allosteric modulator” or “PanK modulator” is used herein to refer to a compound that exhibits modulatory activity (e.g., inhibitory or activating activity) of a PanK enzyme. A PanK modulator having inhibitory activity can have an IC₅₀ with respect to pantothenate kinase (PanK) activity of no more than about 1 micromolar (M) and more typically not more than about 50 nM, as measured in the PanK assay described generally in International Patent Application Nos. PCT/US2017/223474 and PCT/US2018/067539 (e.g., the enzymatic activity of recombinant human PanK of Table 1 in each PCT application). In particular, Compound (I) exhibits an IC₅₀ of less than 5 nM against hPanK3, as disclosed in PCT/US2018/067539. “IC₅₀” is that concentration of inhibitor or a modulator having inhibitory activity (e.g., an allosteric modulator) which reduces the activity of an enzyme (e.g., PanK) to half-maximal level.

“Composition,” as used herein, is intended to encompass a product comprising the specified ingredients in the specified amounts, as well as any product, which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

“Pharmaceutically acceptable excipient” refers to a substance that aids the administration of an active agent to and absorption by a subject. Pharmaceutical excipients useful in the present disclosure include, but are not limited to, binders, fillers, glidants, disintegrants, surfactants, lubricants, coatings, sweeteners, flavors, and colors. One of skill in the art will recognize that other pharmaceutical excipients are useful in the present disclosure.

“Tablet” refers to solid pharmaceutical formulations with and without a coating. The term “tablet” also refers to tablets having one, two, three or even more layers, wherein each of the before mentioned types of tablets may be without or with one or more coatings. In some embodiments, tablets of the present disclosure can be prepared by roller compaction or other suitable means known in the art. The term “tablet” also comprises mini, melt, chewable, effervescent, and orally disintegrating tablets. Tablets include the compound of formula (I) and one or more pharmaceutical excipients (e.g., fillers, binders, glidants, disintegrants, surfactants, binders, lubricants, and the like). Optionally, a coating agent can be also included. For the purposes of calculating percent weight of the tablet formulation, the amount of coating agent is not included in the calculation. That is, the percent weights reported herein are of the uncoated tablet.

“Administering” refers to therapeutic provision of the compound or a form thereof to a subject, such as by oral administration.

“Patient” or “subject” refers to a living organism suffering from or prone to a disease or condition that can be treated by administration of a pharmaceutical composition as provided herein. Non-limiting examples include humans, non-human primates (e.g., monkeys), goats, pigs, sheep, cows, deer, horses, bovines, rats, mice, rabbits, hamsters, guinea pigs, cats, dogs, and other non-mammalian animals. In some embodiments, the subject is human. In some embodiments, a subject is an adult (e.g., at least 18 years of age).

“Therapeutically effective amount” refers to an amount of a compound or of a pharmaceutical composition useful for treating or ameliorating an identified disease or condition, or for exhibiting a detectable therapeutic or inhibitory effect. The exact amounts will depend on the purpose of the treatment, and will be ascertainable by clinicians, pharmacists, and the like (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Treat”, “treating,” and “treatment” refer to any indicia of success in the treatment or amelioration of an injury, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, assay (e.g., analysis of a fluid of a subject, such as blood, plasma, or urine), imaging analysis, neuropsychiatric exams, and/or a psychiatric evaluation.

“About” means a range of values including the specified value, which a person of ordinary skill in the art would consider reasonably similar to the specified value. In some embodiments, the term “about” means within a standard deviation using measurements generally acceptable in the art. In some embodiments, about means a range extending to +/−10% of the specified value. In some embodiments, about means the specified value.

Unless specifically indicated otherwise, the content of the compound of formula (I) in, e.g., a tablet formulation is calculated based on the normalized weight of the compound of formula (I) on a salt-free and anhydrous basis. That is, the salt and/or water content in the compound of formula (I) is not included in the calculation.

“A,” “an,” or “a(n)”, when used in reference to a group of substituents or “substituent group” herein, mean at least one. For example, where a compound is substituted with “an” alkyl or aryl, the compound is substituted with at least one alkyl and/or at least one aryl, wherein each alkyl and/or aryl is optionally different. In another example, where a compound is substituted with “a” substituent group, the compound is substituted with at least one substituent group, wherein each substituent group is optionally different.

### III. Methods of Treating Organic Acidemias

In a first aspect, the present disclosure provides a method of treating an organic acidemia (e.g., propionic acidemia, methylmalonic acidemia, glutaric acidemia, isovaleric acidemia, HMG-CoA lyase deficiency, and/or defects in fatty acid oxidation enzymes). The method includes administering to a subject in need thereof a therapeutically effective amount of a compound represented by formula (I):

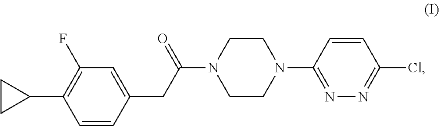 [SMILES: O=C(Cc1ccc(C2CC2)c(F)c1)N1CCN(c2ccc(Cl)nn2)CC1]

or a pharmaceutically acceptable salt, hydrate, solvate, or a combination thereof.

In an aspect, the present disclosure provides a method of treating propionic acidemia (PA) and/or methylmalonic acidemia (MMA). The method includes administering to a subject in need thereof a therapeutically effective amount of the compound represented by formula (I), a pharmaceutically acceptable salt, hydrate, solvate, or a combination thereof.

In some embodiments, the method includes administering to a subject in need thereof a therapeutically effective amount of the compound represented by formula (I), a pharmaceutically acceptable salt thereof.

### III-1: Compound of Formula (I)

The compound of formula (I) can be in a pharmaceutically acceptable salt form or in a neutral form, each of which is optionally in a solvate or a hydrate form.

In some embodiments, the compound of formula (I) is in a pharmaceutically acceptable salt form. In some embodiments, a pharmaceutically acceptable acid addition salt of the compound of formula (I) is represented by formula (Ia):

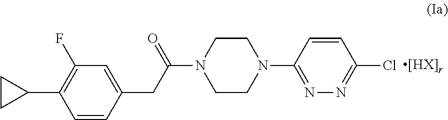 [SMILES: O=C(Cc1ccc(C2CC2)c(F)c1)N1CCN(c2ccc(Cl)nn2)CC1]

wherein HX is a pharmaceutically acceptable acid addition and subscript n is 1, 2, or 3. In some embodiments, subscript n is 1 and the compound of formula (I) is in a mono salt form.

Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like.

In some embodiments, the compound of formula (I) is in a neutral form.

In some embodiments, the compound of formula (I) is represented by the formula:

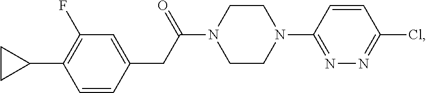 [SMILES: O=C(Cc1ccc(C2CC2)c(F)c1)N1CCN(c2ccc(Cl)nn2)CC1]

having the name of 1-(4-(6-chloropyridazin-3-yl)piperazin-1-yl)-2-(4-cyclopropyl-3-fluorophenyl)ethan-1-one.

In some embodiments, the compound of formula (I) has a purity of at least about 95 area % determined by a chiral high-performance liquid chromatography (HPLC). In some embodiments, the compound of formula (I) has a purity of from about 95 area % to about 100 area %, from about 96 area % to about 100 area %, from about 97 area % to about 100 area %, from about 98 area % to about 100 area %, or from about 99 area % to about 100 area %, determined by a chiral high-performance liquid chromatography (HPLC). In some embodiments, the compound of formula (I) has a purity of from about 99 area % to about 100 area %. In some embodiments, the compound of formula (I) has a purity of about 99.0 area %, about 99.1 area %, about 99.2 area %, about 99.3 area %, about 99.4 area %, about 99.5 area %, about 99.6 area %, about 99.7 area %, about 99.8 area %, about 99.9 area %, to about 100.0 area %.

In some embodiments, the compound of formulae (I) or (Ta) is in a solvate and/or a hydrate form.

### III-2: Subject

The subject can have propionic acidemia (PA) and/or methylmalonic acidemia (MMA).

In some embodiments, the subject has propionic acidemia (PA). In some embodiments, the subject has methylmalonic acidemia (MMA).

The subject can have one or more elevated biomarkers (e.g., MCA, MCA:citrate ratio, MMA, 3HP, propionylglycine, tiglylglycine, C3-camitine, C3:C2-carnitine ratio, ammonium, pantothenate, tricyclic acid (TCA) cycle intermediates, FGF21, and/or BNP). The subject can have a reduced acetyl-CoA level.

In some embodiments, the subject has an elevated level of 2-methylcitrate or 2-methylcitric acid (MCA), MCA:citrate ratio, methylmalonate or methylmalonic acid (MMA), 3-hydroxypropionate or 3-hydroxypropionic acid (3HP), propionylglycine, tiglylglycine, C3-camitine, C3:C2-carnitine ratio, ammonium, and/or Fibroblast growth factor 21 (FGF21), each of which is detected at least in one of plasma, dried blood spot (DBS), and urine.

In some embodiments, the subject has an elevated 2-methylcitrate or 2-methylcitric acid (MCA) and/or an elevated ratio of MCA:citrate at least in one of plasma, dried blood spot (DBS), and urine. In some embodiments, the subject has an elevated 2-methylcitrate or 2-methylcitric acid (MCA) and/or an elevated ratio of MCA:citrate plasma. In some embodiments, the subject has an elevated MCA at least in one of plasma, dried blood spot (DBS), and urine. In some embodiments, the subject has an elevated MCA in plasma. In some embodiments, the subject has an elevated MCA:citrate ratio at least in one of plasma, dried blood spot (DBS), and urine. In some embodiments, the subject has an elevated MCA:citrate ratio in plasma. In some embodiments, the subject has an elevated MCA and MCA:citrate ratio at least in one of plasma, dried blood spot (DBS), and urine. In some embodiments, the subject has an elevated MCA and MCA:citrate ratio in plasma.

In some embodiments, the subject has an elevated methylmalonate or methylmalonic acid (MMA) at least in one of plasma, dried blood spot (DBS), and urine. In some embodiments, the subject has an elevated MMA in plasma.

In some embodiments, the subject has an elevated 3-hydroxypropionate or 3-hydroxypropionic acid (3HP) at least in one of plasma, dried blood spot (DBS), and urine. In some embodiments, the subject has an elevated 3HP in plasma.

In some embodiments, the subject has an elevated propionylglycine and/or tiglylglycine at least in one of plasma, dried blood spot (DBS), and urine. In some embodiments, the subject has an elevated propionylglycine at least in one of plasma, dried blood spot (DBS), and urine. In some embodiments, the subject has an elevated tiglylglycine at least in one of plasma, dried blood spot (DBS), and urine. In some embodiments, the subject has an elevated both propionylglycine and tiglylglycine at least in one of plasma, dried blood spot (DBS), and urine.

In some embodiments, the subject has an elevated propionylcarnitine (C3-carnitine) level, a reduced acetylcarnitine (C2-carnitine) level, a reduced carnitine level, and/or an elevated C3:C2-carnitine ratio at least in one of plasma, serum, and dried blood spot (DBS). In some embodiments, the subject has an elevated C3-carnitine level, a reduced C2-carnitine level, a reduced camitine level, and/or an elevated C3:C2-carnitine ratio in plasma. In some embodiments, the subject has an elevated C3-carnitine level, a reduced C2-carnitine level, a reduced camitine level, and/or an elevated C3:C2-carnitine ratio in the liver. In some embodiments, the subject has an elevated C3:C2-Coenzyme A (CoA) level in the liver. In some embodiments, the subject has an elevated C3-CoA level in the liver and/or heart. In some embodiments, the subject has an elevated C3-carnitine, a reduced C2-carnitine, and/or an elevated C3:C2-camitine ratio at least in one of plasma, serum, and dried blood spot (DBS). In some embodiments, the subject has an elevated C3-carnitine, and/or an elevated C3:C2-carnitine ratio at least in one of plasma, serum, and dried blood spot (DBS). In some embodiments, the subject has an elevated C3-carnitine at least in one of plasma, serum, and dried blood spot (DBS). In some embodiments, the subject has an elevated C3-carnitine in plasma. In some embodiments, the subject has an elevated C3:C2-carnitine ratio at least in one of plasma, serum, and dried blood spot (DBS). In some embodiments, the subject has an elevated C3:C2-carnitine ratio in plasma. In some embodiments, the subject has an elevated C3-carnitine and C3:C2-camitine ratio at least in one of plasma, serum, and dried blood spot (DBS). In some embodiments, the subject has an elevated C3-carnitine and C3:C2-carnitine ratio in plasma.

In some embodiments, the subject has an elevated ammonium at least in one of plasma, serum, and dried blood spot (DBS). In some embodiments, the subject has an elevated ammonium in plasma.

In some embodiments, the subject has an elevated Fibroblast growth factor 21 (FGF21) at least in one of plasma, serum, and dried blood spot (DBS). In some embodiments, the subject has an elevated FGF21 in plasma.

In some embodiments, the subject has an elevated pantothenate. In some embodiments, the subject has an elevated pantothenate in plasma.

In some embodiments, the subject has a reduced acetyl-CoA level. In some embodiments, the subject has a reduced acetyl-CoA level in whole blood.

The subject can have other elevated biomarkers, such as an elevated level of tricyclic acid (TCA) cycle intermediates (e.g., citrate, ketoglutarate, succinate, malate, etc.) and/or B-type natriuretic peptide (BNP). The subject can also have abnormal levels of lactate, blood gas, and/or amino acids.

In some embodiments, the subject has one or more TCA cycle metabolites elevated. In some embodiments, the subject has one or more TCA cycle metabolites reduced. In some embodiments, the one or more TCA cycle metabolites are selected from the group consisting of α-ketoglutarate, citrate, fumarate, isocitrate, malate, methylcitrate, methylmalonate, oxaloacetate, succinate, glucose, glycerate, phenylpyruvate, phosphoenolpyruvate (PEP), lactate, glucosamine, choline, creatinine, and creatine. In some embodiments, the one or more TCA cycle metabolites are selected from the group consisting of α-ketoglutarate, citrate, isocitrate, malate, methylcitrate, methylmalonate, oxaloacetate, succinate, glucose, glycerate, phosphoenolpyruvate (PEP), and choline. In some embodiments, the one or more TCA cycle metabolites are selected from the group consisting of α-ketoglutarate, malate, methylcitrate, methylmalonate, oxaloacetate, and succinate. In some embodiments, the one or more TCA cycle metabolites are selected from the group consisting of α-ketoglutarate, citrate, succinate, and malate. In some embodiments, the one or more TCA cycle metabolites include malate. In some embodiments, the malate level is elevated. In some embodiments, the levels of TCA cycle metabolites of the one or more TCA cycle metabolites are urinary levels. In some embodiments, the levels of TCA cycle metabolites of the one or more TCA cycle metabolites are plasma levels.

In some embodiments, the subject has elevated malate, methylcitrate, citrate, α-ketoglutarate, succinate, glycine, and/or methylmalonate levels in urine and/or plasma. In some embodiments, the subject has elevated malate, methylcitrate, citrate, α-ketoglutarate, succinate, glycine, and/or methylmalonate levels in urine. In some embodiments, the subject has elevated malate, methylcitrate, citrate, α-ketoglutarate, succinate, glycine, and/or methylmalonate levels in plasma. In some embodiments, the subject has elevated malate, methylcitrate, citrate, α-ketoglutarate, succinate, glycine, and/or methylmalonate levels in urine and plasma. In some embodiments, the subject has elevated malate, citrate, α-ketoglutarate, and/or succinate in urine and/or plasma. In some embodiments, the subject has elevated malate, citrate, α-ketoglutarate, and/or succinate in urine. In some embodiments, the subject has elevated malate, citrate, α-ketoglutarate, and/or succinate in plasma. In some embodiments, the subject has elevated malate, citrate, α-ketoglutarate, and/or succinate in urine and plasma.

In some embodiments, the subject has an elevated B-type natriuretic peptide (BNP). In some embodiments, the subject has an elevated BNP in plasma.

In some embodiments, the subject has abnormal levels of lactate, blood gas, and/or amino acids. In some embodiments, the subject has abnormal levels of lactate, blood gas, and/or amino acids in plasma.

The subject can have one or more mutations in propionyl-CoA carboxylase alpha subunit (PCCA) and/or propionyl-CoA carboxylase beta subunit (PCCB), which are confirmed by molecular genetic testing. In some embodiments, the subject has one or more mutations in propionyl-CoA carboxylase alpha subunit (PCCA) and/or propionyl-CoA carboxylase beta subunit (PCCB), which are optionally confirmed by molecular genetic testing.

In some embodiments, the subject has a methylmalonyl-Coenzyme A mutase (MUT) deficiency, which is optionally confirmed by molecular genetic testing. In some embodiments, the subject having a MUT deficiency has MMA. In some embodiments, the MUT deficiency is caused by one or more changes or mutations in a MUT gene, which can cause no MUT enzyme (MUT0) or less than a normal amount of the MUT enzyme (MUT−) to be produced.

In some embodiments, the subject has a normal level of vitamin B12 in serum or plasma and/or a normal level of homocysteine in plasma.

In some embodiments, the subject has undergone a liver transplant. In some embodiments, the subject has undergone a kidney transplant. In some embodiments, the subject has undergone a liver transplant and a kidney transplant. In some embodiments, the subject who has undergone a kidney and/or liver transplant has MMA. In some embodiments, the subject who has undergone a kidney and/or liver transplant has PA.

In some embodiments, the subject has not undergone a liver transplant. In some embodiments, the subject has not undergone a kidney transplant. In some embodiments, the subject has not undergone a combined liver and kidney transplant.

In some embodiments, the subject having propionic acidemia (PA) and/or methylmalonic acidemia (MMA) meets the following criteria as described below.

In some embodiments, the subject has confirmed PA diagnosed by all of the following criteria (may be based on historical records):
a. Elevated plasma/DBS/urine 2-methylcitrate and/or 3-hydroxypropionate;
b. Elevated plasma/serum/DBS propionyl carnitine; and
c. PCCA/PCCB mutations confirmed by molecular genetic testing.

In some embodiments, the subject having MMA has elevated plasma MMA levels.

In some embodiments, the subject has confirmed MMA diagnosed by all of the following criteria (may be based on historical records):
a. MUT deficiency (MUT0 or MUT−);
b. Elevated plasma, serum, DBS, or urine MMA levels;
c. Presence of normal serum or plasma vitamin B12 and plasma homocysteine levels; and
d. Confirmed by molecular genetic testing.

In some embodiments, the subject does not require concurrent use of a strong or moderate CYP3A4 inhibitor. In some embodiments, the subject does not require concurrent use of a strong CYP2C19 inhibitor.

In some embodiments, the subject having propionic acidemia (PA) and/or methylmalonic acidemia (MMA) does not have one or more conditions selected from the group consisting of:
a) alanine aminotransferase (ALT) and/or aspartate aminotransferase (AST) exceed two times of a upper limit of normal (ULN);
b) total bilirubin exceeds two times of a upper limit of normal (ULN);
c) an international normalized ratio (INR) is more than 1.4;
d) a baseline estimated glomerular filtration rate (eGFR) is less than 45 mL/min calculated using the CKD-EPI formula;
e) a positive test result for hepatitis B surface antigen, hepatitis C virus antibody, or HIV types 1 or 2 antibodies; and
f) a positive test result for SARS-CoV-2.

Further inclusion and exclusion criteria for subjects who may benefit from treatment with a compound of formula (I), such as subjects enrolled in a First-in-human, Randomized, Placebo-Controlled, and/or Single and Multiple Ascending Dose Study, are described in Example 4.

In some embodiments, the subject meets all of inclusion criteria of 1) to 9) and 12) to 16) as described in Example 4. In some embodiments, the subject meets all of inclusion criteria of 1) to 9) and 12) to 16) as described in Example 4, provided that the subject does not meet any one of exclusion criteria of 1) to 23) as described in Example 4.

In some embodiments, the subject is a mammal. In some embodiments, the subject is human.

### III-3: Treatment Cycle and Dose Adjustment

Treatment with the compound of formula (I) can include one or more treatment cycles (e.g., 1 to 6 treatments, such as at least 1, 2, 3, 4, 5, 6, or more treatment cycles). In some embodiments, the treatment includes one or more treatment cycles (e.g., 1 to 6 treatments, such as at least 1, 2, 3, 4, 5, 6, or more treatment cycles). In some embodiments, the treatment includes at least 2, 3, 4, 5, 6, or more treatment cycles. In some embodiments, the treatment includes 2 to 6 treatment cycles. In some embodiments, the treatment includes 3 to 6 treatment cycles. In some embodiments, the treatment includes 3 to 4 treatment cycles. In some embodiments, the treatment includes 4 to 6 treatment cycles. In some embodiments, the treatment includes 5 to 6 treatment cycles. In some embodiments, the treatment includes 3 treatment cycles. In some embodiments, the treatment includes 4 treatment cycles. In some embodiments, the treatment includes 4 treatment cycles. In some embodiments, the treatment includes 6 treatment cycles. In some embodiments, the treatment is chronic (e.g., the compound of formula (I) is administered to the subject on a regular basis according to a prescribed treatment schedule, as described herein).

After a previous treatment cycle or a first period of treatment with the compound of formula (I), a dose of the compound of formula (I) can be adjusted (e.g., dose escalation or de-escalation) or remain unchanged (e.g., no dose adjustment). Dose adjustment or lack thereof may be based at least in part on a safety evaluation (e.g., a dose-limiting toxicity (DLT) assessment) and/or an absolute neutrophil count (ANC). In some embodiments, dose adjustment comprises dose escalation. In some embodiments, dose adjustment comprises dose de-escalation.

In some embodiments, the administration of the compound of formula (I) includes a dose escalation or de-escalation after a previous treatment cycle, wherein the dose escalation or de-escalation is determined by a dose-limiting toxicity (DLT) assessment. In some embodiments, the administration of the compound of formula (I) includes a dose escalation after a previous treatment cycle, if safety assessment meets accepted criteria of Example 4. In some embodiments, the administration of the compound of formula (I) includes a dose escalation in a second treatment after a first treatment cycle if safety assessment meets accepted criteria of Example 4. In some embodiments, the administration of the compound of formula (I) includes a dose escalation in a third treatment after a second treatment cycle if safety assessment meets accepted criteria of Example 4. In some embodiments, the administration of the compound of formula (I) includes a dose escalation in a fourth treatment after a third treatment cycle if safety assessment meets accepted criteria of Example 4. In some embodiments, the administration of the compound of formula (I) includes a dose escalation in a fifth treatment after a fourth treatment cycle if safety assessment meets accepted criteria of Example 4. In some embodiments, the administration of the compound of formula (I) includes a dose escalation in a sixth treatment after a fifth treatment cycle if safety assessment meets accepted criteria of Example 4. In some embodiments, the administration of the compound of formula (I) includes a dose de-escalation in a third treatment after a second treatment cycle according to the criteria of Example 4. In some embodiments, the administration of the compound of formula (I) includes a dose de-escalation in a fourth treatment after a third treatment cycle according to the criteria of Example 4. In some embodiments, the administration of the compound of formula (I) includes a dose de-escalation in a fifth treatment after a fourth treatment cycle according to the criteria of Example 4. In some embodiments, the administration of the compound of formula (I) includes a dose de-escalation in a sixth treatment after a fifth treatment cycle according to the criteria of Example 4. In some embodiments, the administration of the compound of formula (I) includes a dose de-escalation within a treatment cycle, according to the criteria of Example 4.

In some embodiments, the administration of the compound of formula (I) includes a dose escalation, a dose de-escalation, or no dose adjustment after a previous treatment cycle, wherein the dose escalation, dose de-escalation, or no dose adjustment is determined by an absolute neutrophil count (ANC).

In some embodiments, a) when the ANC is less than 1.5×10⁹/liter (L), but at or more than 1.0×10⁹/L, the administration of the compound of formula (I) is continued at the same dose (e.g., the same dose of the previous treatment cycle). In some embodiments, the administration of the compound of formula (I) is continued at the same dose while monitoring the ANC (e.g., until it reaches at or exceeds 1.5×10⁹/L). In some embodiments, in a), dose escalation of the compound of formula (I) does not take place during the next treatment cycle, such that the administration of the compound of formula (I) continues at the same dose of the previous treatment cycle in the subsequent treatment cycle. In some embodiments, b) when the ANC is less than 1.0×10⁹/L, but at or more than 0.5×10⁹/L, the administration of the compound of formula (I) is at least temporarily discontinued. In some embodiments, the administration of the compound of formula (I) is discontinued until the ANC reaches or exceeds 1.5×10⁹/L. In some embodiments, in b), when the ANC has increased to at least 1.5×10⁹/L within about 14 days after discontinuation, the administration of the compound of formula (I) is resumed, optionally at a lower dose, further optionally provided that the previous treatment is a first treatment cycle, then the same dose is administered. In some embodiments, in b), when the ANC has not increased to at least 1.5×10⁹/L within about 14 days after discontinuation, the administration of the compound of formula (I) remains at least temporarily discontinued (e.g., until the neutropenia event is resolved). In some embodiments, c) when the ANC is less than 0.5×10⁹/L (e.g., as measured with a first measurement on a first day of the treatment cycle and confirmed by a second measurement on a second day of the treatment cycle), the administration of the compound of formula (I) is at least temporarily discontinued. In some embodiments, in c), when the ANC has increased to at least 1.5×10⁹/L within about 3 days after discontinuation, the administration of the compound of formula (I) is resumed, optionally at a lower dose, further optionally provided that the previous treatment is a first treatment cycle, then the same dose is administered. In some embodiments, in c), when the ANC has not increased to at least 1.5×10⁹/L within about 3 days after discontinuation, the administration of the compound of formula (I) remains at least temporarily discontinued (e.g., until the neutropenia event is resolved). In some embodiments, in c), when the ANC has not increased to at least 1.5×10⁹/L within about 3 days after discontinuation, the administration of the compound of formula (I) is permanently discontinued.

In some embodiments, the administration of the compound of formula (I) includes 1 to 6 dose escalations, optionally 1 to 2 dose de-escalations. In some embodiments, the administration of the compound of formula (I) includes 1 to 6 dose escalations. In some embodiments, the administration of the compound of formula (I) includes 1 to 5 dose escalations. In some embodiments, the administration of the compound of formula (I) includes 2 to 5 dose escalations. In some embodiments, the administration of the compound of formula (I) includes 3 to 5 dose escalations. In some embodiments, the administration of the compound of formula (I) includes 4 to 5 dose escalations. In some embodiments, the administration of the compound of formula (I) includes 5 dose escalations. In some embodiments, the administration of the compound of formula (I) includes 1 to 2 dose de-escalations. In some embodiments, the administration of the compound of formula (I) includes one (1) dose de-escalations.

In some embodiments, each of one or more treatment cycles has a duration of from about 7 to about 14 days and the compound of formula (I) is administered daily. In some embodiments, the first treatment cycle has a duration of from about 7 to about 14 days and the compound of formula (I) is administered daily. In some embodiments, the second treatment cycle has a duration of from about 7 to about 14 days and the compound of formula (I) is administered daily. In some embodiments, the third treatment cycle has a duration of from about 7 to about 14 days and the compound of formula (I) is administered daily. In some embodiments, the fourth treatment cycle has a duration of from about 7 to about 14 days and the compound of formula (I) is administered daily. In some embodiments, the fifth treatment cycle has a duration of from about 7 to about 14 days and the compound of formula (I) is administered daily. In some embodiments, the sixth treatment cycle has a duration of from about 7 to about 14 days and the compound of formula (I) or is administered daily.

In some embodiments, each of one or more treatment cycles has a duration of about 14 days and the compound of formula (I) is administered daily (e.g., once, twice, three times, or four times daily). In some embodiments, the first treatment cycle has a duration of about 7 days and the compound of formula (I) is administered daily (e.g., once, twice, three times, or four times daily). In some embodiments, the second treatment cycle has a duration of about 7 days and the compound of formula (I) is administered daily (e.g., once, twice, three times, or four times daily). In some embodiments, the third treatment cycle has a duration of about 7 days and the compound of formula (I) is administered daily (e.g., once, twice, three times, or four times daily). In some embodiments, the fourth treatment cycle has a duration of about 7 days and the compound of formula (I) is administered daily (e.g., once, twice, three times, or four times daily). In some embodiments, the fifth treatment cycle has a duration of about 14 days and the compound of formula (I) is administered daily (e.g., once, twice, three times, or four times daily). In some embodiments, the sixth treatment cycle has a duration of about 7 days and the compound of formula (I) or is administered daily (e.g., once, twice, three times, or four times daily).

### III-4: Therapeutically Effective Amount/Administration

In some embodiments, the therapeutically effective amount is a total daily dosage of no more than about 2000 mg of the compound of formula (I) on a salt-free and anhydrous basis. In some embodiments, the therapeutically effective amount is a total daily dosage of from about 1 mg to about 2000 mg, from about 5 mg to about 2000 mg, from about 10 mg to about 2000 mg, from about 20 mg to about 2000 mg, from about 30 mg to about 2000 mg, from about 40 mg to about 2000 mg, from about 50 mg to about 2000 mg, from about 1 mg to about 1000 mg, from about 5 mg to about 1000 mg, from about 10 mg to about 1000 mg, from about 20 mg to about 1000 mg, from about 30 mg to about 1000 mg, from about 40 mg to about 1000 mg, from about 50 mg to about 1000 mg, from about 1 mg to about 500 mg, from about 2 mg to about 500 mg, from about 3 mg to about 500 mg, from about 5 mg to about 500 mg, from about 10 mg to about 500 mg, from about 20 mg to about 500 mg, from about 10 mg to about 300 mg, from about 10 mg to about 200 mg, from about 10 mg to about 120 mg, from about 20 mg to about 120 mg, from about 30 mg to about 120 mg, from about 40 mg to about 120 mg, from about 50 mg to about 120 mg, from about 60 mg to about 120 mg, from about 10 mg to about 100 mg, from about 20 mg to about 100 mg, from about 30 mg to about 100 mg, from about 40 mg to about 100 mg, from about 50 mg to about 100 mg, or from about 60 mg to about 100 mg of the compound of formula (I), on a salt-free and anhydrous basis, or any useful range therein. In some embodiments, the therapeutically effective amount is a total daily dosage of from about 1 mg to about 500 mg, from about 2 mg to about 500 mg, from about 3 mg to about 500 mg, from about 5 mg to about 500 mg, from about 10 mg to about 500 mg, from about 20 mg to about 500 mg, from about 10 mg to about 300 mg, from about 10 mg to about 200 mg, from about 10 mg to about 120 mg, from about 20 mg to about 120 mg, from about 30 mg to about 120 mg, from about 40 mg to about 120 mg, from about 50 mg to about 120 mg, from about 60 mg to about 120 mg, from about 10 mg to about 100 mg, from about 20 mg to about 100 mg, from about 30 mg to about 100 mg, from about 40 mg to about 100 mg, from about 50 mg to about 100 mg, or from about 60 mg to about 100 mg of the compound of formula (I), on a salt-free and anhydrous basis, or any useful range therein. In some embodiments, the therapeutically effective amount is a total daily dosage of from about 10 mg to about 200 mg, from about 10 mg to about 120 mg, from about 20 mg to about 120 mg, from about 30 mg to about 120 mg, from about 40 mg to about 120 mg, from about 50 mg to about 120 mg, or from about 60 mg to about 120 mg of the compound of formula (I), on a salt-free and anhydrous basis, or any useful range therein.

In some embodiments, the therapeutically effective amount is a total daily dosage of from about 1 mg to about 500 mg, from about 2 mg to about 500 mg, from about 3 mg to about 500 mg, from about 5 mg to about 500 mg, from about 10 mg to about 500 mg, from about 20 mg to about 500 mg, from about 30 mg to about 500 mg, from about 40 mg to about 500 mg, from about 50 mg to about 500 mg, from about 60 mg to about 500 mg, from about 70 mg to about 500 mg, from about 80 mg to about 500 mg, from about 90 mg to about 500 mg, from about 100 mg to about 500 mg, from about 10 mg to about 300 mg, from about 20 mg to about 300 mg, from about 30 mg to about 300 mg, from about 40 mg to about 300 mg, from about 50 mg to about 300 mg, from about 60 mg to about 300 mg, from about 70 mg to about 300 mg, from about 80 mg to about 300 mg, from about 90 mg to about 300 mg, from about 100 mg to about 300 mg, from about 10 mg to about 200 mg, from about 20 mg to about 200 mg, from about 30 mg to about 200 mg, from about 40 mg to about 200 mg, from about 50 mg to about 200 mg, from about 60 mg to about 200 mg, from about 70 mg to about 200 mg, from about 80 mg to about 200 mg, from about 90 mg to about 200 mg, from about 100 mg to about 200 mg, from about 10 mg to about 120 mg, from about 20 mg to about 120 mg, from about 30 mg to about 120 mg, from about 40 mg to about 120 mg, from about 50 mg to about 120 mg, from about 60 mg to about 120 mg, from about 70 mg to about 120 mg, from about 80 mg to about 120 mg, from about 90 mg to about 120 mg, from about 100 mg to about 120 mg, from about 10 mg to about 100 mg, from about 20 mg to about 100 mg, from about 30 mg to about 100 mg, from about 40 mg to about 100 mg, from about 50 mg to about 100 mg, from about 60 mg to about 100 mg, from about 70 mg to about 100 mg, from about 80 mg to about 100 mg, or from about 90 mg to about 100 mg of the compound of formula (I), on a salt-free and anhydrous basis, or any useful range therein. In some embodiments, the therapeutically effective amount is a total daily dosage of from about 10 mg to about 120 mg, from about 20 mg to about 120 mg, from about 30 mg to about 120 mg, from about 40 mg to about 120 mg, from about 50 mg to about 120 mg, from about 60 mg to about 120 mg, from about 70 mg to about 120 mg, from about 80 mg to about 120 mg, from about 90 mg to about 120 mg, from about 100 mg to about 120 mg, from about 10 mg to about 100 mg, from about 20 mg to about 100 mg, from about 30 mg to about 100 mg, from about 40 mg to about 100 mg, from about 50 mg to about 100 mg, from about 60 mg to about 100 mg, from about 70 mg to about 100 mg, from about 80 mg to about 100 mg, or from about 90 mg to about 100 mg of the compound of formula (I), on a salt-free and anhydrous basis, or any useful range therein. In some embodiments, the therapeutically effective amount is a total daily dosage of from about 50 mg to about 120 mg, from about 60 mg to about 120 mg, from about 70 mg to about 120 mg, from about 80 mg to about 120 mg, from about 90 mg to about 120 mg, from about 100 mg to about 120 mg, from about 50 mg to about 100 mg, from about 60 mg to about 100 mg, from about 70 mg to about 100 mg, from about 80 mg to about 100 mg, or from about 90 mg to about 100 mg of the compound of formula (I), on a salt-free and anhydrous basis, or any useful range therein.

In some embodiments, the therapeutically effective amount is a total daily dosage of about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210, mg, about 220 mg, about 230 mg, about 240 mg, or about 250 mg of the compound of formula (I), on a salt-free and anhydrous basis. In some embodiments, the therapeutically effective amount is a total daily dosage of about 10 mg, about 20 mg, about 30 mg, about 40 mg, about 50 mg, about 60 mg, about 70 mg, about 80 mg, about 90 mg, about 100 mg, about 110 mg, or about 120 mg of the compound of formula (I), on a salt-free and anhydrous basis.

In some embodiments, the therapeutically effective amount is a total daily dosage of about 10 mg of the compound of formula (I), on a salt-free and anhydrous basis. In some embodiments, the therapeutically effective amount is a total daily dosage of about 20 mg of the compound of formula (I), on a salt-free and anhydrous basis. In some embodiments, the therapeutically effective amount is a total daily dosage of about 30 mg of the compound of formula (I), on a salt-free and anhydrous basis. In some embodiments, the therapeutically effective amount is a total daily dosage of about 40 mg of the compound of formula (I), on a salt-free and anhydrous basis. In some embodiments, the therapeutically effective amount is a total daily dosage of about 50 mg of the compound of formula (I), on a salt-free and anhydrous basis. In some embodiments, the therapeutically effective amount is a total daily dosage of about 60 mg of the compound of formula (I), on a salt-free and anhydrous basis. In some embodiments, the therapeutically effective amount is a total daily dosage of about 70 mg of the compound of formula (I), on a salt-free and anhydrous basis. In some embodiments, the therapeutically effective amount is a total daily dosage of about 80 mg of the compound of formula (I), on a salt-free and anhydrous basis. In some embodiments, the therapeutically effective amount is a total daily dosage of about 90 mg of the compound of formula (I), on a salt-free and anhydrous basis. In some embodiments, the therapeutically effective amount is a total daily dosage of about 100 mg of the compound of formula (I), on a salt-free and anhydrous basis. In some embodiments, the therapeutically effective amount is a total daily dosage of about 110 mg of the compound of formula (I), on a salt-free and anhydrous basis. In some embodiments, the therapeutically effective amount is a total daily dosage of about 120 mg of the compound of formula (I), on a salt-free and anhydrous basis.

In some embodiments, the therapeutically effective amount varies based on the age and/or weight of the subject. In some embodiments, subjects who are aged ≥16 years and weigh ≥55 kg are treated with a total daily dosage of about 10 mg, about 20 mg, about 30 mg, about 40 mg, about 50 mg, about 60 mg, about 70 mg, about 80 mg, about 90 mg, about 100 mg, about 110 mg, or about 120 mg of the compound of formula (I), on a salt-free and anhydrous basis. In some embodiments, subjects who weigh ≥35 kg but less than 55 kg will have a lower total daily dosage than subjects who weigh ≥55 kg. In some embodiments, subjects who weigh ≥18 kg but less than 55 kg will have a lower total daily dosage than subjects who weigh ≥55 kg. In some embodiments, subjects who weigh ≥18 kg but less than 35 kg will have a lower total daily dosage than subjects who weigh ≥35 kg. In some embodiments, subjects who are aged ≥12 years but less than 16 years will have a lower total daily dosage than subjects who are aged ≥16 years. In some embodiments, subjects who are aged ≥6 years but less than 12 years will have a lower total daily dosage than subjects who are aged ≥12 years. In some embodiments, subjects who are aged ≥6 years but less than 16 years will have a lower total daily dosage than subjects who are aged ≥16 years.

In general, the compound of formula (I) can be administered orally. In some embodiments, the compound of formula (I) is administered orally. In some embodiments, the compound of formula (I) in a tablet formulation is administered orally.

In general, the compound of formula (I) can be administered once or multiple times (e.g., 2, 3, 4, or more times) daily. In some embodiments, the compound of formula (I) is administered once, twice, three times, or four times daily. In some embodiments, the compound of formula (I) is administered once daily. In some embodiments, the compound of formula (I) is administered twice daily. In some embodiments, the compound of formula (I) is administered three times daily. In some embodiments, the compound of formula (I) is administered four times daily.

The compound of formula (I) can be in an oral dosage form in one or more dosage strengths, where the compound of formula (I) is present in an amount of at least about 1 mg, 5 mg, 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 240 mg, 250 mg, 300 mg, 350 mg, 360 mg, 400 mg, 450 mg, 480 mg, or 500 mg, on a salt-free and anhydrous basis. In some embodiments, the oral dosage form is a tablet formulation in one or more dosage strengths. In some embodiments of the tablet formulation, the compound of formula (I) is present in an amount of from 1 to 1000 mg, from 1 to 750 mg, from 1 to 500 mg, from 1 to 250 mg, from 30 to 1000 mg, from 30 to 750 mg, from 30 to 500 mg, from 30 to 200 mg, from 30 to 180 mg, from 30 to 120 mg, from 30 to 90 mg, from 50 to 1000 mg, from 50 to 750 mg, from 50 to 500 mg, from 50 to 250 mg, from 100 to 1000 mg, from 100 to 750 mg, from 100 to 500 mg, from 100 to 250 mg, from 200 to 1000 mg, from 200 to 750 mg, from 200 to 500 mg, from 300 to 1000 mg, from 300 to 750 mg, from 300 to 500 mg, from 400 to 1000 mg, from 400 to 750 mg, from 500 to 1000 mg, from 500 to 750 mg, from 600 to 1000 mg, from 5 to 250 mg, or from 5 to 100 mg in each tablet, on a salt-free and anhydrous basis. In some embodiments of the tablet formulation, the compound of formula (I) is present in an amount of at least about 1 mg, 5 mg, 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 240 mg, 250 mg, 300 mg, 350 mg, 360 mg, 400 mg, 450 mg, 480 mg, 500 mg, 600 mg, 700 mg, 800 mg, 900 mg, or 1000 mg in each tablet, on a salt-free and anhydrous basis. In some embodiments of the tablet formulation, the compound of formula (I) is present in an amount of about 3 mg, about 5 mg, 10 mg, 20 mg, 30 mg, 50 mg, 100 mg, 120 mg, 150 mg, 200 mg, 240 mg, 250 mg, 300 mg, 400 mg, 500 mg, 600 mg, 700 mg, 800 mg, 900 mg, or 1000 mg in each tablet, on a salt-free and anhydrous basis. In some embodiments of the tablet formulation, the compound of formula (I) is present in an amount of about 10 mg, about 20 mg, about 30 mg, 50 mg, or 100 mg in each tablet, on a salt-free and anhydrous basis.

In some embodiments, the compound of formula (I) is administered once daily to provide a total daily dosage of no more than about 2000 mg of the compound of formula (I). In some embodiments, the compound of formula (I) is administered once daily to provide a total daily dosage of from about 10 mg to about 120 mg, from about 20 mg to about 120 mg, from about 30 mg to about 120 mg, from about 40 mg to about 120 mg, from about 50 mg to about 120 mg, from about 60 mg to about 120 mg, from about 70 mg to about 120 mg, from about 80 mg to about 120 mg, from about 90 mg to about 120 mg, from about 100 mg to about 120 mg, from about 10 mg to about 100 mg, from about 20 mg to about 100 mg, from about 30 mg to about 100 mg, from about 40 mg to about 100 mg, from about 50 mg to about 100 mg, from about 60 mg to about 100 mg, from about 70 mg to about 100 mg, from about 80 mg to about 100 mg, or from about 90 mg to about 100 mg of the compound of formula (I), on a salt-free and anhydrous basis. In some embodiments, the compound of formula (I) is administered once daily to provide a total daily dosage of from about 50 mg to about 120 mg, from about 60 mg to about 120 mg, from about 70 mg to about 120 mg, from about 80 mg to about 120 mg, from about 90 mg to about 120 mg, from about 100 mg to about 120 mg, from about 50 mg to about 100 mg, from about 60 mg to about 100 mg, from about 70 mg to about 100 mg, from about 80 mg to about 100 mg, or from about 90 mg to about 100 mg of the compound of formula (I), on a salt-free and anhydrous basis. In some embodiments, the compound of formula (I) is administered once daily to provide a total daily dosage of about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, or about 200 mg of the compound of formula (I), on a salt-free and anhydrous basis. In some embodiments, the compound of formula (I) is administered once daily to provide a total daily dosage of about 10 mg, about 20 mg, about 30 mg, about 40 mg, about 50 mg, about 60 mg, about 70 mg, about 80 mg, about 90 mg, about 100 mg, about 110 mg, or about 120 mg of the compound of formula (I), on a salt-free and anhydrous basis. In some embodiments, the compound of formula (I) is administered once daily to provide a total daily dosage of about 50 mg, about 60 mg, about 70 mg, about 80 mg, about 90 mg, about 100 mg, about 110 mg, or about 120 mg of the compound of formula (I), on a salt-free and anhydrous basis.

In some embodiments, the compound of formula (I) is administered twice daily to provide a total daily dosage of no more than about 2000 mg of the compound of formula (I). In some embodiments, the compound of formula (I) is administered twice daily to provide a total daily dosage of from about 10 mg to about 120 mg, from about 20 mg to about 120 mg, from about 30 mg to about 120 mg, from about 40 mg to about 120 mg, from about 50 mg to about 120 mg, from about 60 mg to about 120 mg, from about 70 mg to about 120 mg, from about 80 mg to about 120 mg, from about 90 mg to about 120 mg, from about 100 mg to about 120 mg, from about 10 mg to about 100 mg, from about 20 mg to about 100 mg, from about 30 mg to about 100 mg, from about 40 mg to about 100 mg, from about 50 mg to about 100 mg, from about 60 mg to about 100 mg, from about 70 mg to about 100 mg, from about 80 mg to about 100 mg, or from about 90 mg to about 100 mg of the compound of formula (I), on a salt-free and anhydrous basis. In some embodiments, the compound of formula (I) is administered twice daily to provide a total daily dosage of from about 50 mg to about 120 mg, from about 60 mg to about 120 mg, from about 70 mg to about 120 mg, from about 80 mg to about 120 mg, from about 90 mg to about 120 mg, from about 100 mg to about 120 mg, from about 50 mg to about 100 mg, from about 60 mg to about 100 mg, from about 70 mg to about 100 mg, from about 80 mg to about 100 mg, or from about 90 mg to about 100 mg of the compound of formula (I), on a salt-free and anhydrous basis. In some embodiments, the compound of formula (I) is administered twice daily to provide a total daily dosage of about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, or about 200 mg of the compound of formula (I), on a salt-free and anhydrous basis. In some embodiments, the compound of formula (I) is administered twice daily to provide a total daily dosage of about 10 mg, about 20 mg, about 30 mg, about 40 mg, about 50 mg, about 60 mg, about 70 mg, about 80 mg, about 90 mg, about 100 mg, about 110 mg, or about 120 mg of the compound of formula (I), on a salt-free and anhydrous basis. In some embodiments, the compound of formula (I) is administered twice daily to provide a total daily dosage of about 50 mg, about 60 mg, about 70 mg, about 80 mg, about 90 mg, about 100 mg, about 110 mg, or about 120 mg of the compound of formula (I), on a salt-free and anhydrous basis.

In some embodiments, the compound of formula (I) is administered once daily. In some embodiments, the compound of formula (I) is administered once daily during each of one or more treatment cycles, as described herein. In some embodiments, the compound of formula (I) is administered twice daily. In some embodiments, the compound of formula (I) is administered twice daily during each of one or more treatment cycles, as described herein. In some embodiments, the compound of formula (I) is administered twice daily with a first dose and a second dose that are administered at least 10 hours apart, such as at least about 12 hours apart.

The compound of formula (I) can be administered to a subject under a fasted condition (e.g., after an overnight fast (minimum 10 hours) followed by 4 hours of fasting after the dose is taken). The subject is allowed to have water before and after the administration and the subject is given with water at the administration. In some embodiments, the compound of formula (I) is administered to the subject under a fasted condition. In some embodiments, the compound of formula (I) is administered to the subject under a fasted condition, wherein the subject has no food consumption except for water at least about 10 hours prior to the administration and at least about 4 hours post the administration.

The compound of formula (I) can be administered to a subject under a fed condition (e.g., after an overnight fast (minimum 10 hours) followed by a standardized high-calorie (approximately 800 to 1000 calories) breakfast, and the dose is taken in about 30 minutes after the start of the meal). In some embodiments, the compound of formula (I) is administered to the subject under a fed condition. In some embodiments, the compound of formula (I) is administered to the subject under a fed condition, wherein the subject has no food consumption except for water at least about 10 hours prior to the administration; and the compound of formula (I) is administered right after food intake.

The compound of formula (I) can be administered under a hybrid condition including the fasted and fed conditions as described herein. In some embodiments, the compound of formula (I) is administered under a hybrid condition including the fasted and fed conditions as described herein. In some embodiments, the compound of formula (I) is administered under a hybrid condition including a fasted condition and a fed condition, wherein, under the fasted condition, the subject has no food consumption except for water at least about 10 hours prior to the administration and at least about 4 hours post the administration; and under the fed condition, the subject has no food consumption except for water at least about 10 hours prior to the administration and the compound of formula (I) is administered right after food.

### III-5: Efficacy

A First-in-human, Randomized, Placebo-Controlled, Single and Multiple Ascending Dose Study can evaluate the safety, tolerability, pharmacokinetics, and pharmacodynamics of Compound (I) in subjects with propionic acidemia (PA) or methylmalonic acidemia (MMA), as described in Example 4.

Administration of a therapeutically effective amount of the compound of formula (I) may reduce or substantially eliminate one or more symptoms associated with PA and/or MMA in subjects. Administration of a therapeutically effective amount of the compound of formula (I) may reduce or substantially normalize one or more elevated biomarkers (e.g., MCA, MCA:citrate ratio, MMA, 3HP, propionylglycine, tiglylglycine, C3-carnitine, C3:C2-camitine ratio, ammonium, pantothenate, tricyclic acid (TCA) cycle intermediates, FGF21, and/or BNP) in the subject having PA and/or MMA. Administration of a therapeutically effective amount of the compound of formula (I) may substantially normalize one or more elevated biomarkers (e.g., lactate, blood gas, and/or amino acids) in the subject having PA and/or MMA. Administration of a therapeutically effective amount of the compound of formula (I) may increase or substantially normalize an acetyl-CoA level in the subject having PA and/or MMA.

In some embodiments, administration of a therapeutically effective amount of the compound of formula (I) reduces or substantially eliminates one or more symptoms associated with PA and/or MMA in subjects. In some embodiments, the one or more symptoms are selected from the group consisting of poor feeding, loss of appetite, failure to thrive (e.g., failure to grow and gain weight at an expected rate), emesis (vomiting), hypotonia (weak muscle tone), lethargy (lack of energy), listlessness, delayed development, intellectual disability, fever, infection, constipation, dehydration, acidosis, encephalopathy (brain damage), vision problems, pancreatitis, osteoporosis, renal failure, anemia (reduced red blood cells), leukopenia (reduced white blood cells), thrombocytopenia (reduced platelets), pancytopenia (reduced cells of various types), immune deficiency, bleeding problems, acidosis, heart abnormalities, heart failure (cardiomyopathy), heart rhythm problems (prolonged QTc interval), coma, stroke, seizures, respiratory distress, enlarged liver, dehydration, kenonemia (elevated acetone in blood), ketonuria (elevated acetone in urine), hyperammonemia (elevated ammonia in blood), hyperglycinemia (elevated glycine in blood), hyperglycinuria (elevated glycine in urine), hypoglycemia (low blood sugar), and a combination thereof. In some embodiments, the one or more symptoms are selected from the group consisting of poor feeding, loss of appetite, failure to thrive, emesis, hypotonia, lethargy, listlessness, delayed development, intellectual disability, fever, infection, constipation, dehydration, acidosis, encephalopathy, vision problems, pancreatitis, osteoporosis, renal failure, anemia, leukopenia, thrombocytopenia, pancytopenia, immune deficiency, bleeding problems, acidosis, heart abnormalities, heart failure, heart rhythm problems, coma, stroke, seizures, and a combination thereof. In some embodiments, the one or more symptoms are selected from the group consisting of respiratory distress, enlarged liver, lethargy, failure to thrive, emesis, acidosis, dehydration, hypotonia, developmental delay, intellectual disability, seizures, kenonemia, ketonuria, hyperammonemia, hyperglycinemia, hyperglycinuria, anemia, leukopenia, thrombocytopenia, pancytopenia, hypoglycemia, and a combination thereof.

In some embodiments, administration of a therapeutically effective amount of the compound of formula (I) reduces or substantially normalizes an elevated level of a biomarker selected from the group consisting of 2-methylcitrate or 2-methylcitric acid (MCA), MCA:citrate ratio, methylmalonate or methylmalonic acid (MMA), 3-hydroxypropionate or 3-hydroxypropionic acid (3HP), propionylglycine, tiglylglycine, C3-carnitine, C3:C2-carnitine ratio, ammonium, Fibroblast growth factor 21 (FGF21), and a combination thereof.

In some embodiments, administration of a therapeutically effective amount of the compound of formula (I) reduces or substantially normalizes an elevated 2-methylcitrate or 2-methylcitric acid (MCA) and/or an elevated ratio of MCA:citrate. In some embodiments, administration of a therapeutically effective amount of the compound of formula (I) reduces or substantially normalizes an elevated 2-methylcitrate or 2-methylcitric acid (MCA) and/or an elevated ratio of MCA:citrate plasma. In some embodiments, administration of a therapeutically effective amount of the compound of formula (I) reduces or substantially normalizes an elevated MCA. In some embodiments, administration of a therapeutically effective amount of the compound of formula (I) reduces or substantially normalizes an elevated MCA in plasma. In some embodiments, administration of a therapeutically effective amount of the compound of formula (I) reduces or substantially normalizes an elevated MCA:citrate ratio. In some embodiments, administration of a therapeutically effective amount of the compound of formula (I) reduces or substantially normalizes an elevated MCA:citrate ratio in plasma. In some embodiments, administration of a therapeutically effective amount of the compound of formula (I) reduces or substantially normalizes both elevated MCA and MCA:citrate ratio. In some embodiments, administration of a therapeutically effective amount of the compound of formula (I) reduces or substantially normalizes both elevated MCA and MCA:citrate ratio in plasma.

In some embodiments, administration of a therapeutically effective amount of the compound of formula (I) reduces or substantially normalizes an elevated methylmalonate or methylmalonic acid (MMA). In some embodiments, administration of a therapeutically effective amount of the compound of formula (I) reduces or substantially normalizes an elevated MMA in plasma.

In some embodiments, administration of a therapeutically effective amount of the compound of formula (I) reduces or substantially normalizes an elevated 3-hydroxypropionate or 3-hydroxypropionic acid (3HP). In some embodiments, administration of a therapeutically effective amount of the compound of formula (I) reduces or substantially normalizes an elevated 3HP in plasma.

In some embodiments, administration of a therapeutically effective amount of the compound of formula (I) reduces or substantially normalizes an elevated propionylglycine and/or tiglylglycine. In some embodiments, administration of a therapeutically effective amount of the compound of formula (I) reduces or substantially normalizes an elevated propionylglycine. In some embodiments, administration of a therapeutically effective amount of the compound of formula (I) reduces or substantially normalizes an elevated tiglylglycine. In some embodiments, administration of a therapeutically effective amount of the compound of formula (I) reduces or substantially normalizes both elevated propionylglycine and tiglylglycine.

In some embodiments, administration of a therapeutically effective amount of the compound of formula (I) reduces or substantially normalizes an elevated propionylcamitine (C3-camitine) level and/or an elevated C3:C2-carnitine ratio. In some embodiments, administration of a therapeutically effective amount of the compound of formula (I) reduces or substantially normalizes an elevated C3-camitine level and/or an elevated C3:C2-carnitine ratio in plasma. In some embodiments, administration of a therapeutically effective amount of the compound of formula (I) reduces or substantially normalizes an elevated C3-carnitine. In some embodiments, administration of a therapeutically effective amount of the compound of formula (I) reduces or substantially normalizes an elevated C3-carnitine in plasma. In some embodiments, administration of a therapeutically effective amount of the compound of formula (I) reduces or substantially normalizes an elevated C3:C2-carnitine ratio. In some embodiments, administration of a therapeutically effective amount of the compound of formula (I) reduces or substantially normalizes an elevated C3:C2-carnitine ratio in plasma. In some embodiments, administration of a therapeutically effective amount of the compound of formula (I) reduces or substantially normalizes both elevated C3-carnitine and C3:C2-carnitine ratio. In some embodiments, administration of a therapeutically effective amount of the compound of formula (I) reduces or substantially normalizes both elevated C3-carnitine and C3:C2-carnitine ratio in plasma.

In some embodiments, administration of a therapeutically effective amount of the compound of formula (I) reduces or substantially normalizes an elevated ammonium. In some embodiments, administration of a therapeutically effective amount of the compound of formula (I) reduces or substantially normalizes an elevated ammonium in plasma.

In some embodiments, administration of a therapeutically effective amount of the compound of formula (I) reduces or substantially normalizes an elevated Fibroblast growth factor 21 (FGF21). In some embodiments, administration of a therapeutically effective amount of the compound of formula (I) reduces or substantially normalizes an elevated FGF21 in plasma.

In some embodiments, administration of a therapeutically effective amount of the compound of formula (I) reduces or substantially normalizes an elevated pantothenate.

In some embodiments, administration of a therapeutically effective amount of the compound of formula (I) increase or substantially normalizes a reduced acetyl-CoA level.

Administration of a therapeutically effective amount of the compound of formula (I) can reduce or substantially normalize other elevated biomarkers, for example an elevated level of tricyclic acid (TCA) cycle intermediates (e.g., citrate, ketoglutarate, succinate, malate, etc.) and/or B-type natriuretic peptide (BNP). Administration of a therapeutically effective amount of the compound of formula (I) can substantially normalize levels of lactate, blood gas, and/or amino acids.

In some embodiments, administration of a therapeutically effective amount of the compound of formula (I) reduces or substantially normalizes an elevated level of one or more TCA cycle metabolites. In some embodiments, the one or more TCA cycle metabolites are selected from the group consisting of α-ketoglutarate, citrate, fumarate, isocitrate, malate, methylcitrate, methylmalonate, oxaloacetate, succinate, glucose, glycerate, phenylpyruvate, phosphoenolpyruvate (PEP), lactate, glucosamine, choline, creatinine, and creatine. In some embodiments, the one or more TCA cycle metabolites are selected from the group consisting of α-ketoglutarate, citrate, isocitrate, malate, methyleitrate, methylmalonate, oxaloacetate, succinate, glucose, glycerate, phosphoenolpyruvate (PEP), and choline. In some embodiments, the one or more TCA cycle metabolites are selected from the group consisting of α-ketoglutarate, malate, methylcitrate, methylmalonate, oxaloacetate, and succinate. In some embodiments, the one or more TCA cycle metabolites are selected from the group consisting of α-ketoglutarate, citrate, succinate, and malate. In some embodiments, the one or more TCA cycle metabolites include malate. In some embodiments, administration of a therapeutically effective amount of the compound of formula (I) reduces or substantially normalizes an elevated malate level. In some embodiments, the levels of TCA cycle metabolites of the one or more TCA cycle metabolites are urinary levels. In some embodiments, the levels of TCA cycle metabolites of the one or more TCA cycle metabolites are plasma levels.

In some embodiments, administration of a therapeutically effective amount of the compound of formula (I) reduces or substantially normalizes elevated malate, methylcitrate, citrate, α-ketoglutarate, succinate, glycine, and methylmalonate levels in urine and/or plasma. In some embodiments, administration of a therapeutically effective amount of the compound of formula (I) reduces or substantially normalizes elevated malate, citrate, α-ketoglutarate, and succinate in urine and/or plasma.

In some embodiments, administration of a therapeutically effective amount of the compound of formula (I) reduces or substantially normalizes an elevated B-type natriuretic peptide (BNP).

In some embodiments, administration of a therapeutically effective amount of the compound of formula (I) substantially normalizes levels of lactate, blood gas, and/or amino acids, which are abnormal prior to the treatment.

In some embodiments, the subject is further evaluated to by one or more tests (e.g., tests according to Table 6, Table 7, Table 8, and Table 9 to provide overall assessments including plasma pharmacokinetic and/or pharmacodynamic profiles. Examples of such tests are described in, e.g., Table 6, Table 7, Table 8, and Table 9 of Example 4.

In some embodiments, the subject is further evaluated for one or more biomarkers to determine a correlation of the one or more biomarkers to a response to the treatment of PA and/or MMA. Examples of such evaluation are described in Table 6, Table 7, Table 8, and Table 9 of Example 4.

### III-6: Oral Dosage Form

The oral dosage form including the compound of formula (I) can be in any oral dosage forms including one or more pharmaceutically acceptable carriers and/or excipients. Oral preparations include tablets, pills, powder, dragees, capsules, liquids, lozenges, cachets, gels, syrups, slurries, suspensions, etc., suitable for ingestion by the patient.

For preparing oral dosage forms including the compound of formula (I), pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton PA (“Remington's”).

In powders, the carrier is a finely divided solid, which is in a mixture with the finely divided active component. In tablets, the active component is mixed with the carrier having the necessary binding properties in suitable proportions and compacted in the shape and size desired.

The powders, capsules and tablets preferably contain from 5% or 10% to 70% of the active compound. Suitable carriers are magnesium carbonate, magnesium stearate, tale, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other excipients, is surrounded by a carrier, which is thus in association with it. Similarly, cachets and lozenges are included. Tablets, powders, capsules, pills, cachets, and lozenges can be used as solid dosage forms suitable for oral administration.

Suitable solid excipients include, but are not limited to, magnesium carbonate; magnesium stearate; talc; pectin; dextrin; starch; tragacanth; a low melting wax; cocoa butter; carbohydrates; sugars including, but not limited to, lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins including, but not limited to, gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the dosage forms can also be used orally using, for example, push-fit capsules made of gelatin, as well as soft, sealed capsules made of gelatin and a coating such as glycerol or sorbitol. Push-fit capsules can contain the compound of formula (I) mixed with a filler or binders such as lactose or starches, lubricants such as talc or magnesium stcarate, and, optionally, stabilizers. In soft capsules, the compound of formula (I) may be dissolved or suspended in suitable liquids, such as fatty oils, liquid paraffin, or liquid polyethylene glycol with or without stabilizers.

For preparing suppositories, a low melting wax, such as a mixture of fatty acid glycerides or cocoa butter, is first melted and the compound of formula (I) are dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions.

Aqueous solutions suitable for oral use can be prepared by dissolving the compound of formula (I) in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending the compound of formula (I) in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J Pharmacol. Exp. Ther. 281:93-102, 1997. The pharmaceutical formulations including the compound of formula (I) can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

### IV. Methods of Treating Pkan

In a second aspect, the present disclosure provides a method of treating Pantothenate Kinase Associated Neurodegeneration (PKAN). The method includes administering to a subject in need thereof a therapeutically effective amount of a compound represented by formula (I):

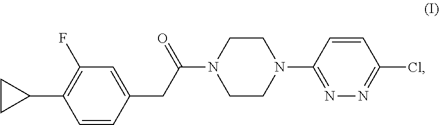 [SMILES: O=C(Cc1ccc(C2CC2)c(F)c1)N1CCN(c2ccc(Cl)nn2)CC1]

or a pharmaceutically acceptable salt, hydrate, solvate, or a combination thereof.

In some embodiments, the method includes administering to a subject in need thereof a therapeutically effective amount of the compound represented by formula (I), a pharmaceutically acceptable salt thereof.

### IV-1: Compound of Formula (I)

The compound of formula (I) is described in Section III-1. In some embodiments, the compound of formula (I) is any one of embodiments as described in Section III-1.

In some embodiments, the compound of formula (I) is represented by the formula:

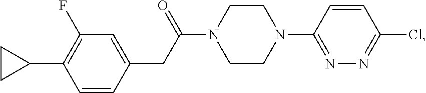 [SMILES: O=C(Cc1ccc(C2CC2)c(F)c1)N1CCN(c2ccc(Cl)nn2)CC1]

having the name of 1-(4-(6-chloropyridazin-3-yl)piperazin-1-yl)-2-(4-cyclopropyl-3-fluorophenyl)ethan-1-one.

### IV-2: Subject

In some embodiments, the subject has Pantothenate Kinase Associated Neurodegeneration (PKAN). In some embodiments, the subject has a documented diagnosis of PKAN as indicated by a confirmed mutation in the pantothenate kinase 2 (PANK2) gene.

The subject can have one or more symptoms of PKAN. In some embodiments, the subject has one or more symptoms selected from abnormality of extrapyramidal motor function, akinesia, muscular rigidity, chorca, athetosis, akathisia, abnormality of eye movement, acanthocytosis, attention deficit hyperactivity disorder (ADHD), behavioral abnormality, blindness, bulbar signs, Bull's eye maculopathy, choreoathetosis, craniofacial dystonia, dementia, depressivity, dysarthria, dystonia, emotional lability, tendency to fracture bones, gait disturbance, global developmental delay, hyperreflexia, impaired convergence, impulsivity, intellectual disability, intention or kinetic tremor, iron accumulation in globus pallidus and/or substantia nigra, limb dystonia, limb pain, loss of ability to walk, mental deterioration, neurological speech impairment, nyctalopia, obsessive-compulsive behavior, optic atrophy, osteopenia, palilalia, pallidal degeneration, parkinsonism, peripheral visual field loss, pigmentary retinopathy, psychotic mentation, retinal degeneration, retinal flecks, rigidity, rod-cone dystrophy, saccadic smooth pursuit, seizures, slurred speech, spasticity, speech articulation difficulties, tics, tip-toe gait, toe extensor amyotrophy, visual field defect, and visual impairment. In some embodiments, the subject has one or more symptoms selected from abnormality of extrapyramidal motor function, choreoathetosis, dystonia, gait disturbance, loss of ability to walk, retinal degeneration, spasticity, and visual impairment. In some embodiments, the subject has dystonia. In some embodiments, the subject was diagnosed with dystonia before the age of 10.

The subject can be a pediatric subject or an adult subject. In some embodiments, the subject is at least about 6 years of age. In some embodiments, the subject is at least about 10 years of age. In some embodiments, the subject is at least about 12 years of age. In some embodiments, the subject is at least about 16 years of age. In some embodiments, the subject is at least about 18 years of age. In some embodiments, the subject is between 6-18 years old. In some embodiments, the subject is between 6-12 years old. In some embodiments, the subject is between 12-15 years old. In some embodiments, the subject is between 16-18 years old.

In some embodiments, the subject has a therapeutic regimen for PKAN disease management. In some embodiments, the subject is taking or has previously taken a therapy selected from fosmetpantotenate, pantothenate (e.g., calcium pantothenate), deferiprone, baclofen, a muscle relaxant (anticholinergics, benzodiazepines, and other anti-spasticity agents), and/or a treatment for Parkinson's disease. In some embodiments, the subject has previously undergone a surgical ablation procedure such as a thalamotomy or pallidotomy. In some embodiments, the subject is being treated with or has previously undergone treatment with deep brain stimulation (DBS).

In some embodiments, the subject has abnormal levels of one or more markers selected from the group consisting of neurofilament light chain (NfL), glial fibraillary acid protein (GFAP), ubiquitin carboxyl-terminal hydrolase La (UCH-L1), and Tau. In some embodiments, the subject has elevated levels of one or more markers selected from the group consisting of neurofilament light chain (NfL), glial fibraillary acid protein (GFAP), ubiquitin carboxyl-terminal hydrolase La (UCH-L1), and Tau (e.g., in serum). In some embodiments, the subject has depressed levels of one or more markers selected from the group consisting of neurofilament light chain (NfL), glial fibraillary acid protein (GFAP), ubiquitin carboxyl-terminal hydrolase La (UCH-L1), and Tau (e.g., in serum). In some embodiments, the subject has elevated levels of Tau in serum.

In some embodiments, the subject has an elevated pantothenate level. In some embodiments, the subject has an elevated pantothenate level in plasma.

In some embodiments, the subject has a reduced acetyl-CoA level. In some embodiments, the subject has a reduced acetyl-CoA level in whole blood.

In some embodiments, the subject does not require concurrent use of a strong or moderate CYP3A4 inhibitor. In some embodiments, the subject does not require concurrent use of a strong CYP2C19 inhibitor.

In some embodiments, the subject having PKAN does not have one or more conditions selected from the group consisting of:
a) alanine aminotransferase (ALT) and/or aspartate aminotransferase (AST) exceed two times of a upper limit of normal (ULN);
b) total bilirubin exceeds two times of a upper limit of normal (ULN);
c) an international normalized ratio (INR) is more than 1.4;
d) a baseline estimated glomerular filtration rate (eGFR) is less than 45 mL/min calculated using the CKD-EPI formula;
e) a positive test result for hepatitis B surface antigen, hepatitis C virus antibody, or HIV types 1 or 2 antibodies; and
f) a positive test result for SARS-CoV-2.

Further inclusion and exclusion criteria for subjects who may benefit from treatment with a compound of formula (I), such as subjects enrolled in a First-in-human, Randomized, Placebo-Controlled, and/or Single and Multiple Ascending Dose Study or subjects enrolled in a Ramonized, Double-blind, Placebo-controlled Study to Evaluate the Efficacy, Safety, and Tolerability of Compound (I) in Patients with PKAN, are described in Examples 4 and 7.

In some embodiments, the subject meets all of inclusion criteria of 1) to 9) and 12) to 16) as described in Example 4. In some embodiments, the subject meets all of inclusion criteria of 1) to 9) and 12) to 16) as described in Example 4, provided that the subject does not meet any one of exclusion criteria of 1) to 23) as described in Example 4. In some embodiments, the subject meets all of the inclusion criteria as described in Example 7. In some embodiments, the subject meets all of the inclusion criteria as described in Example 7, provided that the subject does not meet any of the exclusion criteria as described in Example 7.

In some embodiments, the subject is a mammal. In some embodiments, the subject is human.

### IV-3: Treatment Cycle and Dose Adjustment

The treatment cycle and dose adjustment is described in Section III-3. In some embodiments, the treatment cycle and dose adjustment is any one of embodiments as described in Section III-3.

The dose escalation or de-escalation can be determined by a dose-limiting toxicity (DLT) assessment, based on accepted criteria of Example 4 (as described in Section III-3) and/or Example 7. In some embodiments, the administration of the compound of formula (I) includes a dose escalation after a previous treatment cycle, if safety assessment meets accepted criteria of Example 4 and/or Example 7. In some embodiments, the administration of the compound of formula (I) includes a dose escalation in a second treatment after a first treatment cycle if safety assessment meets accepted criteria of Example 4 and/or Example 7. In some embodiments, the administration of the compound of formula (I) includes a dose escalation in a third treatment after a second treatment cycle if safety assessment meets accepted criteria of Example 4 and/or Example 7. In some embodiments, the administration of the compound of formula (I) includes a dose escalation in a fourth treatment after a third treatment cycle if safety assessment meets accepted criteria of Example 4 and/or Example 7. In some embodiments, the administration of the compound of formula (I) includes a dose escalation in a fifth treatment after a fourth treatment cycle if safety assessment meets accepted criteria of Example 4 and/or Example 7. In some embodiments, the administration of the compound of formula (I) includes a dose escalation in a sixth treatment after a fifth treatment cycle if safety assessment meets accepted criteria of Example 4 and/or Example 7. In some embodiments, the administration of the compound of formula (I) includes a dose de-escalation in a third treatment after a second treatment cycle according to the criteria of Example 4 and/or Example 7. In some embodiments, the administration of the compound of formula (I) includes a dose de-escalation in a fourth treatment after a third treatment cycle according to the criteria of Example 4 and/or Example 7. In some embodiments, the administration of the compound of formula (I) includes a dose de-escalation in a fifth treatment after a fourth treatment cycle according to the criteria of Example 4 and/or Example 7. In some embodiments, the administration of the compound of formula (I) includes a dose de-escalation in a sixth treatment after a fifth treatment cycle according to the criteria of Example 4 and/or Example 7. In some embodiments, the administration of the compound of formula (I) includes a dose de-escalation within a treatment cycle, according to the criteria of Example 4 and/or Example 7.

### IV-4: Therapeutically Effective Amount/Administration

The therapeutically effective amount/administration is described in Section III-4. In some embodiments, the therapeutically effective amount/administration is any one of embodiments as described in Section III-4.

### IV-5: Efficacy

A First-in-human, Randomized, Placebo-Controlled, Single and Multiple Ascending Dose Study can evaluate the safety, tolerability, pharmacokinetics, and pharmacodynamics of Compound (I) in subjects with propionic acidemia (PA) or methylmalonic acidemia (MMA), as described in Example 4. This study can also inform safe and potentially efficacious dosing for pantothenate kinase-associated neurodegeneration (PKAN), as described in Example 7.

Administration of a therapeutically effective amount of the compound of formula (I) may reduce or substantially eliminate one or more symptoms associated with PKAN in subjects. Administration of a therapeutically effective amount of the compound of formula (I) may reduce or substantially normalize one or more elevated biomarkers (e.g., pantothenate or tau) in the subject having PKAN. Administration of a therapeutically effective amount of the compound of formula (I) may increase or substantially normalize an acetyl-CoA level in the subject having PKAN.

In some embodiments, administration of a therapeutically effective amount of the compound of formula (I) reduces or substantially eliminates one or more symptoms associated with PKAN in subjects. In some embodiments, the one or more symptoms are selected from the group consisting of abnormality of extrapyramidal motor function, akinesia, muscular rigidity, chorea, athetosis, akathisia, abnormality of eye movement, acanthocytosis, attention deficit hyperactivity disorder (ADHD), behavioral abnormality, blindness, bulbar signs, Bull's eye maculopathy, choreoathetosis, craniofacial dystonia, dementia, depressivity, dysarthria, dystonia, emotional lability, tendency to fracture bones, gait disturbance, global developmental delay, hyperreflexia, impaired convergence, impulsivity, intellectual disability, intention or kinetic tremor, iron accumulation in globus pallidus and/or substantia nigra, limb dystonia, limb pain, loss of ability to walk, mental deterioration, neurological speech impairment, nyctalopia, obsessive-compulsive behavior, optic atrophy, osteopenia, palilalia, pallidal degeneration, parkinsonism, peripheral visual field loss, pigmentary retinopathy, psychotic mentation, retinal degeneration, retinal flecks, rigidity, rod-cone dystrophy, saccadic smooth pursuit, seizures, slurred speech, spasticity, speech articulation difficulties, tics, tip-toe gait, toe extensor amyotrophy, visual field defect, visual impairment, and a combination thereof. In some embodiments, the one or more symptoms are selected from the group consisting of abnormality of extrapyramidal motor function, choreoathetosis, dystonia, gait disturbance, loss of ability to walk, retinal degeneration, spasticity, and visual impairment, and a combination thereof. In some embodiments, the one or more symptoms include dystonia. In some embodiments, the subject was diagnosed with dystonia before the age of 10.

In some embodiments, administration of a therapeutically effective amount of the compound of formula (I) reduces or substantially normalizes an elevated pantothenate in at least one of plasma, serum, and dried blood spot, such as in plasma.

In some embodiments, administration of a therapeutically effective amount of the compound of formula (I) increases or substantially normalizes a reduced acetyl-CoA level in at least one of plasma, serum, and dried blood spot, such as in plasma.

In some embodiments, administration of a therapeutically effective amount of the compound of formula (I) increases, reduces, or substantially normalizes a level of a neural biomarker such as neurofilament light chain (NfL), glial fibraillary acid protein (GFAP), ubiquitin carboxyl-terminal hydrolase La (UCH-L1), and/or Tau in at least one of plasma, serum, and dried blood spot. In some embodiments, administration of a therapeutically effective amount of the compound of formula (I) reduces or substantially normalizes a level of Tau in at least one of plasma, serum, and dried blood spot, such as in serum.

In some embodiments, the subject is further evaluated to by one or more tests (e.g., tests according to Table 6, Table 7, Table 8, and Table 9 in Example 4, or tests described in Example 7) to provide overall assessments including plasma pharmacokinetic and/or pharmacodynamic profiles. Examples of such tests are described in, e.g., Table 6, Table 7, Table 8, and Table 9 of Example 4 and Table 15 in Example 7.

In some embodiments, the subject is further evaluated for one or more biomarkers to determine a correlation of the one or more biomarkers to a response to the treatment of PKAN. Examples of such evaluations are described in Table 6, Table 7, Table 8, and Table 9 of Example 4, and Table 15 in Example 7.

### IV-6: Oral Dosage Form

The oral dosage form is described in Section III-6. In some embodiments, the oral dosage form is any one of embodiments as described in Section III-6.

V. LIST OF ABBREVIATIONS

| Abbreviation | Definition |
|---|---|
| ΔHR | change from baseline for heart rate |
| ΔΔHR | placebo-corrected change from baseline for heart rate |
| ΔPR | change from baseline for PR interval |
| ΔΔPR | placebo-corrected change from baseline for PR interval |
| ΔQRS | change from baseline for QRS duration |
| ΔΔQRS | placebo-corrected change from baseline for QRS duration |
| ΔQTcF | change from baseline for QT interval corrected for heart |
| | rate using Fridericia's formula |
| ΔΔQTcF | placebo-corrected change from baseline for QT interval |
| | corrected for heart rate using Fridericia's formula |
| AE | adverse event |
| Ae₂₄ | amount of drug excreted in urine at 24 hours |
| Ae₄₈ | amount of drug excreted in urine at 48 hours |
| ALT | alanine aminotransferase |
| ANC | absolute neutrophil count |
| AST | aspartate aminotransferase |
| AUC | area under the plasma concentration versus time curve |
| AUC_{0-inf}/D | dose normalized area under the plasma concentration |
| | versus time curve from time 0 extrapolated to infinity |
| AUC₀₋₁₂ | area under the plasma concentration versus time curve |
| | from time 0 to 12 hours after dosing |
| AUC₀₋₈ | area under the plasma concentration versus time curve |
| | from time 0 to 8 hours after dosing |
| AUC₀₋₂₄ | area under the plasma concentration versus time curve |
| | from time 0 to 24 hours after dosing |
| AUC₀₋₄₈ | area under the plasma concentration versus time curve |
| | from time 0 to 48 hours after dosing |
| AUC_{0-inf} | area under the plasma concentration versus time curve |
| | from time 0 extrapolated to infinity |
| AUC₀₋ₜₐᵤ | area under the plasma concentration versus time curve |
| | from time 0 until the end of the dosing interval |
| AUCₗₐₛₜ | area under the plasma concentration versus time curve |
| | from time 0 to the last quantifiable concentration |
| BCRP | breast cancer resistance protein |
| BID | twice daily |
| BLQ | below the limit of quantification |
| BMI | body mass index |
| bpm | beats per minute |
| BSEP | bile salt export pump |
| ° C. | degrees Celsius |
| CFR | Code of Federal Regulations |
| CKD-EPI | chronic kidney disease epidemiology collaboration |
| Cₘₐₓ/D | dose normalized maximum observed plasma |
| CI | concentration confidence interval |
| CL/F | apparent total body clearance |
| CLr | renal clearance of drug |
| CL_{SS}/F | apparent total body clearance at steady state |
| Cₘₐₓ | maximum observed plasma concentration |
| CoA | coenzyme A |
| C-QTc | concentration-QTc |
| CSF | cerebrospinal fluid |
| C-SSRS | Columbia-Suicide Severity Rating Scale |
| CV | coefficient of variation |
| CYP | cytochrome P450 |
| DBS | dried blood spot |
| DLT | dose-limiting toxicity |
| ECG | electrocardiogram |
| ED | early discontinuation |
| eCRF | electronic case report form |
| eGFR | estimated glomerular filtration rate |
| EOS | end of study |
| FCC | Food Chemicals Codex |
| FDA | Food and Drug Administration |
| FE | food effect |
| FGF21 | Fibroblast growth factor 21 |
| FSH | follicle-stimulating hormone |
| g | gram |
| GLP | Good Laboratory Practice |
| GMCC | glyceryl monocaprylocaprate |
| HED | human equivalent dose |
| h | hour |
| HR | heart rate |
| ICF | informed consent form |
| ICH | International Council for Harmonisation |
| IND | investigational new drug |
| INR | international normalized ratio |
| IRB | institutional review board |
| JECFA | Joint FAO/WHO Expert Committee on Food Additives |
| JP | Japanese Pharmacopoeia |
| JPE | Japanese Pharmaceutical Excipients |
| JSFA | Japanese Standards for Food Additives |
| kg | kilogram |
| LC/MS/MS | Liquid chromatography/mass spectrometry/mass |
| | spectrometry |
| m/z | mass to charge ratio |
| MAD | multiple ascending dose |
| MATE | multidrug and toxic compound extrusion |
| MedDRA | Medical Dictionary for Regulatory Activities |
| mg | milligram |
| μL | microliter |
| μM | micromolar |
| min | minute |
| mm | millimeter |
| mM | millimolar |
| MMA | methylmalonic acidemia |
| MRSD | maximum recommended starting dose |
| mTorr | milliTorr |
| NDA | New drug application |
| nm | nanometer |
| NOAEL | no-observed-adverse-effect level |
| OAT | organic anion transporter |
| OATP | organic anion transporting polypeptide |
| OCT | organic cation transporter |
| P-gp | P-glycoprotein |
| PA | propionic acidemia |
| PAD | pharmacologically active dose |
| PD | pharmacodynamic(s) |
| PEG | polyethylene glycol |
| Ph Eur | European Pharmacopoeia |
| PK | pharmacokinetic(s) |
| pmol | picomolar |
| ppm | parts per million |
| psi | pounds per square inch |
| QTc | QT interval corrected for heart rate |
| QTcF | QT interval corrected for heart rate using Fridericia's |
| | formula |
| RAUC | accumulation ratio |
| SAD | single ascending dose |
| SAE | serious adverse event |
| SARS-CoV-2 | severe acute respiratory syndrome coronavirus 2 |
| SD | standard deviation |
| SF | sugar free |
| SOE | schedule of events |
| SRC | safety review committee |
| t_{1/2} | terminal elimination half-life |
| TCA | tricarboxylic acid |
| TEAE | treatment-emergent adverse event |
| Tₘₐₓ | time to maximum observed plasma concentration |
| ULN | upper limit of normal |
| USP-NF | United States Pharmacopeia and National Formulary |
| V | volts |
| V_{z}/F | apparent volume of distribution |
| Vz_{SS}/F | apparent volume of distribution at steady state |
| xg | times gravity |
| λₑₘ | emission wavelength |
| λₑₓ | excitation wavelength |

### VI. Examples

### Example 1: Efficacy of Compound (I) in Pcca^{-/-}PCCA(A138T)^{tg/0} Mice

An initial mouse model of propionic acidemia (PA) was developed via disruption of the murine Pcca gene (Pcca^{-/-} mice). Pcca^{-/-} mice lack PCCA protein, exhibit extremely high levels of C3-camitine and methylcitrate, and die within 36 hours of birth. Investigation of therapies for PA in this model is challenging due to the short life span of these animals.

To address these issues, a hypomorphic mouse model of PA was generated expressing a variant of the human PCCA protein that partially replaces the deletion in the Pcca^{-/-} mice allowing them to survive to adulthood. Pcca^{-/-}PCCA(A138T)^{tg/0} mice have limited PCC activity (2% of wild-type) and exhibit several hallmarks of human PA disease, including elevated plasma C3-carnitine, methylcitrate, glycine, alanine, lysine, and ammonia. Pcca^{-/-}PCCA(A138T)^{tg/0} mice also had increased heart mass compared to wild-type mice, similar to the cardiomyopathy observed in human PA patients, and exhibit decreased median lifespan. The fact that this mouse model recapitulates many of the hallmarks of PA in humans makes it an ideal model in which to investigate therapies for the treatment of PA.

Mouse Model: Pcca^{-/-}PCCA(A138T)^{tg/0} mice were obtained from Dr. Michael Barry's lab at the Mayo Clinic. The mice were genotyped using the mPcca and hPCCA primers listed in Table 1. Pcca^{+/-}(A138T)^{tg/0} were crossed with Pcca^{+/-} to maintain a single copy of the human cDNA transgene PCCA(A138T). Pcca heterozygotes were screened for the presence of the hPCCA(A138T) transgene with the hPCCA primers and GFP primers described in Table 1. The primer sets were used to detect the presence or absence of the mPcca gene, and to detect the presence or absence of the hPCCA(A138T) transgene with hPcca and green fluorescent protein (GFP) primers. The progeny followed a Mendelian inheritance pattern and 1 in 8 pups were the desired genotype Pcca^{-/-}PCCA(A138T)^{tg/0}.

TABLE 1
Primers and PCR conditions for genotyping
Pcca^{-/-} PCCA(A138T)^{tg/0} mice

| | Primer | Seqeunce | PCR |
|---|---|---|---|
| Gene | Name | 5′-3′ | conditions |
| mPCCA | mPCCA | AGGAAGCC | 94° C., 5 min + |
| | For2 | AGGAAAGG | 45 cycles |
| | | TTATTTG | (94° C., 30 sec + |
| | | (SEQ ID | 64° C., 30 sec + |
| | | NO.: 1) | 72° C., 30 sec) + |
| | mPCCA | CCCTACAG | 72° C., 2 min + |
| | Rev3 | GCAATTTC | 4° C., ∞ |
| | | TCCTC | WT = 457 bp |
| | | (SEQ ID | KO = 688 bp |
| | | NO.: 2) | |
| | mPCCA | GGATGATC | |
| | NeoRev4 | TGGACGAA | |
| | | GAGC | |
| | | (SEQ ID | |
| | | NO.: 3) | |
| hPCCA | hPTCCA | ACAGTGCT | 94º C., 5 min + |
| (A158T) | For | TAATGGTG | 40 cycles |
| | | TCCC | (94° C., 30 sec + |
| | | (SEQ ID | 60° C., 30 sec + |
| | | NO.: 4) | 72° C., 45 sec) + |
| | hPCCA | CTTCTGCA | 72° C., 2 min + |
| | Rev | TCCTTGAC | 4° C., ∞ |
| | | TACTCC | Positive = 485 bp |
| | | (SEQ ID | Int'l Control = |
| | | NO.: 5) | 200 bp |
| | oIMR0015 | CAAATGTT | |
| | | GCTTGTCT | |
| | | GGTG | |
| | | (SEQ ID | |
| | | NO.: 6) | |
| | oIMR0016 | GTCAGTCG | |
| | | AGTGCACA | |
| | | GTTT | |
| | | (SEQ ID | |
| | | NO.: 7) | |
| GFP | GFP-For1 | CACATGAA | 94° C., 5 min + |
| | | GCAGCACG | 40 cycles |
| | | ACTT | (94° C., 30 sec + |
| | | (SEQ ID | 58° C., 30 sec + |
| | | NO.: 8) | 72° C, 30 sec) + |
| | GFP-Rev1 | TGCTCAGG | 72° C., 2 min + |
| | | TAGTGGTT | 4° C., ∞ |
| | | GTCG | Positive = 380 bp |
| | | (SEQ ID | Int'l Control = |
| | | NO.: 9) | 200 bp |
| | oIMR0015 | CAAATGTT | |
| | | GCTTGTCT | |
| | | GGTG | |
| | | (SEQ ID | |
| | | NO.: 10) | |
| | oIMR0016 | GTCAGTCG | |
| | | AGTGCACA | |
| | | GTTT | |
| | | (SEQ ID | |
| | | NO.: 11) | |

The mice were maintained at 72′±2° F., humidity 50%±10% and a 14-hour light/10-hour dark cycle with the dark cycle starting at 20:00 hrs. Water was supplied ad libitum. The mice were randomized into the treatment arms to achieve a normal weight distribution. Compound (I) was formulated at 22.5 parts per million (ppm) (3 mg/kg) in chow (Envigo #TD170542) and Pcca^{-/-}PCCA(A138T)^{tg/0} animals were maintained on this diet±Compound (I) 30 days after weaning at P20. Assuming a wild-type mouse eats 4 grams (g)/day and weighs 30 g, 22.5 ppm in chow is approximately equivalent to 3 mg/kg in chow. However, this is merely an approximation, as Pcca^{-/-}PCCA(A138T)^{tg/0} mice weigh less but also eat less than wild-type mice. At P45, animals were acclimatized in metabolic cages for 24 hours and subsequently 24-hr urine samples were collected and stored frozen at −20° C. until analysis. Blood was collected in ethylenediaminetetraacetic acid (EDTA) at P50 by cardiac puncture from isoflurane-anesthetized animals, plasma was prepared and stored frozen at −80° C. until analysis. Liver, brain, and heart were quickly excised, immediately flash frozen in liquid nitrogen and stored frozen at −80° C. until analysis. The tissue samples were used for total CoA, acyl-CoA, acyl camitine, and Compound (I) determinations

Compound (I) Extraction and Quantification by LC/MS/MS: Plasma (20 μL) was added to 100 μL acetonitrile containing 0.6 μM warfarin to a final concentration of 0.5 μM. The samples were incubated on ice for 30 min. Samples were spun at 3500×g for 10 min to pellet debris, and the supernatant was transferred to a glass vial. A Compound (I) standard curve was created by spiking in known concentrations of Compound (I) into 20 μL of plasma from a control mouse and following the above procedure. 30 mg of tissue was homogenized in 2 mL of 80% methanol containing 0.1 μM warfarin and incubated at −80° C. for 4 hr. Samples were spun at 3500×g for 10 min to pellet debris, supernatant was transferred to a glass tube and dried down using a Savant SPD1010 Speed-Vac (Thermo Scientific) overnight. Samples were resuspended in 400 μL of 80% acetonitrile to a final concentration of 0.5 μM and transferred to a glass vial.

Compound (I) was analyzed using a Shimadzu Prominence UFLC attached to a QTrap 4500 equipped with a Turbo V ion source (Sciex). Samples (5 μL) were injected onto an XSelect® HSS C18, 2.5 μm, 3.0×150 mm column (Waters) using a flow rate of 0.25 mL/min. Solvent A was 0.1% formic acid in water, and Solvent B was acetonitrile with 0.1% formic acid. The HPLC program was the following: starting solvent mixture of 50% B, 0 to 0.5 min isocratic with 50% B; 0.5 to 1.5 min linear gradient to 95% B; 1.5 to 20 min isocratic with 95% B; 20 to 21 min linear gradient to 50% B; 21 to 25 min isocratic with 50% B. The QTrap 4500 was operated in the positive mode, and the ion source parameters were: ion spray voltage, 5500 V; curtain gas, 30 psi; temperature, 450° C.; collision gas, medium; ion source gas 1, 30 psi; and ion source gas 2, 40 psi. The MRM transition for Compound (I) was 375.2/199.1 n/z and for warfarin was 309.1/163.0 m/z both with a declustering potential, 65 V and collision energy, 30 V. The system was controlled by the Analyst® software (Scicx) and analyzed with MultiQuant™ 3.0.2 software (Sciex).

Tissue Total CoA determinations: Either brain or liver tissue (30-40 mg) was homogenized in 1 mM potassium hydroxide (KOH) to hydrolyze all the acyl chains and then the CoA was derivatized with monobromobimane (mBBr). The CoA bimane was purified on a SPE 2-(2-pyridyl) ethyl column and analyzed by HPLC with fluorescent detection λₑₓ=393 nm, λₑₘ=470 nm. The retention time was determined by running a CoA standard before each set of samples. Total CoA was quantified using a standard curve generated with known amounts of CoA.

Acyl-CoA Measurements by Mass Spectrometry: Acyl-CoAs were quantified as described by Sharma et al., Nat. Commun. 2018; 9:4399. Briefly, 30-40 mg of tissue was homogenized in 2 mL methanol and 1 mL water and incubated on ice for 30 minutes. 1.5 mL of chloroform and 1.2 mL of water was added to remove the lipids. The aqueous top layer was collected and 30 pmol of [13C]acetyl-CoA (Sigma) was added and dried overnight in a speedvac.

Mass spectrometry of the acyl-CoA distribution was performed using a Finnigan TSQ Quantum (Thermo Electron) triple-quadrupole mass spectrometer. The instrument was operated in positive mode using single ion monitoring (SIM) neutral loss scanning corresponding to the loss of the phosphoadenosine diphosphate from CoA species. The ion source parameters were as follows: spray voltage, 4,000 V; capillary temperature, 250° C.; capillary offset, −35 V; sheath gas pressure, 10; auxiliary gas pressure, 5; and tube lens offset was set by infusion of the polytyrosine tuning and calibration in electrospray mode. Acquisition parameters were as follows: scan time, 0.5 s; collision energy, 30 V; peak width Q1 and Q3, 0.7 FWHM; Q2 CID gas, 0.5 mTorr; source CID, 10 V; neutral loss, 507.0 m/z; SIM mass of 810 m/z with a scan width of 8 m/z to capture the signal from cellular acetyl-CoA and the [13C]acetyl-CoA internal standard.

Metabolites from Urine and Plasma: Water was added to thawed 24-hr urine samples to a final volume of 1 mL for each mouse. To 100 μL of urine or 20 μL of plasma, warfarin standard was added at a final concentration of 0.2 μM and the sample was incubated at −80° C. for 1 hour. The sample was centrifuged at 6000×g for 10 minutes to remove precipitants and the supernatant was analyzed by LC/MS/MS. Analysis was performed using a Shimadzu Prominence UFLC attached to a QTrap 4500 equipped with a Turbo V ion source (Sciex). Samples (5 μL) were injected onto an XSelect® HSS C18, 2.5 m, 3.0×150 mm column (Waters) using a flow rate of 0.4 mL/min. Solvent A was 0.1% formic acid in water, and Solvent B was acetonitrile+0.1% formic acid. The HPLC program was the following: starting solvent mixture of 25% B, 0 to 2 min isocratic with 25% B; 2 to 10 min linear gradient to 100% B; 10 to 20 min isocratic with 100% B; 20 to 22 min linear gradient to 25% B; 22 to 25 min isocratic with 25% B. The QTrap 4500 was operated in the negative mode, and the ion source parameters were: ion spray voltage, −4500 V; curtain gas, 40 psi; temperature, 500° C.; collision gas, medium; ion source gas 1, 50 psi; ion source gas 2, 50 psi; declustering potential, −40 V; and collision energy, −20 V. The following MRMs were used: methylcitrate, 204.9/125.0; Hydroxypropionate 88.9/59.0; malate 133/115; methylmalonate 117/73; alpha ketoglutarate 145/101; warfarin 307.1/161.0, and creatinine 114.1/44. The system was controlled by the Analyst® software (Sciex) and analyzed with MultiQuant™ 3.0.2 software (Sciex). The relative amount of metabolite was normalized to the amount of warfarin then the ratio to creatinine was calculated.

Data analysis: Statistical significance (p values) was determined using a two-tailed Student's t test using GraphPad software.

### Results and Discussion

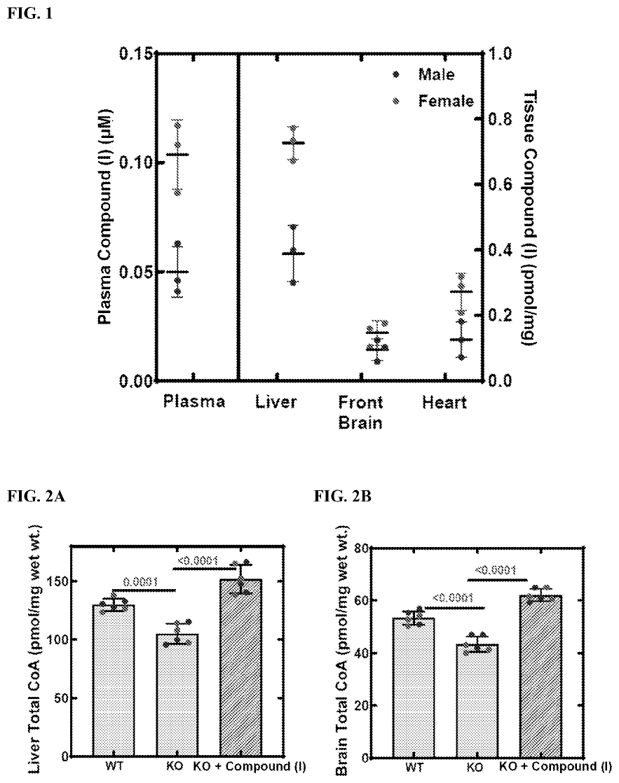

Plasma and Tissue Concentrations of Compound (I) in Pcca^{-/-}PCCA(A138T)^{tg/0} mice: Plasma and tissue (liver, brain, and heart) concentrations of Compound (T) in Pcca^{-/-}PCCA(A138T)^{tg/0} mice after 22.5 ppm (3 mg/kg) of Compound (I) in chow for 30 days after weaning, are presented in FIG. 1. Compound (I) appeared to be well distributed into the liver, and to a lesser extent in brain and heart, and in general, female mice appeared to have higher exposures than males.

Total CoA in Liver and Brain of Pcca^{-/-}PCCA(A138T)^{tg/0} mice with and without Compound (I): The ability of Compound (I) to increase liver and brain CoA levels was also tested in Pcca^{-/-}PCCA(A138T)^{tg/0} mice. Pcca^{-/-}PCCA(A138 T)^{tg/0} mice exhibit significantly decreased total CoA in liver and brain tissue (˜19% lower than wild-type). Treatment with Compound (I) increased both liver and brain CoA levels in Pcca^{-/-}PCCA(A138T)^{tg/0} mice by ˜45% and ˜44%, respectively, beyond wildtype levels, compared to untreated Pcca^{-/-}PCCA(A138T)^{tg/0} mice (FIGS. 2A-2B).

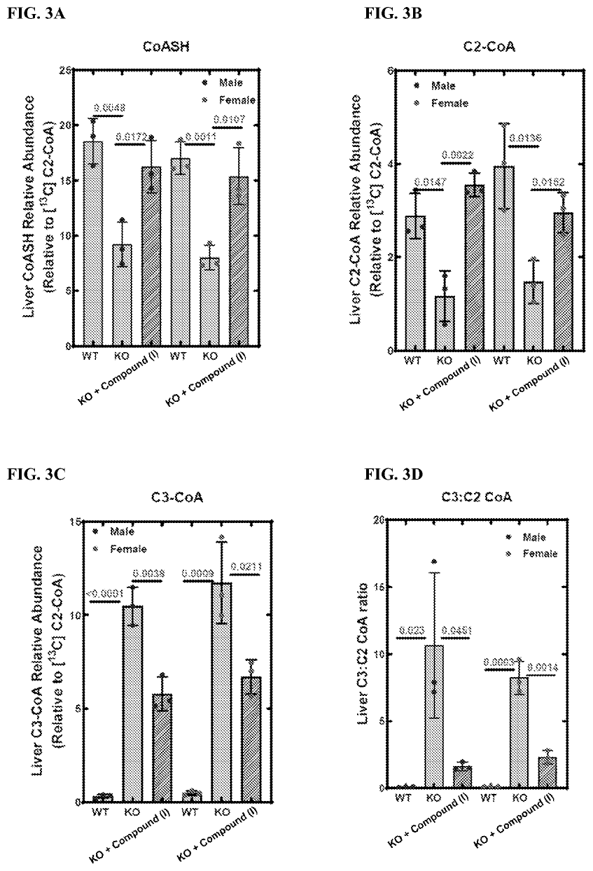

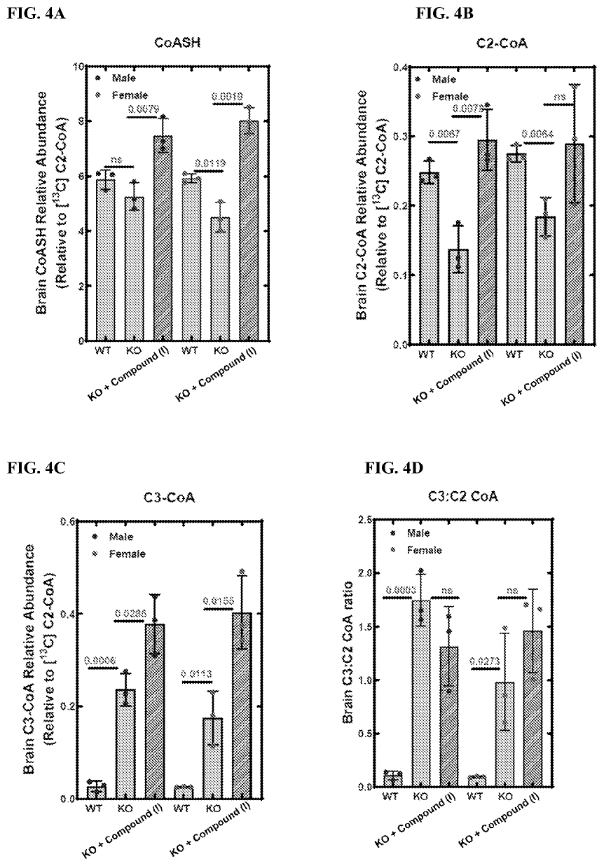

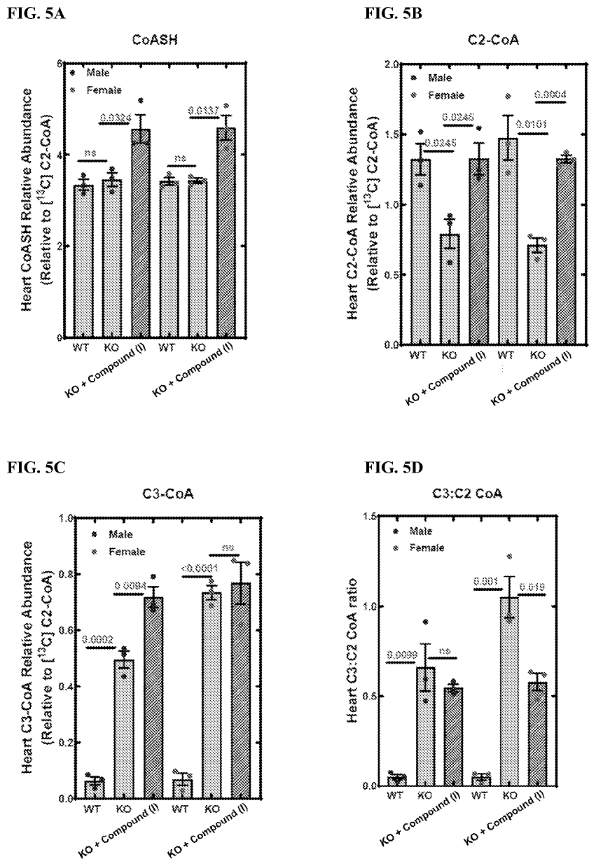

Acyl-CoA in Liver, Brain, and Heart from Wild-Type and Pcca^{-/-}PCCA(A138T)^{tg/0} mice with and without Compound (I): Free CoA (CoASH) and acetyl-CoA (C2-CoA) are the most abundant components of the total cellular CoA pool in the non-disease state and are central to the operation of intermediary metabolism. Accumulation of C3-CoA in PA disease draws on the CoASH pool and diminishes the availability of CoASH primarily for formation of C2-CoA in addition to many other processes. Examination of acyl CoAs in liver, brain, and heart of Pcca^{-/-}PCCA(A138T)^{tg/0} mice showed significantly decreased C2-CoA in diseased animal tissue, as expected, vs. wild-type, which was significantly improved with Compound (I) and rescued to wild-type levels. The C3:C2 CoA ratio was significantly increased in Pcca^{-/-}PCCA(A138T)'s mice and Compound (I) improved the C₃:C2 CoA ratio in liver and heart tissue (FIGS. 3A-3D, 4A-4D, and 5A-5D).

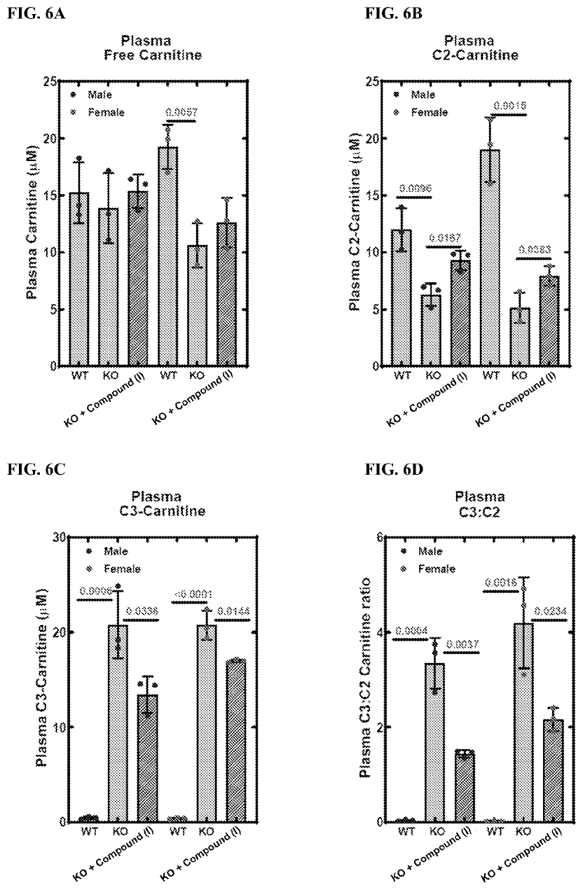

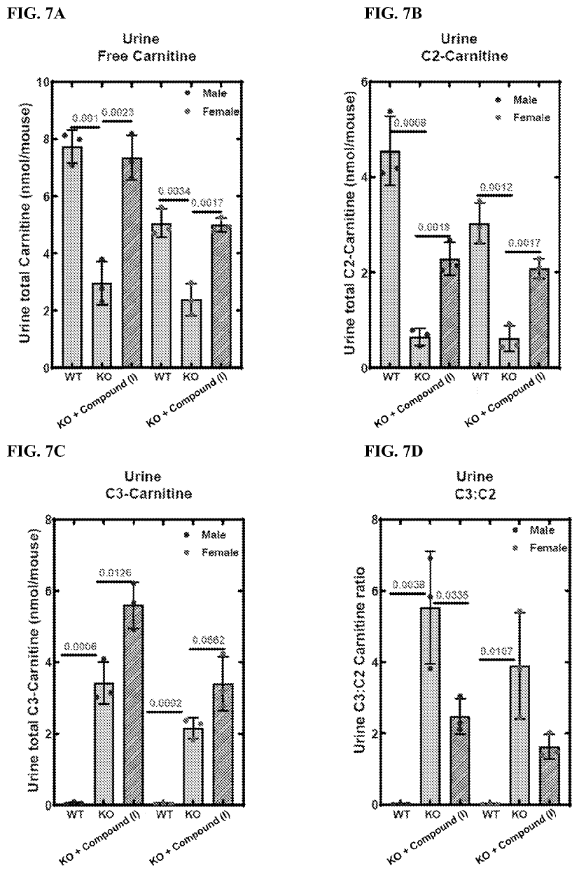

Plasma and Urine Acyl Carnitines in Wild-Type and Pcca^{-/-}PCCA(A138T)^{tg/0} mice with and without Compound (I): Plasma acylcarnitine profiles are often utilized in patients to confirm the diagnosis of an organic acidemia. In particular, in the acylcamitine profile, elevated C3-carnitine is a hallmark of PA and MMA. Significant improvements in PA-associated plasma (FIGS. 6A-6D) and urine (FIGS. 7A-7D) biomarkers were observed with Compound (I) treatment of Pcca^{-/-}PCCA(A138T)^{tg/0} mice. Compound (I) treatment rescued decreased free carnitine and C2-carnitine levels in Pcca^{-/-}PCCA(A138T)^{tg/0} mice plasma and urine. Interestingly, Compound (I) decreased C3-camitine in plasma but increased C3-carnitine in urine suggesting that more C3-carnitine is being excreted into the urine with treatment. Importantly, Compound (I) significantly reduced the elevated C3:C2 ratio observed in Pcca^{-/-}PCCA(A138T)^{tg/0} mice in both plasma and urine.

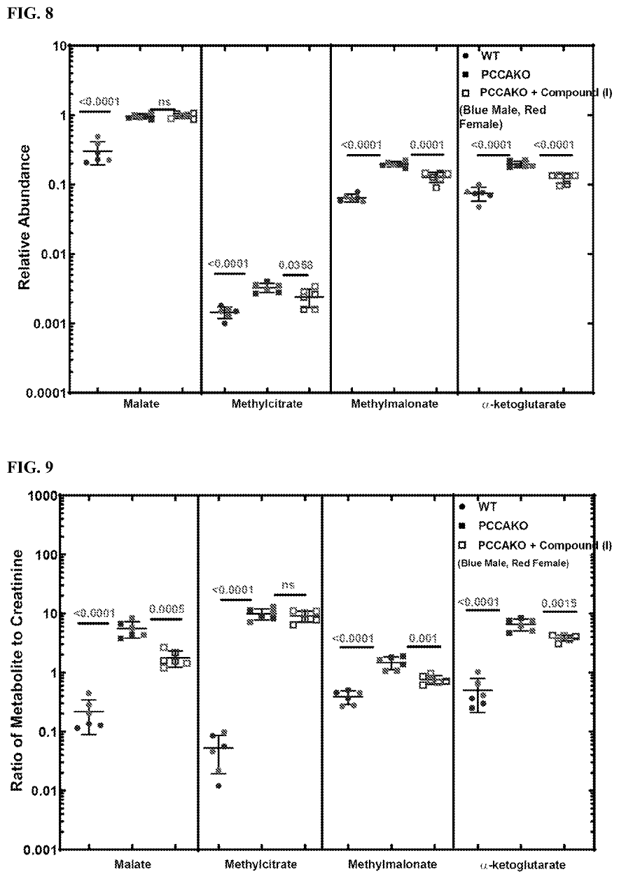

Plasma and Urine Metabolites in Wild-Type and Pcca^{-/-}PCCA(A138T)^{tg/0} mice with and without Compound (I): In Pcca^{-/-}PCCA(A138T)^{tg/0} mice, the TCA cycle is impaired, leading to leaking of CoA-dependent TCA-intermediates into plasma and urine, as evidenced by significant increases in malate, methylcitrate, methylmalonate, and α-ketoglutarate in plasma and urine from Pcca^{-/-}PCCA(A138T)^{tg/0} mice (FIG. 8 and FIG. 9). Compound (I) treatment reduced levels methylcitrate, methylmalonate, and α-ketoglutarate in plasma, and malate, methylmalonate and α-ketoglutarate in urine suggesting restoration of TCA cycle function in Pcca^{-/-}PCCA(A138T)^{tg/0} mice.

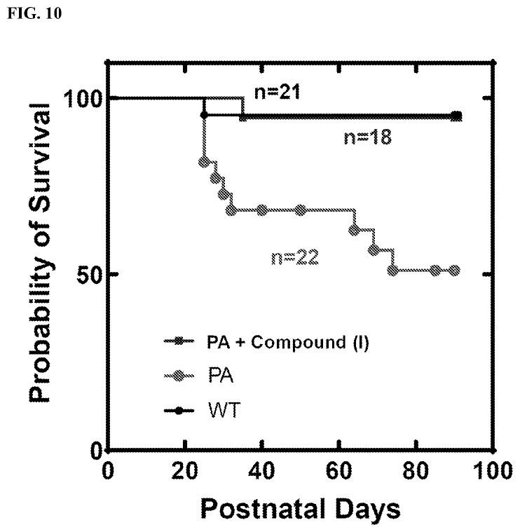

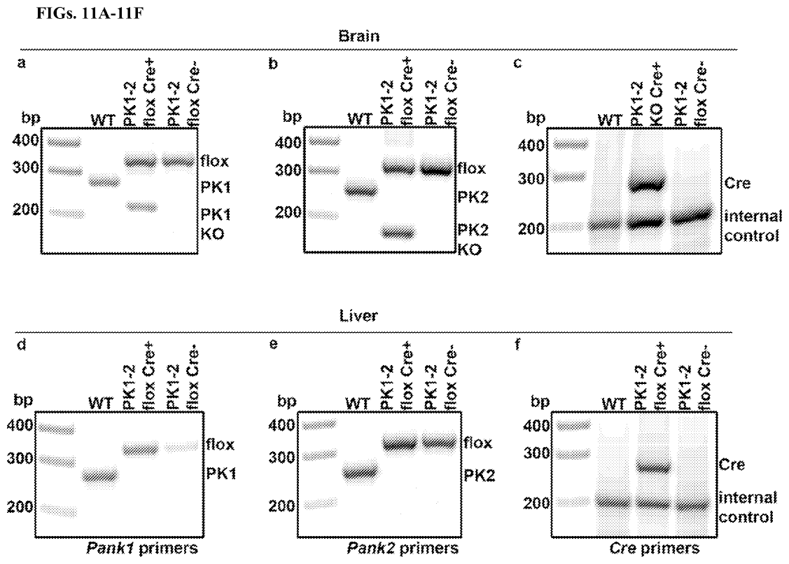

Survival in Wild-Type and Pcca^{-/-}PCC(A138T)^{tg/0} mice with and without Compound (I): While survival of Pcca^{-/-}PCCA(A138T)^{tg/0} mice is distinctly longer than Pcca^{-/-} mice, which only survive for 36 hours post-birth, Pcca^{-/-}PCCA(A138T)^{tg/0} mice have decreased survival in the 3 months following birth. At postnatal day 90, the probability of survival of Pcca^{-/-}PCCA(A138T)^{tg/0} mice was ˜51%. Treatment of Pcca^{-/-}PCCA(A138T)^{tg/0} mice with Compound (I) significantly improved the probability of survival to ˜94% at postnatal day 90 (FIG. 10).

Conclusions: Compound (1) treatment increased liver and brain CoA levels in Pcca^{-/-}PCCA(A138T)^{tg/0} mice, a mouse model of PA. Importantly, treatment with Compound (I) improved multiple hallmarks of PA in Pcca^{-/-}PCCA(A138 T)^{tg/0} mice including liver, heart, and brain acyl-CoAs, plasma and urinary acyl-carnitines, levels of TCA cycle intermediates in plasma and urine, and probability of survival compared to untreated animals.

### Example 2: Efficacy of Compound (I) in SynCre⁺Pank1^{-/-}Pank2^{-/-} Mice

Compound (I) has been demonstrated to selectively and potently bind to both PanK2 and PanK3 with Kᵢs of less than 5 nM. As PKAN results from mutations in the Pank2 gene, the activity of PanK3 in the central nervous system (CNS) of PKAN animals is of interest.

In initial versions of an animal model for PKAN, the Pank2 gene was inactivated in mice. However, this model was considered too mild to be a relevant animal model of PKAN as the animals had normal movement and lifespan and exhibited retinal degeneration and azoospermia as the only overt phenotypes. Brain iron accumulation was not detectable by magnetic resonance imaging in this model. Brain CoA deficiency was also not observed in adult animals, but a transient brain CoA deficiency was identified during the postnatal growth period of Pank2^{-/-} mice. Wild-type mice maintained on a pantothenate-deficient diet for 8 months eventually developed a movement disorder and azoospermia, linking these two phenotypes to CoA deficiency. Later, examination of selected brain regions of Pank2^{-/-} adult animals found evidence for perturbation of gene expression related to iron and dopamine metabolism, as well as altered mitochondrial functional capacity, and higher iron levels in the globus pallidus of the brain using more sensitive mass spectrometry technology. Thus, the Pank2^{-/-} mouse model represents mild PKAN disease. As a result, investigation of therapies for PKAN in this model is challenging due to the mild biochemical and limited pathological phenotypes of disease in these animals.

SynCre⁺Pank1^{-/-}Pank2^{-/-} Mouse Model

To develop a mouse model with a more severe PKAN phenotype to use as a platform to test therapies for the disease, a distinct, conditional mouse model with deletions of both Pank1 and Pank2 genes in neurons was developed to obtain a durable CoA deficiency in the brain. The SynCre⁺Pank1^{-/-}Pank2^{-/-} neuronal knockout mouse model established a connection between brain CoA deficiency and reduced growth rate, reduced lifespan and movement dysfunction. Brain iron accumulation is not observed in this model. Although the Pank genetic deficiencies were limited to neurons and did not occur in all murine cell types, neurons have been identified as the most affected cell types in post-mortem brains of PKAN patients. The neuron-specific, constitutive SynCre⁺Pank1^{-/-}Pank2^{-/-} knockout mouse model provides tractable phenotypic readouts, including reduced lifespan, weight loss, and significant locomotor defects, that resemble aspects of PKAN for development of an efficient therapeutic to replenish a substantial CoA deficiency in brain.

Derivation of SynCre⁺Pank1^{-/-}Pank2^{-/-} mice was described previously by Sharma et al. (Sharma, L. K., et al., “A therapeutic approach to pantothenate kinase associated neurodegeneration,” Nat. Commun. 9, 4399, (2018)). Pank1^{fl/fl} and Pank2^{fl/fl} mice were generated as reported (Leonardi, R., et al., Pantothenate kinase 1 is required to support the metabolic transition from the fed to the fasted state. PLoS ONE. 5, e11107 (2010); Garcia, M., et al., Germline deletion of pantothenate kinases 1 and 2 reveals the key roles for CoA in postnatal metabolism. PLoS One. 7, e40871 (2012)). The SynCre transgene originated in B6·Cg-Tg(Syn1-cre)671Jxm/J transgenic mice (The Jackson Laboratory) that express the Cre recombinase driven by the synapsin1 (Syn) promoter. SynCre⁺Pank1^{fl/fl}Pank2^{fl/+} (or SynCre⁺Pank1^{fl/+}Pank2^{fl/fl}) females were mated with SynCre⁰Pank1^{fl/fl}Pank2^{fl/fl} males to obtain SynCre⁺Pank1^{fl/fl}Pank2^{fl/fl} progeny that had both Pank1 and Pank2 conditionally deleted in neurons. Control mice obtained from littermates had the SynCre⁰Pank^{fl/fl}Pank2^{fl/fl} genotype. PCR genotyping primer pairs and products are listed in Table 2 (Supplementary Table 4 from Sharma et al.). Primer sets used to detect the presence or absence of the SynCre transgene, and to detect the presence of the floxed and knockout alleles of the Pank1 and Pank2 genes. PCR analysis was used to genotype tail biopsies where a 338 bp product indicated the floxed Pank1 allele, a 332 bp product indicated the floxed Pank2 allele, and a 285 bp product indicated the presence of the Cre transgene. The progeny followed a Mendelian inheritance pattern and 1 in 4 pups were the desired genotype SynCre⁺Pank1^{-/-}Pank2^{-/-}. Wild-type matched control animals were derived from breeding pairs that were heterozygous for both Pank1-floxed and Pank2-floxed alleles and lacked the SynCre transgene. To confirm the neuronal enrichment of the floxed gene deletions, PCR genotyping was performed for the brain and liver of the animals (FIGS. 11A-11F). Neuron-specific deletion of the Pank1- and Pank2-floxed alleles was confirmed in brains by the presence of a 218 base pair (bp) and 176 bp product, respectively, that were absent in liver. Incomplete deletion of the Pank1- and Pank2-floxed alleles in brain was indicated by coincidence of the Pank1-floxed and Pank1-deleted PCR products, and the Pank2-floxed and Pank2-deleted PCR products. Cell types other than neurons contributed to the undeleted, residual Pank1- and Pank2-floxed genes in brain. Following genotyping, SynCre⁺ and SynCre⁰Pank1-floxed and Pank2-floxed mice were randomly enrolled into the treatment or control arms of the Compound (I) trial as they emerged from the breeding program. Mouse colony database management was performed using StudyLog software.

TABLE 2
Genotyping primers to identify SynCre-
Pank1, Pank2 mice

| | Primer | Sequence | PCR |
|---|---|---|---|
| Gene | Name | 5′-3′ | conditions |
| Pank1 | Pankl | GGATAGGA | 94º C., 5 min + |
| | F1 | TGGCTACT | 40 cycles |
| | | AGCTGC | (94° C., 30 sec + |
| | | (SEQ ID | 58° C., 30 sec + |
| | | NO.: 12) | 72º C., 30 sec) + |
| | Pankl | TTACTAGC | 72° C., 5 min + |
| | R1 | TAAGTGGC | 4° C., ∞ |
| | | CCAGG | Pank1 F1 + |
| | | (SEQ ID | Pankl R1 + |
| | | NO.: 13) | Pank1 R2 |
| | Pankl | GCCTAATT | WT = 274 bp, |
| | R2 | TCGTTCAC | Floxed = 338 bp, |
| | | AGTGG | KO = 218 bp |
| | | (SEQ ID | |
| | | NO.: 14) | |
| Pank2 | Pank2 | CTGGAAAT | 94º C., 5 min + |
| | F1 | CTCGTGTA | 40 cycles |
| | | GTTGAGTC | (94° C., 30 sec + |
| | | (SEQ ID | 58° C., 30 sec + |
| | | NO.: 15) | 72° C., 1 min) + |
| | Pank2 | TCAAGCAG | 72° C., 5 min + |
| | R1 | TATCAAAG | 4° C., ∞ |
| | | ACACCAC | Pank2 F1 + |
| | | (SEQ ID | PanK2 R1 + |
| | | NO.: 16) | PanK2 R2 |
| | Pank2 | CACTGGAG | WT = 262 bp, |
| | R2 | CAGTAACT | Floxed = 332 bp, |
| | | GAGAGC | KO = 176 bp |
| | | (SEQ ID | |
| | | NO.: 17) | |
| Cre | 15563 | CTCAGCGC | 94º C., 5 min + |
| | (Cre) | TGCCTCAG | 45 cycles |
| | | TCT | (94° C., 30 sec + |
| | | (SEQ ID | 60° C., 30 sec + |
| | | NO.: 18) | 72° C., 45 sec) + |
| | 15564 | GCATCGAC | 72° C., 5 min + |
| | (Cre) | CGGTAATG | 4° C., ∞ |
| | | CA | Cre = 285 bp, |
| | | (SEQ ID | Internal |
| | | NO.: 19) | Control = 200 bp |
| | IMR0015 | CAAATGTT | |
| | (internal | GCTTGTCT | |
| | control) | GGTG | |
| | | (SEQ ID | |
| | | NO.: 20) | |
| | IMR0016 | GTCAGTCG | |
| | (internal | AGTGCACA | |
| | control) | GTTT | |
| | | (SEQ ID | |
| | | NO.: 21) | |

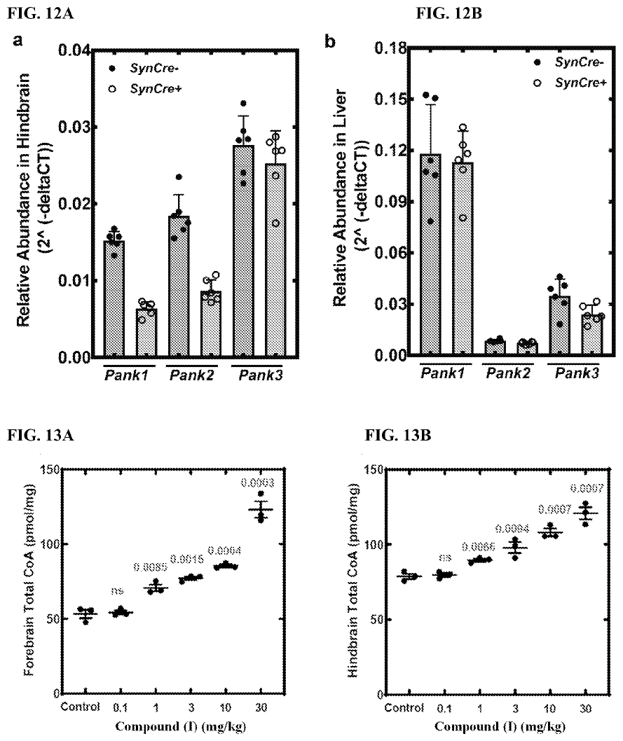

The abundance of the three Pank mRNAs in mouse liver and brain was determined by quantitative real-time PCR using primer pairs listed in Table 3 (Supplementary Table 5 from Sharma et al.). The primers for mouse glyceraldehyde-3-phosphate dehydrogenase (Gapdh) used as the calibrator were purchased from Applied Biosystems (catalog #4308313). RNA was isolated from cryopreserved liver or brain tissue. Synthesis of first-strand cDNA was obtained by reverse transcription using SuperScript™ RNase H reverse transcriptase, the RNA templates and random primers. Quantitative real-time PCR was performed in triplicate using the ABI Prism®7700 Sequence Detection System. All of the values were compared using the C_{T} method (Winer et al., 1999), and the amount of cDNA (2^{-ΔcT}) was calculated relative to Gapdh mRNA. Endogenous Pank3 expression was confirmed to be at normal levels in brain in the SynCre⁺Pank1^{-/-}Pank2^{-/-} mice, Pank1 expression was reduced >60%, and Pank2 expression was reduced >50% (FIGS. 12A-12B). In contrast, the liver expression levels of the Pank1, Pank2, and Pank3 genes was normal in the SynCre⁺Pank1^{-/-}Pank2^{-/-} animals. Cell types other than neurons contributed to the remaining, low Pank1 and Pank2 gene expression levels in the SynCre⁺Pank1^{-/-}Pank2^{-/-} brain.

TABLE 3
RT-qPCR primers for mouse PanKs

| | Forward | Reverse |
|---|---|---|
| | Primer | Primer |
| Gene | (5′-3′) | (5′-3′) |
| Pank1 | TCAGGGCC | TGGAGTAC |
| Exon 4 | TTCTTTAC | ACTGCCAG |
| | GTTG | GATG |
| | (SEQ ID | (SEQ ID |
| | NO.: 22) | NO.: 23) |
| Pank2 | CCCTGCTG | GAGCCGAT |
| Exon 3 | ATTCTGAA | GTTCACCA |
| | AAATG | GAAG |
| | (SEQ ID | (SEQ ID |
| | NO.: 24) | NO.: 25) |
| Pank3 | AACCTCCA | TGGGTAAG |
| Exon3 | CCTGCACA | GATCATCC |
| | AACT | AGGT |
| | (SEQ ID | (SEQ ID |
| | NO.: 26) | NO.: 27) |

SynCre⁺Pank1^{-/-}Pank2^{-/-} Mouse Experiments

The mice were maintained at 72°±2° F., humidity 50%±10% and a 14-hour light/10-hour dark cycle with the dark cycle starting at 20:00 hrs. Water was supplied ad libitum. Mice were maintained on a chow diet with purified ingredients containing 19% kcal protein, 66.5% kcal carbohydrate and 14.4% kcal fat (Envigo #TD170542) which was similar to standard rodent chow (LabDiet #5013). The mice were randomized into the treatment arms. Compound (I) was formulated at 22.5 ppm (3 mg/kg/day) (Envigo #TD190846) or 75 ppm (10 mg/kg/day)(Envigo #TD200028) in chow and SynCre⁺Pank1^{-/-}Pank2^{-/-} animals were maintained on this diet ±Compound (I) for 25 days after weaning at postnatal day (P) 20. Assuming a wild-type adult mouse cats 4 g chow/day and weighs 30 g, 22.5 ppm in chow is approximately equivalent to 3 mg/kg/day in chow, and 75 ppm is approximately equivalent to 10 mg/kg/day. However, this is an approximation, as SynCre⁺Pank1^{-/-}Pank2^{-/-} mice both weigh less and eat less than wild-type mice. At P45, the spontaneous activity of each animal in a novel open field provided a general measure of motor function of the SynCre (+/−) Pank1, Pank2-floxed mice, treated or not with Compound (I) in chow. At the end of the dark cycle, individual mice were placed in an open rectangular arena (36.8 cm×43.2 cm) for 5 min during the light cycle and motor activity was evaluated using a video tracking system provided by HVS Image with associated 2100 Plus software (San Diego, CA, USA). Each animal was placed in the center of the arena under standard overhead lighting and the total distance traveled and the percentage of time in motion were recorded. The behaviors of age-matched control animals, treated with Compound (I) and untreated, were quantified and compared. For biochemical analyses, at four hours after the end of the dark cycle, blood was collected in EDTA at P45 by cardiac puncture from isoflurane-anesthetized animals, plasma was prepared and stored frozen at −80° C. until analysis. Liver, forebrain and hindbrain were quickly excised, approximately 30-50 mg pieces were immediately flash frozen in liquid N₂ and stored frozen at −80° C. until analysis. The plasma and tissue samples were used for Compound (I) and total CoA determinations.

The relative abundances of neurochemical metabolites using magnetic resonance spectroscopy (MRS) at approximate age P45 were determined in separate cohorts of the 3 groups of SynCre[^{+/0}]Pank1, Pank2-floxed mice, treated or not with Compound (I) in chow. The ¹H MRS studies were performed on a Bruker Clinscan 7T magnetic resonance imaging (MRI) scanner (Bruker BioSpin MRI GmbH, Ettlingen, Germany). Mice were anesthetized using isoflurane mixed with oxygen (1-2%) and the respiration rate was monitored. Each MRI was acquired with a mouse brain surface receive coil positioned over the mouse head and placed inside a 72 mm transmit/receive coil. After the localizer, a T₂-weighted turbo spin echo sequence was performed in the coronal (TR/TE=2290/41 ms, matrix size=192×256, slice thickness 0.5 mm, number of slices=14) and axial (TR/TE=3841/50 ms, matrix size=192×144, slice thickness 0.4 mm, number of slices=42) orientations. The T₂-weighted scans were used to position a 3.5×4.5×2.0 mm³ voxel for spectroscopy in the midbrain to cover thalamus and hippocampus. A ¹H MR spectrum was generated with that voxel using a PRESS sequence (repetition time/echo time=3000/11 ms, averages=128, data length=2048, spectral width=2900 Hz). The in vivo ¹H MRS data were quantified as the Metabolite to tCr ratio using LCModel software (v.6.3), a widely applied MRS analysis tool that employs a least-squares-based prior-knowledge fitting program. The concentration of metabolites was measured using the unsuppressed water peak as a concentration standard. LCModel applied a 7T spin echo (TE=11 ms) basis set incorporating the following resonances: alanine (Ala), aspartic acid (Asp), creatine (Cre), phosphocreatine, γ-amino butyric acid (GABA), glucose, [glutamine plus glutamic acid](Glx), glycerophosphocholine, phosphocholine, glutathione, myo-inositol (mIns), N-acetyl aspartate (NAA), NAA+Glu, sycllo-inositol and taurine, with lipid resonances at 0.9, 1.3 and 2.0 ppm and macromolecule resonances at 0.9, 1.2, 1.4, 1.7, and 2.0 ppm.

Compound (I) Extraction and Quantification by LC/MS/MS: Plasma (20 μL) was added to 100 μL acetonitrile containing 0.6 μM warfarin to a final concentration of 0.5 μM. The samples were incubated on ice for 30 min. Samples were spun at 3500×g for 10 min to pellet debris, and the supernatant was transferred to a glass vial. A Compound (I) standard curve was created by spiking in known concentrations of Compound (I) into 20 μL of plasma from a control mouse and following the above procedure. 30 mg of tissue was homogenized in 2 mL of 80% methanol containing 0.1 μM warfarin and incubated at −80° C. for 4 hr. Samples were spun at 3500×g for 10 min to pellet debris, supernatant was transferred to a glass tube and dried down using a Savant SPD1010 Speed-Vac (Thermo Scientific) overnight. Samples were resuspended in 400 μL of 80% acetonitrile to a final concentration of 0.5 μM and transferred to a glass vial.

Compound (I) was analyzed using a Shimadzu Prominence UFLC attached to a QTrap 4500 equipped with a Turbo V ion source (Sciex). Samples (5 μL) were injected onto an XSelect® HSS C18, 2.5 μm, 3.0×150 mm column (Waters) using a flow rate of 0.25 mL/min. Solvent A was 0.1% formic acid in water, and Solvent B was acetonitrile with 0.10% formic acid. The HPLC program was the following: starting solvent mixture of 50% B, 0 to 0.5 min isocratic with 50% B; 0.5 to 1.5 min linear gradient to 95% B; 1.5 to 20 min isocratic with 95% B; 20 to 21 min linear gradient to 50% B; 21 to 25 min isocratic with 50% B. The QTrap 4500 was operated in the positive mode, and the ion source parameters were: ion spray voltage, 5500 V; curtain gas, 30 psi; temperature, 450° C.; collision gas, medium; ion source gas 1, 30 psi; and ion source gas 2, 40 psi. The MRM transition for Compound (I) was 375.2/199.1 m/z and for warfarin was 309.1/163.0 m/z both with a declustering potential, 65 V and collision energy, 30 V. The system was controlled by the Analyst® software (Sciex) and analyzed with MultiQuant™ 3.0.2 software (Sciex).

Tissue Total CoA Determinations: Total CoA was quantified as described earlier. Either frozen brain or liver tissue (30-40 mg) was homogenized in 1 mM potassium hydroxide (KOH) to hydrolyze all the acyl chains and then the CoA was derivatized with monobromobimane (mBBr). The CoA bimane product was purified on a SPE 2-(2-pyridyl) ethyl column and analyzed by HPLC with fluorescent detection λₑₓ=393 nm, λₑₘ=470 nm. The retention time was determined by running a CoA standard before each set of samples. Total CoA was quantified using a standard curve generated with known amounts of CoA.

Data analysis: Statistical significance (p values) was determined using a two-tailed Student's t test using GraphPad software. The MRS parameters were analyzed using ‘R’ 4.0.2 and the mean values and standard deviations were calculated for each group of mice. Wilcoxon Rank sum tests were used to test whether MRS parameters were different between SynCre⁺Pank1^{-/-}Pank2^{-/-} mice untreated or treated with Compound (I), or control SynCre⁰Pank1^{fl/fl}Pank2^{fl/fl} wild-type mice. A p value <0.05 was considered statistically significant.

### Results and Discussion

In vitro studies: Compound (I) (10 M) stabilized PanK3 against thermal denaturation as measured by differential scanning fluorometry. Compound (I) counteracted the feedback loop of inhibition of PanK enzymes by acetyl-CoA (Table 4).

TABLE 4
Half maximal concentration of Compound (I) required for
alleviation of acetyl-CoA inhibiton of PanK activity

| | Average IC₅₀ (μM) ± SD | | |
|---|---|---|---|
| | Without | With | Compound (I) |
| PanK | Compound (I) | Compound (I) | Concentration (μM) |
| PanK1β | 2.58 ± 0.23 | >100 | 7.5 |
| PanK2 | 0.44 ± 0.06 | 7.64 ± 4.74 | 3 |
| PanK3 | 1.44 ± 0.09 | >100 | 2 |

SD = standard deviation;
IC₅₀ = half maximal inhibitory concentration

Effect of Compound (I) therapy on CoA levels in forebrain and hindbrain of SynCre⁺Pank1^{-/-}Pank2^{-/-} mice: Compound (I) treatment raises intracellular CoA concentrations in cells and in tissues. The response of forebrain and hindbrain CoA levels was determined following oral administration of Compound (I) to wild-type mice, demonstrating a dose-responsive increase in brain CoA with increasing concentrations of Compound (I), as shown in FIGS. 13A-13B. C57BL/6J (wild-type) mice were administered Compound (I) by oral gavage once daily (QD) at 0.1, 1, 3, 10, or 30 mg/kg in 0.5% methylcellulose for one week. Control animals were administered 0.5% methylcellulose QD for one week. Four hours after the last dose, the animals were sacrificed, plasma and tissues were collected, and CoA levels were determined in tissues. Total CoA levels in forebrain (FIG. 13A) and hindbrain (FIG. 13B) are shown. Statistical significance was determined for total CoA using a Student's t-test calculated with GraphPad software and P-values compared to the control animals are noted on the figure panels.

In Syn-Pank1^{-/-}Syn-Pank2^{-/-} neuronal knockout (Pank1,2 neural knockout) brains, CoA levels are significantly reduced by −26% compared to wild-type matched control animals and locomotion of Pank1,2 neural knockout mice is significantly impaired compared to controls. Treatment of Pank1,2 neural knockout mice with Compound (I) significantly increased forebrain CoA levels by ˜70% vs untreated mice, as shown in FIG. 14. Wild-type (WT) mice and Pank1,2 neural knockout mice (PK1,2 dKO) were maintained on chow with and without Compound (I) (75 ppm) starting at postnatal day 14 and CoA levels were assessed at postnatal day 45. Statistical significance was determined using a two-tailed Student's t test (GraphPad software) and P-values are shown about the bars, N=6-8. Black circles show individual animal data and bars depict means t standard error of the mean (SEM).

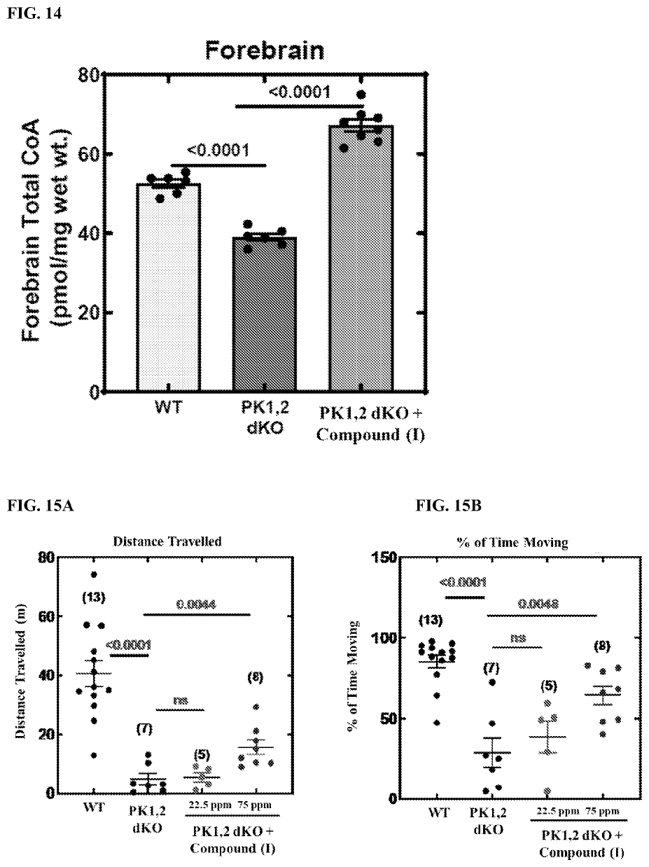

Effect of Compound (I) therapy on movement and distance travelled in SynCre⁺Pank1^{-/-}Pank2^{-/-} mice: Compound (I) treatment was initiated at postnatal day 14, after disease was established, and data from locomotion experiments were collected at postnatal day 45. Treatment of Pank1,2 neural knockout mice with 22.5 ppm Compound (I) showed some improvement in movement. Treatment with 75 ppm Compound (I) significantly increased locomotor activity as demonstrated by an increase in the percent of time moving (˜2.2-fold increase) and the distance travelled while moving (˜3-fold increase) relative to untreated mice (FIGS. 15A-15B). Wild-type mice were compared to Pank1,2 neural knockout mice (PK1,2 dKO) at postnatal day 45 with or without Compound (I) (22.5 ppm or 75 ppm in chow) starting at postnatal day 14. FIG. 15A shows distance travelled by mice in the 5 min open field test; and FIG. 15B shows percent of time mice were moving during the 5 min open field test. Data are means±standard error of the mean (SEM) and statistical significance was determined using a two-tailed Student's t test (GraphPad software). P-values are shown in red.

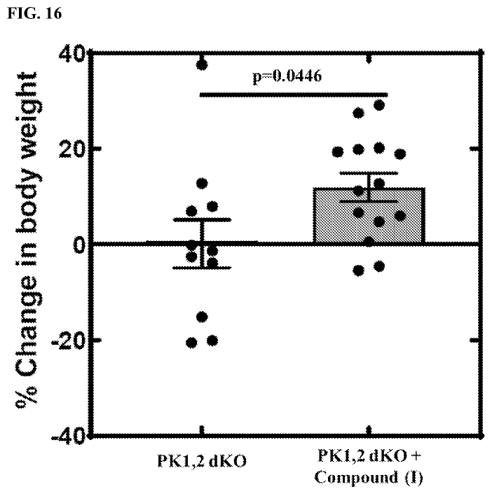

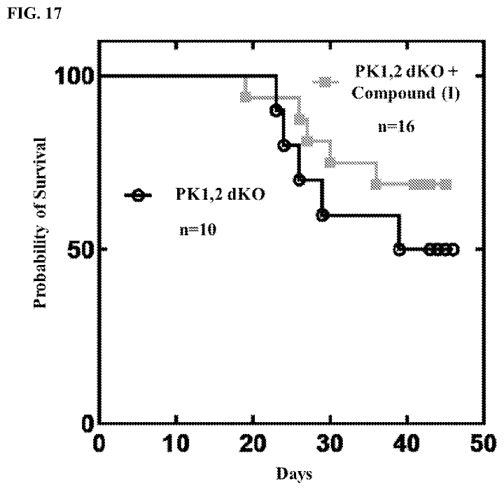

Body Weight and Lifespan in SynCre⁺Pank1^{-/-}Pank2^{-/-} mice with and without Compound (I): Pank1,2 neural knockout mice (PK1,2 dKO) develop normally until postnatal day 12.5, then begin to lose weight and have a median survival of 52 days. Given Compound (T) treatment is initiated after disease is established, when phenotypic changes have already been observed, including smaller size and reduced movement, these studies represent investigation of Compound (I) in a treatment model of PKAN. Pank1,2 neural knockout mice treated with Compound I) had significantly increased body weight gain compared with untreated mice, as shown in FIG. 16. Additionally, Pank1,2 neural knockout mice treated with Compound (I) had a greater probability of survival compared with untreated mice until postnatal day 45 (FIG. 17). Probability of survival at postnatal Day 45 was −70% for animals treated with Compound (I) vs. ˜50% for untreated animals. Following analysis of movement, all animals were sacrificed at postnatal Day 45 for examination of tissue CoA levels. Compound (I) treated mice did not reach median survival lifespan as the animals were harvested for experimental determination at postnatal day 45.

Neurometabolites in SynCre⁺Pank1^{-/-}Pank2^{-/-} mice with and without Compound (I): The ¹H MRS spectra acquired from the midbrain of the mice at P45 showed significant reduction in the Glx, NAA, and lactate signals at 2.35, 2.0 and 1.33 ppm, respectively. Creatine levels were unchanged among the 3 groups (control SynCre⁺Pank1^{fl/fl}Pank2^{fl/fl}, SynCre⁺Pank1^{-/-}Pank2^{-/-} untreated, and SynCre⁺Pank1^{-/-}Pank2^{-/-} mice treated with Compound (I)). The unsuppressed water signal was not acquired and so total creatine levels (tCr) were used to normalize the relative amounts of the affected neurometabolites (Table 5). Treatment with Compound (I) (10 mg/kg/day) restored the Glx/tCr, whereas NAA/tCr and Lac/tCr were partially restored in the SynCre⁺Pank1^{-/-}Pank2^{-/-} mice following treatment. Glx is the summation of glutamic acid, the most abundant excitatory neurotransmitter in brain, and glutamine, the main precursor for glutamic acid. NAA is the most abundant neurometabolite and an indicator of neuronal integrity and function. Lac levels represent the net balance between lactate production from cerebral glycolysis and lactate oxidative consumption to maintain the neuronal redox state.

TABLE 5
Neurometabolites in SynCre⁺ Pank1^{-/-} Pank2^{-/-} mice with and without Compound (I)

| | SynCre⁰ Pank1^{fl/fl} Pank2^{fl/fl} | | SynCre⁺ Pank1^{-/-} Pank2^{-/-} |
|---|---|---|---|
| | Control | SynCre⁺ Pank1^{-/-} Pank2^{-/-} | With Compound (I) |
| Glx | 1.60 ± 0.21 | 1.32 ± 0.17** | 1.62 ± 0.11 |
| GABA | 0.37 ± 0.04 | 0.33 ± 0.03* | 0.40 ± 0.02 |
| NAA | 0.72 ± 0.04 | 0.64 ± 0.03** | 0.67 ± 0.04* |
| Lac | 0.25 ± 0.08 | 0.11 ± 0.05* | 0.20 ± 0.07 |

*p < 0.05;
**p < 0.01
Glx/tCr p = 0.002, WT vs Pank1, 2 KO**
p = 0.30, WT vs Pank1, 2 KO + Compound (1)
GABA/tCr
p = 0.04, WT vs Pank1, 2 KO*
p = 0.21, WT vs Pank1, 2 KO + Compound (I)
NAA
p = 0.002, WT vs Pank1, 2 KO**
p = 0.022, WT vs Pank1, 2 KO + Compound (1) *
Lac
p = 0.04, WT vs Pank1, 2 KO + Compound (1) *
p = 0.22, WT vs Pank1, 2 KO + Compound (1)

Overall, Compound (I) treatment increased brain CoA levels in SynCre⁺Pank1^{-/-}Pank2^{-/-} mice with neuronal CoA deficiency that models the brain pathology in PKAN. Importantly, treatment improved severe movement dysfunction, a hallmark of PKAN, improved growth rate and extended survival in SynCre⁺Pank1^{-/-}Pank2^{-/-} mice following treatment with 75 ppm (10 mg/kg/day) that corresponded with higher plasma concentration and greater brain exposure to Compound (T). Treatment with Compound (T) also restored levels of the neurometabolites Glx and GABA, indicating improvement of neurotransmitter metabolism, and Lac, suggesting improvement of brain glycolytic metabolism. Partial restoration of NAA with Compound (I) treatment indicated improvement of neuronal integrity and/or function.

Caveats to these data include that measurement of tissue CoA levels in SynCre⁺Pank^{-/-}Pank2^{-/-} mice reflect a single snapshot in time and do not provide any information on the duration of effect of Compound (I) on tissue CoA levels. Studies of the effects of Compound (I) treatment in male C57BL/6J mice (CoA-PC-004) on CoA levels in the brain and liver over a period of 24 hours show a sustained duration of PD effect. Lastly, human Compound (I) exposure via an oral tablet or suspension dosing regimen will be difficult to correlate to exposures achieved with 22.5 ppm (˜3 mg/kg/day) or 75 ppm (˜10 mg/kg/day) chow dosing in SynCre⁺Pank1^{-/-}Pank2^{-/-} mice.

The human dosing requirement for target CoA elevation would need to take into account Compound (I) exposure via an oral dosing regimen and the sustained PD effect (CoA elevation) relative to the PK (blood Compound (I)). These considerations, alongside the trend toward some phenotypic rescue observed at 3 mpk, suggests that a lower dose could be effective, and that 75 ppm (10 mpk) is supratherapeutic.

### Example 3: Nonclinical Studies of Compound (I)

Pharmacokinetics of Compound (I) have been studied in mouse, rat, dog, and monkey. Following oral administration, Compound (I) was well absorbed with absolute oral bioavailability values for mouse, rat, dog, and monkey of 93.2%, 35.2%, 29.8%, and 17.6%, respectively. Following intravenous administration, systemic clearance of Compound (I) was relatively high (15.5, 32.6, 16.9, and 19.7 mL/min/kg for mouse, rat, dog, and monkey, respectively). Volume of distribution at steady state was low (0.614 to 1.02 L/kg), and t_{1/2} was relatively short (approximately 1 to 3 hours). Less than 2-fold accumulation of Compound (I) was observed after multiple doses in dogs and rats. Male rats had lower exposures than female rats at the same doses.

Both clinical formulations (Compound (I) compounded oral suspension and Compound (T) tablets) were evaluated in a crossover PK study in cynomolgus monkeys. Exposures obtained with the wet granulation tablet and the clinical suspension (10 and 200 mg/animal, respectively) of Compound (I) were quantitatively similar. Exposure of Compound (I), as defined by Cₘₐₓ, was close to dose proportional following oral gavage at 10 mg/animal and 200 mg/animal, either as a wet granulation tablet or as a clinical suspension. Exposure of Compound (I), as defined by AUC₀₋₂₄, appeared to be dose proportional following oral gavage at 10 mg/animal and 200 mg/animal either as a wet granulation tablet or clinical suspension.

### Example 4: A First-In-Human, Randomized, Placebo-Controlled, Single and Multiple Ascending Dose Study to Evaluate the Safety, Tolerability, Pharmacokinetics, and Pharmacodynamics of Compound (I) in Healthy Subjects and in Subjects with Propionic Acidemia or Methylmalonic Acidemia

### 4.1 Study Objectives

### Primary Objectives

The primary objectives for this study are as follows:
To evaluate the safety and tolerability of single and multiple doses of Compound (I) administered to healthy adult subjects and multiple doses of Compound (I) administered to patients with PA or MMA; and
To characterize the single- and multiple-dose PK of Compound (I) in healthy adult subjects.

### Secondary Objectives

The secondary objectives for this study are as follows:
To characterize the effect of food on the PK of a single dose of Compound (I) in healthy adult subjects;
To evaluate the effect of Compound (I) on ECG parameters including QTc, as measured at each PK time point, in healthy adult subjects;
To characterize the effects of Compound (I) on selected exploratory PD biomarkers of metabolism, including whole blood acetyl-CoA and plasma pantothenate concentrations, in healthy subjects and patients with PA or MMA;
To evaluate baseline and inter- and intrasubject variability of key PD biomarkers in healthy subjects and patients with PA or MMA; and
To characterize the multiple dose PK of Compound (I) in patients with PA or MMA.

### 4.2 Study Design

This is a Phase 1 first-in-human study to evaluate the safety, tolerability, PK, and PD of Compound (I) when administered as single and multiple oral doses to healthy adult subjects.

This study will also evaluate multiple doses of Compound (I) in patients with PA or MMA.

### Single Ascending Dose (SAD) Cohorts

The SAD portion of the study will have up to 8 cohorts and up to a total of 64 healthy subjects (8 subjects per cohort).

This portion of the study will consist of a screening period, Check-in, a treatment period, and an EOS visit. Subjects will be randomly assigned to receive Compound (I) or placebo in a 6:2 ratio. Sentinel dosing will be employed in all SAD cohorts (except for Treatment Period 2 of the FE cohort), whereby the first 2 subjects (1 active and 1 placebo) will be dosed at least 48 hours before the remaining 6 subjects at that level are dosed. Initiation of dosing of the remaining 6 subjects will depend on an initial safety review (e.g., clinical laboratory results, vital signs, ECGs, and AEs from the preceding 48 hours) by the investigator indicating that administration of the study treatment was safe and well tolerated in the sentinel subjects.

Continuous cardiac telemetry and Holter monitoring will be obtained as indicated in the SOE (Table 6).

Subjects will receive a single dose of Compound (I) on Day 1. The starting dose of Compound (I) compounded oral suspension will be 3 mg and the planned doses for subsequent cohorts will be 10, 30, 60, 120, 200, and 300 mg. The dose levels may be adjusted by the SRC. The SRC will review at least 24 hours of plasma PK data and at least 48 hours of safety data (including clinical laboratory results, vital signs, ECGs, and AEs) after study drug dosing from a minimum of 6 subjects (which may include sentinel subjects) in the preceding dose level before a decision is made to increase to the next dose level. The actual doses given during the SAD portion of the study were 3, 10, 30, 60, 100, and 120 mg QD.

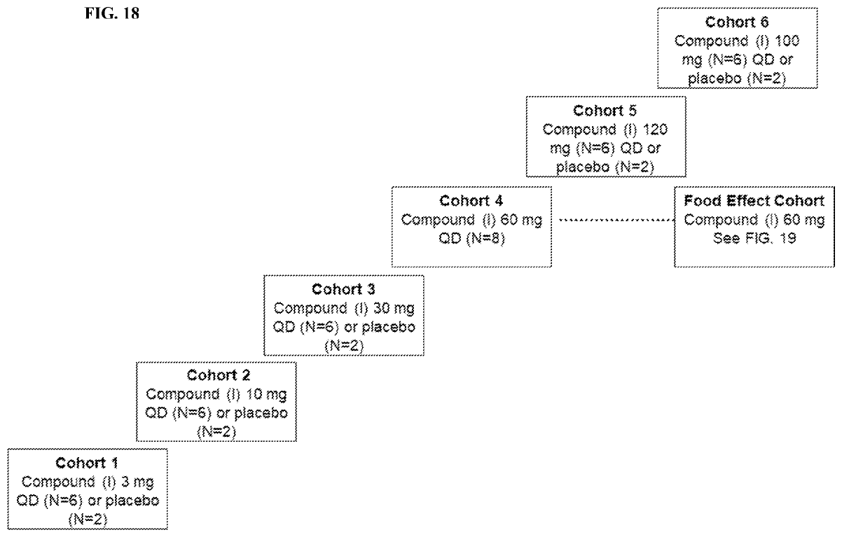

The study design for the SAD portion of the study is provided in FIG. 18.

Subjects will fast overnight (nothing to eat or drink except water) for at least 10 hours before study drug administration. Subjects will remain fasted for 4 hours after dosing with study drug. Water is permitted ad libitum except for 1 hour before and after dosing.

Subjects will be confined to the clinical unit from Day −1 until discharge on Day 3. From Cohort 2 onwards, the confinement duration may be updated to at least 3 half-lives up to a maximum of Day 6 based on Cohort 1 data. An EOS visit will occur on Day 10 or approximately 5 half-lives up to Day 30 (whichever is longer).

Blood and urine for PK and PD will be obtained and safety assessments will be performed. Table 6 summarizes study activities and assessments for the SAD portion of the study.

The length of participation for subjects in the SAD cohort is approximately 61 days; up to 30 days for the screening period, up to 7 days in house and up to 24 days until the EOS visit.

TABLE 6
Schedule of Events - Single Ascending Dose Cohorts

| | Phase | | | | | |
|---|---|---|---|---|---|---|
| | Screening | Check-in | Treatment Period | | | EOS/ED ^{(b)} |
| | Day | | | | | |
| Procedure^{(a)} | −30 to −2 | −1 | 1 | 2 | 3 | 10 (±2) |
| Admission to clinic | | X | | | | |
| Discharge from clinic^{(c)} | | | | | X | |
| Clinic visit | X | | | | | X |
| Informed consent | X | | | | | |
| Demographics | X | | | | | |
| Serology^{(d)} | X | | | | | |
| Serum FSH^{(e)} | X | | | | | |
| Inclusion/exclusion criteria | X | X | | | | |
| Medical history | X | X | | | | |
| Height, weight, and BMI^{(f)} | X | X | | | | X |
| Physical examination^{(g)} | X | X | | | X | X |
| Vital sign measurements^{(h)} | X | X | X | X | X | X |
| 12-Lead ECG⁽ⁱ⁾ | X | X | X | | X | X |
| Continuous cardiac telemetry^{(j)} | | X | X | X | | |
| 12-Lead Holter recording^{(k)} | | | X | X | | |
| Serum chemistry and urinalysis^{(l)} | X | X | | X | | X |
| Hematology^{(l)} | X | X | | X | X | X |
| Drug, alcohol, and cotinine screen^{(m)} | X | X | | | | |
| Genotyping⁽ⁿ⁾ | | | X | | | |
| Pregnancy test^{(o)} | X | X | | | | |
| Randomization^{(p)} | | | X | | | |
| Study drug administration^{(q)} | | | X | | | |
| PK blood sample collection^{(r)} | | | X | X | X | |
| PK urine sample collection^{(s)} | | | X | X | X | |
| PD biomarker sample collection^{(t)} | | | X | X | X | |
| Fasting period^{(u)} | | X | X | | | |
| Nonfasting period^{(v)} | | | | X | X | X |

| | |
|---|---|
| AEs^{(w)} | [symbol] X [symbol] |

| | |
|---|---|
| Prior/concomitant medications | [symbol] X [symbol] |

^{(a)}When procedures overlap or occur at the same time point, all blood draws will follow vital signs or ECGs, and PK sampling will be timed to occur last and as close to the scheduled time window as possible.
^{(b)} The EOS visit will occur on Day 10 or approximately 5 half-lives up to Day 30 (whichever is longer).
^{(c)}Discharge will occur following the 48-hour PK sample collection and completion of safety assessments.
^{(d)}Serology testing will include hepatitis B surface antigen, hepatitis C virus antibody, and HIV types 1 and 2 antibodies.
^{(e)}A serum FSH test may be performed for females at Screening to confirm postmenopausal status.
^{(f)}Height and weight will be measured and BMI calculated at Screening only. Only weight will be measured at Check-in and EOS.
^{(g)}A full physical examination will be performed at Screening (at minimum, assessment of skin, head, ears, eyes, nose, throat, neck, thyroid, lungs, heart, cardiovascular, abdomen, lymph nodes, and musculoskeletal system/extremities). A brief physical examination will be performed at Check-in, discharge from clinic, and EOS (at minimum, assessment of skin, lungs, cardiovascular system, and abdomen [liver and spleen]). Interim physical examinations may be performed at the discretion of the investigator, if necessary, to evaluate AEs or clinical laboratory abnormalities.
^{(h)}Vital signs will include systolic and diastolic blood pressure, pulse rate, respiratory rate, and body temperature (temperature once daily with first set of vital signs for the day). Vital signs will be measured after the subject has been resting quietly in the seated position for at least 5 minutes and will be measured at predose (within 90 minutes) and at 4 hours postdose (±30 minutes) on Day 1, and in the morning upon awakening on other days. A single repeat measurement is permitted at both Screening and Check-in for eligibility determination.
⁽ⁱ⁾Single 12-lead ECG recordings will be made at stipulated time points after the subject has been in the supine position for at least 5 minutes. A single repeat measurement is permitted at both Screening and Check-in for eligibility determination. Measurements of the following intervals will be reported: RR interval, PR interval, QRS width, QT interval, and QTcF. Assessments will include comments on whether the tracings are normal or abnormal; rhythm; presence of arrhythmia or conduction defects; morphology; any evidence of myocardial infarction; or ST-segment, T-wave, and U-wave abnormalities. On Day 1, ECG readings taken at predose (within 90 minutes) and at 4 and 8 hours postdose.
^{(j)}Continuous cardiac telemetry will be obtained starting on Day −1 and continue until at least 24 hours after dosing.
^{(k)}The Holter monitor will be started at least 2 hours before dosing and will continue until at least 24 hours after dosing. Details regarding Holter monitoring are provided in Section 6.3.1 of the clinical protocol.
^{(l)}A complete list of assessments is provided in Section 6.4.2 of the clinical protocol. Blood and urine samples will be collected under fasted conditions and prepared per the clinic's standard procedures. A single repeat measurement is permitted at both Screening and Check-in for eligibility determination.
^{(m)}Urine or blood drug/alcohol/cotinine screen will occur per the clinic's standard procedures.
⁽ⁿ⁾Genotyping for CYP2C19 and CYP2D6. Test may be done at any time during the study.
^{(o)}All women. Urine or serum, per site policy.
^{(p)}Subjects will be randomly assigned to receive Compound (I) or placebo.
^{(q)}The time of study drug dosing will be called “0” hour. The study drug will be administered with 240 mL of room temperature water. Up to an additional 240 mL of water will be allowed, if necessary, to aid in swallowing of the study drugs. Subjects will maintain an upright (i.e., seated or standing) position for at least 4 hours after dosing.
^{(r)}Blood samples for PK analysis will be collected at predose (within 120 minutes prior to study drug dosing) and at 0.5, 1, 1.5, 2, 3, 4, 6, 8, 12, 24 (Day 2), 36 (Day 2), and 48 (Day 3) hours postdose. The PK sampling times and duration of sampling for Cohorts 2 and beyond may be adjusted up to a maximum of 8 additional blood draws depending on the half-life of Compound (I).
^{(s)}Urine samples for PK analysis will be collected at predose (within 2 hours prior to study drug dosing) and at the following intervals postdose: 0 to 6, 6 to 12, 12 to 24, 24 to 36, and 36 to 48 hours.
^{(t)}Blood for PD biomarker sampling will be collected at predose (within 120 minutes prior to study drug dosing) and at 1, 2, 4, 8, 12, 24 (Day 2) and 48 (Day 3) hours postdose. The PD biomarker sampling times and duration of sampling may be adjusted depending on the half-life, up to a maximum of 8 additional blood draws. Pharmacodynamic biomarkers will include whole blood acetyl-CoA, plasma pantothenate, and other exploratory biomarkers.
^{(u)}During fasting periods, subjects must have nothing to eat or drink except water from 10 hours prior to study drug dosing until 4 hours after dosing. Water is permitted as desired except for the period 1 hour before and 1 hour after administration of study drug (other than as permitted for study drug dosing).
^{(v)}During nonfasting periods, subjects will receive standardized meals per the clinic's standard procedures.
^{(w)}Adverse events will be assessed from the time of study drug dosing until EOS and must be followed until they are resolved, stable, or judged by the investigator to be not clinically significant.

### Food Effect Cohort

The SAD portion of the study will include an active treatment FE cohort of 8 healthy subjects. The 60 mg cohort (SAD Cohort 4) will participate in the FE portion.

This portion of the study will consist of a screening period, check-in days, 2 treatment periods, a washout period, and an EOS visit. Sentinel dosing will be employed in Treatment Period 1. No sentinel dosing will be employed in Treatment Period 2. All subjects will receive 2 doses of study drug in a crossover manner, separated by a washout period of 7 days or 5 half-lives up to 21 days (whichever is longer).

Subjects will be randomly assigned to receive Compound (I) under fasted conditions or following a high-calorie breakfast in 1 of 2 treatment sequences as follows:

| Sequence | Period 1 | Period 2 |
|---|---|---|
| 1 | Fed | Fasted |
| 2 | Fasted | Fed |

Continuous cardiac telemetry, Holter recording, and fasting conditions are indicated in the SOE (Table 7).

Drug administration under fasted conditions is described herein. When study drug is given under fed conditions, subjects will fast overnight (nothing to eat or drink except water) for at least 10 hours before study drug administration. Study drug will then be administered along with a standardized high-calorie (approximately 800 to 1000 calories) breakfast. The meal will be ingested entirely within 30 minutes, and study drug will be administered 30 minutes after the start of the meal.

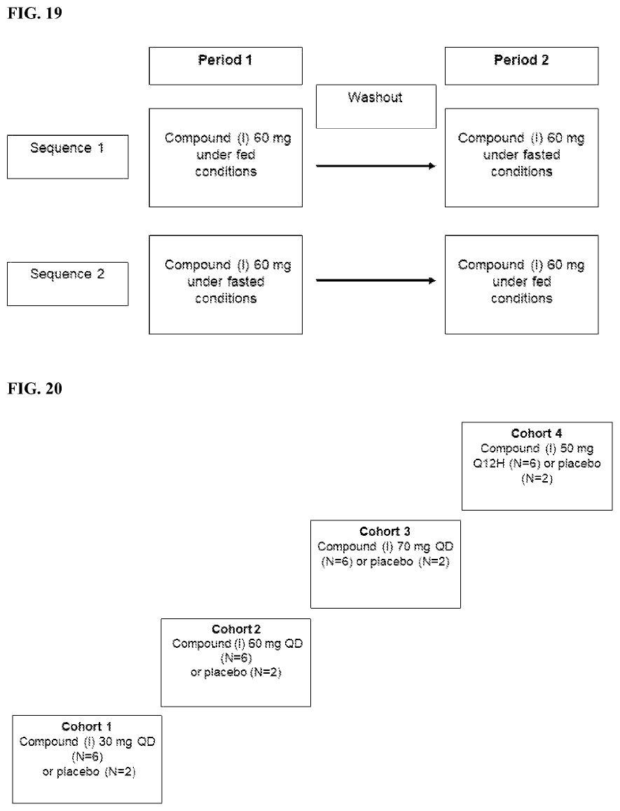

The study design for the FE portion of the study is provided in FIG. 19.

In Treatment Period 1, subjects will be confined to the clinical unit from Day −1 until discharge on Day 3. Following the washout period after Treatment Period 1, subjects will return to the clinical unit on Treatment Period 2 Day −1 and will be discharged on Day 3. An EOS visit will occur on Day 10 or approximately 5 half-lives up to Day 30 (whichever is longer) after dosing in Treatment Period 2.

Blood and urine for P and PD will be obtained, and safety assessments will be performed. Table 7 summarizes study activities and assessments for the FE portion of the study.

The length of participation for subjects in the FE cohort is approximately 88 days; up to 30 days for the screening period, up to 7 days in house in each period; up to 21 days of washout between periods, and up to 30 days until the EQS visit.

TABLE 7
Schedule of Events - Food Effect Cohort

| | Phase | | | | | |
|---|---|---|---|---|---|---|
| | Screening | Check-in | Treatment Periods 1 and 2 | | | EOS/ED^{(b)} |
| | Day | | | | | |
| Procedure^{(a)} | −30 to −2 | −1 | 1 | 2 | 3 | 10 (±2) |
| Admission to clinic | | X | | | | |
| Discharge from clinic^{(c)} | | | | | X | |
| Clinic visit | X | | | | | X |
| Informed consent | X | | | | | |
| Demographics | X | | | | | |
| Serology^{(d)} | X | | | | | |
| Serum FSH^{(c)} | X | | | | | |
| Inclusion/exclusion criteria | X | X | | | | |
| Medical history | X | X | | | | |
| Height, weight, and BMI^{(f)} | X | X | | | | X |
| Physical examination^{(g)} | X | X | | | X | X |
| Vital sign measurements^{(h)} | X | X | X | X | X | X |
| 12-Lead ECG⁽ⁱ⁾ | X | X | X | | X | X |
| Continuous cardiac telemetry^{(j)} | | X | X | X | | |
| 12-Lead Holter recording^{(k)} | | | X | X | | |
| Clinical laboratory testing^{(l)} | X | X | | | X | X |
| Drug/alcohol/cotinine screen^{(m)} | X | X | | | | |
| Pregnancy test⁽ⁿ⁾ | X | X | | | | |
| Randomization^{(o)} | | | X | | | |
| Study drug administration^{(p)} | | | X | | | |
| High-calorie breakfast^{(q)} | | | X | | | |
| PK blood sample collection^{(r)} | | | X | X | X | |
| PK urine sample collection^{(s)} | | | X | X | X | |
| PD biomarker sample collection^{(t)} | | | X | X | X | |
| Fasting period^{(u)} | | X | X | | | |
| Nonfasting period^{(v)} | | | | X | X | X |

| | |
|---|---|
| AEs^{(v)} | [symbol] X [symbol] |

| | |
|---|---|
| Prior/concomitant medications | [symbol] X [symbol] |

^{(a)}When procedures overlap or occur at the same time point, all blood draws will follow vital signs or ECGs, and PK sampling will be timed to occur last and as close to the scheduled time window as possible.
^{(b)}An EOS visit will occur after completion of Treatment Period 2 only. The EOS visit will occur on Day 10 or approximately 5 half-lives up to Day 30 (whichever is longer).
^{(c)}Discharge will occur following the 48-hour PK sample collection and completion of safety assessments. There will be a washout period of 7 days or 5 half-lives up to 21 days (whichever is longer) between dosing in Treatment Period 1 and dosing in Treatment Period 2.
^{(d)}Serology testing will include hepatitis B surface antigen, hepatitis C virus antibody, and HIV virus types 1 and 2 antibodies.
^{(e)}A serum FSH test may be performed for females at Screening to confirm postmenopausal status.
^{(f)}Height and weight will be measured and BMI calculated at Screening only. Only weight will be measured at Check-in and EOS.
^{(g)}A full physical examination will be performed at Screening (at minimum, assessment of skin, head, ears, eyes, nose, throat, neck, thyroid, lungs, heart, cardiovascular, abdomen, lymph nodes, and musculoskeletal system/extremities). A brief physical examination will be performed at Check-in, discharge from clinic, and EOS (at minimum, assessment of skin, lungs, cardiovascular system, and abdomen [liver and spleen]). Interim physical examinations may be performed at the discretion of the investigator, if necessary, to evaluate AEs or clinical laboratory abnormalities.
^{(h)}Vital signs will include systolic and diastolic blood pressure, pulse rate, respiratory rate, and body temperature (temperature once daily with first set of vital signs for the day). Vital signs will be measured after the subject has been resting quietly in the seated position for at least 5 minutes and will be measured at predose (within 90 minutes) and 4 hours postdose (±30 minutes) on Day 1 and in the morning upon awakening on other days. A single repeat measurement is permitted at both Screening and Check-in for eligibility determination.
⁽ⁱ⁾Single 12-lead ECG recordings will be made at stipulated time points after the subject has been in the supine position for at least 5 minutes. A single repeat measurement is permitted at both Screening and Check-in for eligibility determination. Measurements of the following intervals will be reported: RR interval, PR interval, QRS width, QT interval, and QTcF. Assessments will include comments on whether the tracings are normal or abnormal; rhythm; presence of arrhythmia or conduction defects; morphology; any evidence of myocardial infarction; or ST-segment, T-wave, and U-wave abnormalities. On Day 1, ECG readings will be taken at predose (within 90 minutes) and at 4 and 8 hours postdose.
^{(j)}Continuous cardiac telemetry will be obtained in Treatment Period 1 only and will start on Day −1 and continue until at least 24 hours after dosing.
^{(k)}The Holter monitor will be started at least 2 hours before dosing and will continue until at least 24 hours after dosing when study drug is given in the fasted state only. Details regarding Holter monitoring are provided in Section 6.3.1 of the clinical protocol.
^{(l)}A complete list of assessments is provided in Section 6.4.2 of the clinical protocol. Blood and urine samples will be collected under fasted conditions and prepared per the clinic's standard procedures. A single repeat measurement is permitted at both Screening and Check-in for eligibility determination.
^{(m)}Urine or blood drug/alcohol/cotinine screen will occur per the clinic's standard procedures.
⁽ⁿ⁾All women. Urine or serum, per site policy.
^{(o)}Subjects will be randomly assigned to a treatment sequence (fed/fasted or fasted/fed).
^{(p)}The time of study drug dosing will be called “0” hour. The study drug will be administered with 240 mL of room temperature water. Up to an additional 240 mL of water will be allowed, if necessary, to aid in swallowing of the study drug. Subjects will maintain an upright (i.e., seated or standing) position for at least 4 hours after dosing.
^{(q)}When receiving study drug in the fed condition, subjects will receive a high-calorie breakfast approximately 30 minutes prior to study drug administration. The meal will be ingested entirely within 30 minutes and study drug will be administered 30 minutes after the start of the meal.
^{(r)}Blood samples for PK analysis will be collected at predose (within 120 minutes prior to study drug dosing) and at 0.5, 1, 1.5, 2, 3, 4, 6, 8, 12, 24 (Day 2), 36 (Day 2), and 48 (Day 3) hours postdose. The PK sampling times and duration of sampling may be adjusted up to a maximum of 8 additional blood draws depending on the half-life of Compound (I).
^{(s)}Urine samples for PK analysis will be collected at predose (within 2 hours prior to study drug dosing) and at the following intervals after dosing: 0 to 6, 6 to 12, 12 to 24, 24 to 36, and 36 to 48 hours.
^{(t)}Blood for PD biomarker sampling will be collected at predose (within 120 minutes prior to study drug dosing) and at 1, 2, 4, 8, 12, 24 (Day 2), and 48 (Day 3) hours postdose. The PD biomarker sampling times and duration of sampling may be adjusted depending on the half-life, up to a maximum of 8 additional blood draws. Biomarkers will include whole blood acetyl-CoA, plasma pantothenate, and other exploratory biomarkers.
^{(u)}During fasting periods (including when study drug is administered in the fasted condition), subjects must have nothing to eat or drink except water from 10 hours prior to study drug dosing until 4 hours after dosing. Water is permitted as desired except for the period 1 hour before and 1 hour after administration of study drug (other than as permitted for study drug dosing).
^{(v)}During nonfasting periods, subjects will receive standardized meals per the clinic's standard procedures which will be scheduled at the same time in each period of the study.
^{(w)}Adverse events will be assessed from the time of study drug dosing until EOS and must be followed until they are resolved, stable, or judged by the investigator to be not clinically significant.

### Multiple Ascending Dose (MAD) Cohorts

The MAD portion of the study will have up to 6 cohorts and up to a total of 48 healthy subjects (8 subjects per cohort).

The first MAD cohort may be enrolled after the SRC has reviewed a minimum of 48 hours of safety data and 24 hours of PK data from at least 2 SAD cohorts. The starting dose and dosing frequency for the initial MAD cohort (e.g., once daily, twice daily) will be determined by the SRC before initiation of the MAD enrollment. The starting dose will be the one with a predicted exposure at steady state that has previously been well tolerated after a single dose, as determined by the SRC. Any dose used in the MAD portion of the study will not be greater than the highest tested tolerable dose from SAD portion of the study.

This portion of the study will consist of a screening period, Check-in, a treatment period, and EOS visit. Subjects will be randomly assigned to receive Compound (I) or placebo in a 6:2 ratio.

Following the screening period, eligible subjects will be admitted to the clinical unit on Day 1. All subjects will receive the study drug for 7 days starting on Day 1. If the dosing frequency is greater than once daily, then the last dose of the study drug will be the morning dose on Day 7. Dosing frequency and any instructions regarding meal intake will depend on data from the SAD cohorts and will be provided in a separate document. The dosing duration may be increased to up to 14 days to allow for achievement of 3 half-lives. The maximum stay in the clinical unit will not exceed 19 days (including 14 days of dosing and 96-hours postdose for PK determinations). Subjects will be discharged from the clinical unit at least 48 hours after the last dose of study drug (Day 9). Subjects will return for an EOS visit on Day 15 or up to approximately 5 half-lives up to a maximum of Day 42 (whichever is longer).

The tentative starting dose for this portion of the study will be 30 mg daily. Dose escalation for Cohort 2 and beyond will be based on the safety, tolerability, and PK of the previous cohort. The SRC will review at least 24 hours of plasma PK data and at least 48 hours of safety data (including clinical laboratory results, vital signs, ECGs, and AEs) after the final study drug dose from a minimum of 6 subjects in the preceding dose level before a decision is made to increase to the next dose level. Planned doses for subsequent cohorts are 60, 120, 200, and 300 mg daily; the maximum dose increment between cohorts will be no more than 2 times the previous dose. The actual doses given during the MAD portion of the study were 30, 60, and 70 mg daily; and 50 mg BID (every 12 hours).

The study design for the MAD portion of the study is presented in FIG. 20.

The dosing frequency for the MAD cohorts (e.g., once daily, every 12 hours) will be determined by the SRC. If dosing frequency is greater than once daily, the total tentative daily doses shown above will be divided (e.g., divided by 2 for every 12-hour dosing). If dosing frequency is greater than once daily, dosing of MAD cohorts will begin once the FE data are available; any deviation regarding the fasting requirements in this case will be provided in a separate document.

Holter monitoring will be obtained as indicated in the SOE. Blood and urine for PK and PD will be obtained and safety assessments will be performed. In addition, Cohort 3 (70 mg cohort) will have CSF obtained via lumbar puncture for PK assessment. Table 8 summarizes study activities and assessments for the MAD portion of the study.

The length of participation for subjects in the MAD portion of the study is approximately 84 days; up to 30 days for the screening period, up to 19 days in house, and up to 35 days after the last dose of study drug until the EOS visit.

TABLE 8
Schedule of Events - Multiple Ascending Dose Cohorts

| | Phase | | | | | | | | | | | |
|---|---|---|---|---|---|---|---|---|---|---|---|---|
| | Screening | Check-in | Treatment Period | | | | | | | | | EOS/ED |
| | Day | | | | | | | | | | | |
| Procedure^{(a)} | −30 to −2 | −1 | 1 | 2 | 3 | 4 | 5 | 6 | 7 | 8 | 9 | 15 (±2) |
| Admission to clinic | | X | | | | | | | | | | |
| Discharge from clinic^{(b)} | | | | | | | | | | | X | |
| Clinic visit | X | | | | | | | | | | | X |
| Informed consent | X | | | | | | | | | | | |
| Demographics | X | | | | | | | | | | | |
| Serology^{(c)} | X | | | | | | | | | | | |
| Serum FSH^{(d)} | X | | | | | | | | | | | |
| Inclusion/exclusion criteria | X | X | | | | | | | | | | |
| Medical history | X | X | | | | | | | | | | |
| Height, weight, and BMI^{(e)} | X | X | | | | | | | | | | X |
| Physical examination^{(f)} | X | X | | | | | | | | | X | X |
| Vital sign measurements^{(g)} | X | X | X | X | X | X | X | X | X | X | X | X |
| 12-Lead ECG^{(h)} | X | X | X | | X | | X | | X | | X | X |
| 12-Lead Holter recording⁽ⁱ⁾ | | | X | X | | | | | X | X | | |
| Serum chemistry and | X | X | | | | X | | | | | X | X |
| urinalysis^{(j)} | | | | | | | | | | | | |
| Hematology^{(j)} | X | X | | | | X | X | X | X | | X | X |
| Drug/alcohol/cotinine | X | X | | | | | | | | | | |
| screen^{(k)} | | | | | | | | | | | | |
| Pregnancy test^{(l)} | X | X | | | | | | | | | | |
| Genotyping^{(m)} | | | X | | | | | | | | | |
| Ocular examination⁽ⁿ⁾ | | X | | | | | | | | | | X |
| Randomization^{(o)} | | | X | | | | | | | | | |
| Study drug administration^{(p)} | | | X | X | X | X | X | X | X | | | |
| PK blood sample | | | X | X | X | X | X | X | X | X | X | |
| collection^{(q)} | | | | | | | | | | | | |
| PK urine sample | | | X | | | | | | X | X | | |
| collection^{(r)} | | | | | | | | | | | | |
| PD biomarker sample | | | X | X | X | X | X | X | X | X | X | |
| collection^{(s)} | | | | | | | | | | | | |
| Lumbar puncture^{(t)} | | | | | | | | X | | | | |
| AEs^{(u)} | | | [symbol] X [symbol] | | | | | | | | | |
| Prior/concomitant | [symbol] X [symbol] | | | | | | | | | | | |
| medications | | | | | | | | | | | | |

^{(a)}When procedures overlap or occur at the same time point, all blood draws will follow vital signs or ECGs, and PK sampling will be timed to occur last and as close to the scheduled time window as possible.
^{(b)}Discharge will occur following the 48-hour PK sample collection after the last dose of the study drug and completion of safety assessments. In case the dosing duration is increased to 14 days^{(p)} the maximum stay in the clinic will not exceed 19 days (including 14 days of dosing and 96 hours postdose PK sample collection). Similarly, the EOS visit may be adjusted to at least 5 half-lives, up to a maximum of Day 42.
^{(c)}Serology testing will include hepatitis B surface antigen, hepatitis C virus antibody, and HIV 1 and 2 antibodies.
^{(d)}A serum FSH test may be performed for females at Screening to confirm postmenopausal status.
^{(e)}Height and weight will be measured and BMI calculated at Screening only. Only weight will be measured at Check-in and EOS.
^{(f)}A full physical examination will be performed at Screening (at minimum, assessment of skin, head, ears, eyes, nose, throat, neck, thyroid, lungs, heart, cardiovascular, abdomen, lymph nodes, and musculoskeletal system/extremities). A brief physical examination will be performed at Check-in, discharge from clinic, and EOS (at minimum, assessment of skin, lungs, cardiovascular system, and abdomen [liver and spleen]). Interim physical examinations may be performed at the discretion of the investigator, if necessary, to evaluate AEs or clinical laboratory abnormalities.
^{(g)}Vital signs will include systolic and diastolic blood pressure, pulse rate, respiratory rate, and body temperature (temperature once daily with first set of vital signs for the day). Vital signs will be measured after the subject has been resting quietly in the seated position for at least 5 minutes and will be measured at predose (within 90 minutes) and 4 hours postdose (±30 minutes) relative to the morning dose on Day 1 and in the morning upon awakening on other days. A single repeat measurement is permitted at both Screening and Check-in for eligibility determination.
^{(h)}Single 12-lead ECG recordings will be made at stipulated time points after the subject has been in the supine position for at least 5 minutes. A single repeat measurement is permitted at both Screening and Check-in for eligibility determination. Measurements of the following intervals will be reported: RR interval, PR interval, QRS width, QT interval, and QTcF. Assessments will include comments on whether the tracings are normal or abnormal; rhythm; presence of arrhythmia or conduction defects; morphology; any evidence of myocardial infarction; or ST-segment, T-wave, and U-wave abnormalities. Electrocardiogram time points: predose (within 90 minutes) and at 4 and 8 hours after the morning dose on Day 1; at 2 hours after the morning dose on other days; and in the morning if not a dosing day.
⁽ⁱ⁾Continuous 12-lead Holter monitor will be performed starting at least 2 hours before dosing on Day 1 until at least 24 hours after the first dose. The 12-lead Holter recording will be repeated starting within 1 hour prior to the last dose until at least 24 hours after the last dose. Details regarding Holter monitoring are provided in Section 6.3.1 of the clinical protocol.
^{(j)}A complete list of assessments is provided in Section 6.4.2 of the clinical protocol. Blood and urine samples will be collected under fasted conditions and prepared per the clinics standard procedures. A single repeat measurement is permitted at both Screening and Check-in for eligibility determination.
^{(k)}Urine or blood drug/alcohol/cotinine screen will occur per the clinic's standard procedures.
^{(l)}All women. Urine or serum, per site policy.
^{(m)}Genotyping for CYP2C19 and CYP2D6. Test may be done at any time during the study.
⁽ⁿ⁾Ocular examinations to include best corrected visual acuity, slit lamp testing, and corneal fluorescein staining. Corneal topography may be performed if clinically indicated per assessment of the ophthalmologist.
^{(o)}Subjects will be randomly assigned to receive Compound (I) or placebo.
^{(p)}The study drug will be administered with 240 mL of room temperature water. Up to an additional 240 mL of water will be allowed, if necessary, to aid in swallowing of the study drugs. Subjects will maintain an upright (i.e., seated or standing) position for at least 4 hours after dosing. Subjects are encouraged to remain upright (i.e., sitting or standing) after the second dose when BID dosing occurs. The dosing duration may be increased to up to 14 days to allow for achievement of 3 half-lives. When BID dosing occurs, the final dose will be administered in the morning of Day 7.
^{(q)}Timing of samples is relative to the morning dose when BID dosing occurs. Blood samples for PK analysis will be collected at the following time points - Day 1: predose (within 120 minutes prior to study drug dosing) and at 0.5, 1, 1.5, 2, 3, 4, 6, 8, and 12 hours postdose; Days 2 through 6: predose (within 30 minutes prior to study drug dosing); Day 7: predose (within 30 minutes prior to study drug dosing) and at 0.5, 1, 1.5. 2, 3, 4, 6, 8, and 12 hours after the last dose; at 24 and 36 hours (Day 8) and 48 hours (Day 9) postdose. The PK sampling times and duration of sampling may be adjusted depending on the half-life, up to a maximum of 8 additional blood draws.
^{(r)}Timing of samples is relative to the first dose when BID dosing occurs. Urine samples for PK analysis will be collected at predose (within 2 hours prior to study drug dosing) on Day 1 and at the following intervals after dosing on Day 7: 0 to 6, 6 to 12, and 12 to 24 hours.
^{(s)}Timing of samples is relative to the first dose when BID dosing occurs. Blood samples for PD biomarker analysis will be collected at following time points - Day 1: predose (within 120 minutes prior to study drug dosing) and at 4, 8, and 12 hours after dosing; Days 2 through 6: predose (within 30 minutes prior to study drug dosing); Day 7: predose (within 30 minutes prior to study drug dosing) and at 4, 8, 12, 24 (Day 8), and 48 (Day 9) hours postdose. The PD biomarker sampling times and duration of sampling may be adjusted depending on the half-life, up to a maximum of 8 additional blood draws. Biomarkers will include whole blood acetyl-CoA, plasma pantothenate, and other exploratory biomarkers.
^{(t)}A CSF sample will be collected in in Cohort 3 (70 mg) participants around Tₘₐₓ (to be determined based on prior cohorts); a PK sample must also be obtained immediately after the lumbar puncture unless the lumbar puncture coincides with a scheduled PK sample collection.
^{(u)}Adverse events will be assessed from the time of study drug dosing until EOS and must be followed until they are resolved, stable, or judged by the investigator to be not clinically significant.

### Propionic Acidemia (PA) and Methylmalonic Acidemia (MMA) Cohort

Compound (I) will be assessed in an open-label cohort of up to 16 patients (up to 8 patients each with PA or MMA).

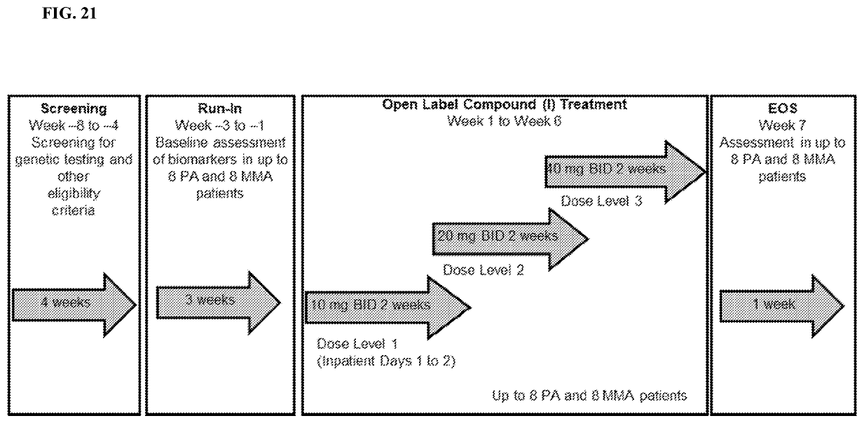

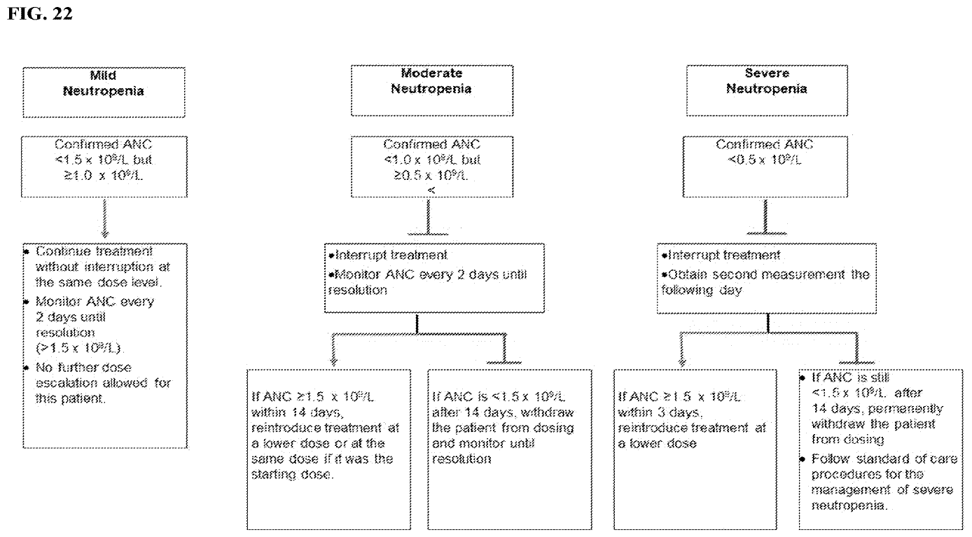

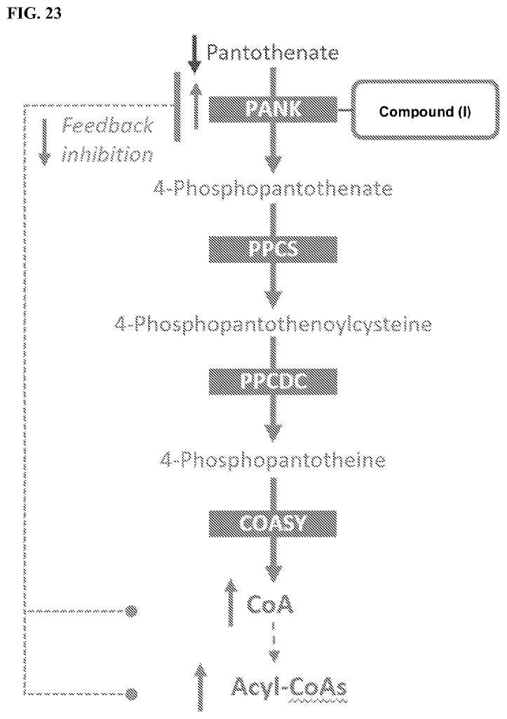

Three dose levels are planned for each patient in the PA/MMA cohort. The 3 dose levels are 10 mg BID (Dose Level 1), 20 mg BID (Dose Level 2), and 40 mg BID (Dose Level 3). Intrapatient dose escalation will be reviewed on a patient-by-patient basis with the Investigator and Sponsor based on safety data and may vary from the planned doses (FIG. 21), but will not exceed 2,370 ng/mL for Cₘₐₓ or 10,700 h ng/mL for AUC₀₋₂₄, the steady state exposures observed at the 8 mg/kg/day NOAEL in the 13-week GLP toxicology study in female rats, which represent the most sensitive sex and species.

This portion of the study will consist of a screening period, an outpatient run-in period, an inpatient treatment period, outpatient dosing days, and an EOS visit.

Following the screening period, eligible patients will participate in a 3-week Run-In Period for baseline assessment of PD biomarkers. Patients will visit the clinical unit on a weekly basis from run-in Week −3 to run-in Week −1. Patients will be admitted to the clinical unit on Day −1 and BID dosing will start on Day 1. The first 3 doses of study drug will be administered in the clinical unit. Patients will be discharged from the clinical unit on Day 2 at least 24 hours after the first dose of study drug (patients will take the evening Day 2 dose at home). Twice daily dosing (at least 10 hours apart) with study drug will continue for up to 42 days.

A safety laboratory visit to collect a blood sample for hematology will occur on Day 4. After the clinic visit on Day 7, patients will have safety laboratory visits every 2 to 3 days (i.e., 2 times per week) between scheduled clinic visits. Hematology tests will be performed at these visits. The frequency of this testing may be increased as per investigator discretion. Home visits may be used for these assessments.

Patients will return for outpatient visits as indicated in the SOE (Table 9). The last dose of the study drug is planned to be the evening dose on Day 42.

For the PA/MMA cohort, a diet diary will be used to maintain a daily diet record for the 3 days prior to the visits indicated in Table 9. The diet diary will be provided by the clinical site with instructions on how the form should be filled out, and will be reviewed with the patient and/or caregiver by clinical site staff at each indicated visit.

The study design for the PA and MMA cohort is provided in FIG. 21.

Blood for PK and PD will be obtained and safety assessments will be performed. Table 9 summarizes study activities and assessments for the PA/MMA cohort.

The length of participation for patients in the PA/MMA cohort is approximately 14 weeks. The screening period is approximately 4 weeks, the run-in period is 3 weeks, the dosing period is up to 6 weeks (including up to 2 days in house for initial dosing), and the EOS visit is 1 week after the last dose of Compound (I).

TABLE 9
Schedule of Events - Propionic Acidemia and Methylmalonic Acidemia Cohort

| | Phase | | | | | | | | | | | | | |
|---|---|---|---|---|---|---|---|---|---|---|---|---|---|---|
| | Screening | Run-in | | | Check-in | Treatment Period | | | | | | | | EOS/ED |
| | Week | | | | | | | | | | | | | |
| | −8 to −4 | −3 | −2 | −1 | 0 | 1 | | | 2 | 3 | 4 | 5 | 6 | 7 |
| | Day | | | | | | | | | | | | | |
| | −56 to −28 | −21 | −14 | −7 | −1 | 1 | 2 ^{(a)} | 7 ^{(a)} | 14 ^{(a)} | 21 ^{(a)} | 28 ^{(a)} | 35 ^{(a)} | 42 ^{(a)} | 49 |
| | Window period | | | | | | | | | | | | | |
| Procedure^{(b)} | — | ±3 | ±3 | ±3 | — | — | — | — | ±3 | ±3 | ±3 | ±3 | ±3 | +7 |
| Inpatient admission | | | | | X | X | | | | | | | | |
| Discharge after inpatient | | | | | | | X | | | | | | | |
| stay^{(c)} | | | | | | | | | | | | | | |
| Outpatient clinic visit | X | X | X | X | | | | X | X | X | X | X | X | X |
| Informed consent | X | | | | | | | | | | | | | |
| Genetic testing to confirm | X | | | | | | | | | | | | | |
| PA/MMA^{(d)} | | | | | | | | | | | | | | |
| Laboratory samples to | X | | | | | | | | | | | | | |
| verify eligibility^{(e)} | | | | | | | | | | | | | | |
| Demographics | | | | | | | | | | | | | | |
| Serology^{(f)} | X | | | | | | | | | | | | | |
| Serum FSH^{(g)} | X | | | | | | | | | | | | | |
| Inclusion/exclusion criteria | X | | | | X | | | | | | | | | |
| Medical history | X | X | | | | | | | | | | | | |
| Height, weight, and BMI^{(h)} | X | | | | X | | | | | | | | | X |
| Physical examination⁽ⁱ⁾ | X | | | | X | | | | | | | | X | X |
| Genotyping^{(j)} | X | | | | | | | | | | | | | |
| Vital sign measurements^{(k)} | X | X | X | X | X | X | X | X | X | X | X | X | X | X |
| 12-Lead ECG^{(l)} | X | | | | X | X | | X | X | X | X | X | X | X |
| Serum chemistry and | X | | X | | X | | | X | X | X | X | X | X | X |
| urinalysis^{(m)} | | | | | | | | | | | | | | |
| Hematology^{(m)} | X | | X | | X | | | X | X | X | X | X | X | X |
| Drug and alcohol screen⁽ⁿ⁾ | X | | | | X | | | | | | | | | |
| Pregnancy test^{(o)} | X | X | | | X | | | | | | X | | X | X |
| 3-Day diet diary^{(p)} | | | X | | X | | | | X | | X | | X | |
| Columbia-Suicide Severity | | | | | X | | | | | | | | | X |
| Rating Scale^{(q)} | | | | | | | | | | | | | | |
| Ocular examination^{(r)} | | | | | X | | | | | | | | | X |
| Study drug administration^{(s)} | | | | | | X | X | X | X | X | X | X | X | |
| PK blood sample | | | | | | X | X | X | X | X | X | X | X | X |
| collection^{(t)} | | | | | | | | | | | | | | |
| PD biomarker sample | | X | X | X | | X | X | X | X | X | X | X | X | X |
| collection^{(u)} | | | | | | | | | | | | | | |
| Adverse events^{(v)} | [symbol] X [symbol] | | | | | | | | | | | | | |
| Prior/concomitant | [symbol] X [symbol] | | | | | | | | | | | | | |
| medications | | | | | | | | | | | | | | |

^{(a)} A safety laboratory visit to collect hematology will occur on Day 4. After the clinic visit on Day 7, patients will have safety laboratory visits every 2 to 3 days (i.e., 2 times per week) between scheduled clinic visits. Hematology tests will be performed at these visits. The frequency of this testing may be increased as per investigator discretion. Home visits may be used for these assessments.
^{(b)}When procedures overlap or occur at the same time point, all blood draws will follow vital signs or ECGs; PK sampling will be timed to occur last and as close to the scheduled time window as possible.
^{(c)}Discharge will occur following completion of the Day 2 assessments.
^{(d)}Genetic testing to confirm diagnosis of PA/MMA if not previously done. Patients that require this testing may have the screening period extended by up to 2 weeks.
^{(e)}Laboratory samples required for eligibility are provided in Section 6.4.2 of the clinical protocol.
^{(f)}Serology testing will include hepatitis B surface antigen, hepatitis C virus antibody, and HIV types 1 and 2 antibodies.
^{(g)}A serum FSH test may be performed for females at Screening to confirm postmenopausal status.
^{(h)}Height and weight will be measured and BMI calculated at Screening only. Only weight will be measured at Check-in and EOS.
⁽ⁱ⁾A full physical examination will be performed at Screening (at minimum, assessment of skin, head, ears, eyes, nose, throat, neck, thyroid, lungs, heart, cardiovascular, abdomen, lymph nodes, and musculoskeletal system/extremities). A brief physical examination will be performed at other stipulated visits (at minimum, assessment of skin, lungs, cardiovascular system, and abdomen [liver and spleen]). Interim physical examinations may be performed at the discretion of the investigator, if necessary, to evaluate AEs or clinical laboratory abnormalities.
^{(j)}Genotyping for CYP2C19.
^{(k)}Vital signs will include systolic and diastolic blood pressure, pulse rate, respiratory rate, and body temperature (temperature once daily with first set of vital signs for the day on inpatient or clinic visit days). Vital signs will be measured after the patient has been resting quietly in the seated position for at least 5 minutes and will be measured at predose (within 90 minutes) and 4 hours (±30 minutes) after the morning dose on Day 1, in the morning on Day 2, and at any time during other clinic visits. A single repeat measurement is permitted at both Screening and Check-in for eligibility determination.
^{(l)}Single 12-lead ECG recordings will be made at stipulated time points after the patient has been in the supine position for at least 5 minutes. A single repeat measurement is permitted at both Screening and Check-in for eligibility determination. Measurements of the following intervals will be reported: RR interval, PR interval, QRS width, QT interval, and QTcF. Assessments will include comments on whether the tracings are normal or abnormal; rhythm; presence of arrhythmia or conduction defects; morphology; any evidence of myocardial infarction; or ST-segment, T-wave, and U-wave abnormalities. Electrocardiogram time points: predose (within 90 minutes) and at 4 hours after the morning dose on Day 1, in the morning on Day 2, and at any time during other clinic visits.
^{(m)}A complete list of assessments is provided in Section 6.4.2 of the clinical protocol. A single repeat measurement is permitted at both Screening and Check-in for eligibility determination.
⁽ⁿ⁾Urine or blood drug and alcohol screen per Section 6.4.2 of the clinical protocol.
^{(o)}Women of childbearing potential only. Urine or serum. The pregnancy test at Check-in should be performed locally to ensure results are available before dosing.
^{(p)}At the screening visit, it must be confirmed that the treatment regimen, including diet, has been stable for at least 30 days. The diet diary will be provided to the patient at the screening visit. For the other visits indicated in this table (starting with the Day −14 run-in visit), the diary must contain data from the 3 consecutive days prior to the clinic visit (at minimum).
^{(q)}Details provided in Section 6.4.4 of the clinical protocol.
^{(r)}Ocular examinations to include best corrected visual acuity, slit lamp testing, and corneal fluorescein staining. Corneal topography may be performed if clinically indicated per assessment of the ophthalmologist. The baseline ocular examination can be done at any time during the run-in period up to Day −1 (Check-in). The EOS ocular examination can be done any time after Day 42 and the EOS visit.
^{(s)}Study drug will be taken orally BID (at least 10 hours apart), starting on Day 1 and will continue for up to 42 days. Study drug must be taken at approximately the same time each day. Planned dose levels and intrapatient dose escalation details are provided in Section 3.3 of the clinical protocol. Patients will be instructed to record the time they take drug each day in a drug diary. Both the drug diary and the bottle of remaining study drug tablets should be brought with the patient to each clinic visit. On outpatient visit days, the morning dose of study drug will be taken in the clinic. Study drug should be administered with room temperature water.
^{(t)}Blood samples for PK analysis will be collected at following time points - Day 1: predose (within 30 minutes prior to the morning dose) and at 1, 2, 4, 6, and at 12 hours after the morning dose (prior to the evening dose); on Day 2: predose (within 30 minutes prior to the morning dose); and on all other scheduled clinic visit days predose (within 30 minutes prior to the morning dose) and a sample between 2 and 4 hours after the morning dose. The EOS sample may be taken at any time during the clinic visit.
^{(u)}Blood (including venous blood gas testing) and urine will be collected for PD biomarker sampling. Venous blood gas testing will be performed locally. All other PD biomarker samples will be analyzed by a central laboratory. PD biomarkers will include whole blood acetyl-CoA, plasma pantothenate, and other exploratory biomarkers.

Blood (including venous blood gas testing) and spot urine will be collected during the clinic visits during the run-in period at Weeks-3, -2, and -1.
Blood (including venous blood gas testing) for PD biomarker sampling will be collected at the following time points after initiation of study drug—Day 1 predose (within 30 minutes prior to the morning dose) and at 1, 2, 4, 6, and at 12 hours after the morning dose (prior to the evening dose); Day 2: predose (within 30 minutes prior to the morning dose); and during all other scheduled clinic visit days: predose (within 30 minutes prior to the morning dose) and a sample between 2 and 4 hours after the morning dose. The EOS sample may be taken at any time during the clinic visit.
Spot urine samples for PD biomarkers will be collected at the following time points after the initiation of study drug: on Day 1 within 2 hours prior to the morning dose and a spot sample between 4 and 6 hours after the morning dose, on Day 2 within 2 hours before the morning dose, and during all other scheduled clinic visit days within 2 hours prior to the morning dose and between 2 and 4 hours after the morning dose. The EOS sample may be taken at any time during the clinic visit.
Adverse events will be assessed from the time of signing of informed consent until EOS and must be followed until they are resolved, stable, or judged by the investigator to be not clinically significant.

### 4.3 Study Population

Approximately 64 healthy male and female subjects will be enrolled in the SAD portion of the study, approximately 48 healthy male and female subjects will be enrolled in the MAD portion of the study, and up to 16 male and female patients (up to 8 patients each with PA or MMA) will be enrolled in the PA/MMA portion of the study.

### Inclusion Criteria

Each healthy subject or patient with PA/MMA must meet all of the following criteria to be enrolled in this study:
1. The healthy subject or patient with PA/MMA is male or female 18 to 55 years of age, inclusive, at Screening.
2. The healthy subject has a BMI 18 to 32 kg/m², inclusive, at Screening. Every attempt must be made to enroll at least 2 healthy subjects weighing <60 kg in each SAD cohort. Patients with PA/MMA are not required to meet specific BMI requirements.
3. Female healthy subjects or patients with PA/MMA must fulfill the following criteria:
   a. If of childbearing potential, must use a highly effective method (<1% failure rate) of birth control (e.g., oral hormonal contraception, intrauterine device, azoospermic partner, sexual abstinence) during participation in the study until at least 2 months after the last dose of study drug, or
   b. Is surgically sterile (i.e., hysterectomy, bilateral tubal ligation or bilateral oophorectomy) at least 6 months prior to the first dose of study drug, or
   c. Is postmenopausal (defined as amenorrhea 12 consecutive months and documented plasma follicle-stimulating hormone level >40 IU/mL).
4. Female healthy subjects or patients with PA/MMA must have a negative pregnancy test at Screening and Check-in.
5. Male healthy subjects or patients with PA/MMA must agree to use one of the following contraceptive methods: double-barrier contraception during the study until at least 90 days after the last dose of study drug (even with a partner of non-childbearing potential) or practice sexual abstinence.
6. Male healthy subjects or patients with PA/MMA must not donate sperm during the study and for 90 days after receiving the last dose of study drug.
7. Healthy subjects in the MAD cohort must have an absolute neutrophil count ≥2500×10⁹/L. Patients with PA or MMA must have an absolute neutrophil count ≥1500×10⁹/L.
8. The healthy subject or patient with PA/MMA agrees to comply with all protocol requirements.
9. The healthy subject is able to provide written informed consent. For patients with PA/MMA, the patient or the patient's legally acceptable representative is able to provide written informed consent.

### Healthy Subjects Only:

10. The healthy subject has no clinically significant history or presence of ECG findings as judged by the investigator at Screening and Check-in, including each criterion as follows:
   a. Normal sinus rhythm (HR between 45 bpm and 100 bpm inclusive; values slightly outside the normal range may be acceptable if they are considered not clinically significant by the investigator after discussion with the sponsor);
   b. QTcF≤450 msec for male and ≤470 msec for female;
   c. QRS interval ≤120 msec; confirmed by manual over-read if >120 msec;
   d. PR interval ≥120 and ≤220 msec.
11. The healthy subject is considered by the investigator to be in good general health as determined by medical history, clinical laboratory test results, vital sign measurements,
12-lead ECG results, and physical examination findings at Screening. A single repeat of abnormal clinical laboratory tests, vital sign measurements, and ECG results is allowed at both Screening and Check-in to determine eligibility.
   Patients with PA or MMA Only
12. For PA patients, the patient has confirmed PA diagnosed by all of the following criteria (may be based on historical records):
   a. Elevated plasma/DBS/urine 2-methylcitrate and/or 3-hydroxypropionate;
   b. Elevated plasma/serum/DBS propionyl carnitine; and
   c. PCCA/PCCB mutations confirmed by molecular genetic testing.
13. For MMA patients, the patient has confirmed MMA diagnosed by all of the following criteria (may be based on historical records):
   a. MUT deficiency (mut0 or mut-);
   b. Elevated plasma, serum, DBS, or urine MMA levels;
   c. Presence of normal serum or plasma vitamin B12 and plasma homocysteine levels; and
   d. Confirmed by molecular genetic testing.
14. A patient with MMA currently has elevated plasma MMA levels.
15. A patient with PA/MMA or patient's legally acceptable representative is willing and able to provide access to medical records charting for the last 6 to 12 months of care prior to study initiation.
16. A patient with PA/MMA is currently appropriately managed on consistent disease management, which may include low-protein/high-energy diet and carnitine supplements. The treatment regimen (including diet) must be stable for at least 30 days prior to entering the study. Once study drug treatment has started, must be willing to maintain all aspects of the treatment regimen and diet unchanged.

### Exclusion Criteria

Healthy subjects or patients with PA/MMA meeting any of the following criteria will be excluded from the study:
1. The healthy subject or patient with PA/MMA has used prescription drugs (contraceptive medications are allowed) within 4 weeks before the first dose of study drug and/or over-the-counter medication including routine vitamins within 7 days of the first dose of study drug and is unable or unwilling to refrain from such use through the EOS visit. For the COVID-19 vaccine, the most recent vaccine dose (or booster) must be at least 14 days prior to first dose of study drug. Patients with PA/MMA who are on stable doses of supplements and/or medication for their condition or for other reasons may be enrolled on a case-by-case basis subject to a review of all concomitant medication/supplements by the sponsor to rule out potential drug-drug interactions.
2. The heathy subject or patient with PA/MMA is unable or unwilling to refrain from wearing contact lenses during participation in the study.
3. The healthy subject or patient with PA/MMA has a history of dry eye or eye surgery, including radial keratotomy and LASIK surgery.
4. The healthy subject or patient with PA/MMA has abnormal laboratory test results. A single repeat of abnormal results is allowed both at Screening and Check-in to determine eligibility. Slight excursions above or below the reference range may be allowed if they are not considered clinically significant by the investigator after discussion with sponsor. Abnormalities considered by the investigator to be the result of underlying organic acidemias for the PA/MMA cohort are allowed. Excursions in the PA/MMA cohort that are not PA/MMA related may be allowed if they are considered not clinically significant by the investigator after discussion with sponsor; however, test results within the following ranges will exclude a patient from participation in the study:
   a ALT or AST≥2×ULN;
   b Total bilirubin ≥2×ULN; or
   c INR≥1.4.
5. The healthy subject has a baseline eGFR<90 mL/minute calculated using the CKD-EPI formula. The patient with PA/MMA has a baseline eGFR<45 mL/min calculated using the CKD-EPI formula.
6. The healthy subject or patient with PA/MMA has a positive test result for hepatitis B surface antigen, hepatitis C virus antibody, or HIV types 1 or 2 antibodies at Screening.
7. The healthy subject or patient with PA/MMA fails SARS-CoV-2 screening requirements, as per site practice.
8. The female healthy subject or patient with PA/MMA is pregnant, trying to become pregnant, or is lactating.
9. The healthy subject or patient with PA/MMA has consumed grapefruit or grapefruit juice, Seville orange or Seville orange-containing products (e.g., marmalade), or caffeine- or xanthine-containing products within 48 hours before study drug dosing or throughout the study. Please note caffeine is allowed for treatment of AEs (e.g., post lumbar puncture headache) at the discretion of the investigator.
10. The healthy subject is a smoker or has used nicotine or nicotine-containing products (e.g., snuff, nicotine patch, nicotine chewing gum, mock cigarettes, or inhalers) within 6 months before study drug dosing.
11. The healthy subject or patient with PA/MMA has a history of alcohol abuse or drug addiction within 12 months before study drug dosing.
12. The healthy subject has a positive test result for drugs of abuse, alcohol, or cotinine (indicating active current smoking) at Screening, before study drug dosing, or throughout the study.
13. The healthy subject or patient with PA/MMA is involved in strenuous activity or contact sports within 24 hours before study drug dosing or throughout the study.
14. The healthy subject or patient with PA/MMA has donated blood or blood products >450 mL within 30 days before study drug dosing.
15. The healthy subject or patient with PA/MMA has a history of relevant drug and/or food allergies (i.e., allergy to any study drug or excipients or any significant food allergy that could preclude a standard diet in the clinical unit).
16. The healthy subject or patient with PA/MMA has received a study drug in another investigational study within 30 days of dosing.
17. The healthy subject or patient with PA has undergone prior liver and/or kidney transplant. Prior liver and/or kidney transplant is allowed for patients with MMA.
18. The healthy subject or patient with PA/MMA is not suitable for entry into the study in the opinion of the investigator.
   Patients with PA or MMA Only:
19. The patient with PA/MMA has Grade 3 or 4 heart failure according to the Modified Ross Heart Failure Classification for Children or the New York Heart Association.
20. The patient with PA/MMA has a QTcF of >450 msec on ECG at Screening or Check-in.
21. The patient with PA/MMA has had a recent infection requiring systemic antibiotics within 4 weeks of Baseline or active infection that should preclude the patient's participation in the study, in the opinion of the investigator.
22. The patient with PA/MMA has a positive test result for drugs of abuse or alcohol at Screening or Check-in.
23. The patient with PA/MMA has been exposed to gene therapy for PA or MMA at any time prior to study entry.

### 4.4 Study Treatments

All healthy subjects or patients with PA/MMA will receive the study treatments as described herein and according to the SOEs (Table 6, Table 7, Table 8, and Table 9). Additional instructions for dosing, fasting periods, and nonfasting periods can be found in the SOEs.

Listerine® strips will be given immediately before and after blinded drug administration. Healthy subjects will be instructed to place 2 Listerine strips on their tongue until completely dissolved and then take study drug followed by 2 additional Listerine strips. Additional Listerine strips can be given if needed to ensure no taste can be detected while taking the study drug. Listerine strips will only be given to blinded cohorts. Healthy subjects in the SAD FE Cohort and patients in the PA/MMA Cohort will not be given the Listerine strips.

### Dose Escalation and Safety Review Committee

### Single-Ascending Dose and Multiple-Ascending Dose Cohorts in Healthy Subjects

This study is designed such that dose escalation to the next planned dose level in the SAD/MAD cohorts is allowed only after a blinded review of data from at least 6 subjects in the current dose level by the SRC. The SRC will review a minimum of 48 hours of safety data and 24 hours of PK data; safety data will include clinical laboratory results, vital signs, ECGs, and AEs. Dosing in the next higher dose cohort will be permitted only after the review of safety data suggests it is safe to do so and must not occur until written/e-mail confirmation is provided by the SRC. Based on the review of safety and PK data, the SRC may choose to repeat a dose level, administer a dose less than the previous dose, or administer a dose that varies in another way from the next planned dose. Before implementing any change, the IRB will be notified and provided with the rationale.

The SRC for the SAD/MAD portions of the study will include the sponsor clinical scientist(s), PK specialist(s), the PPD medical monitor, and the principal investigator at the PPD clinical research unit. Additional study team members (nonvoting) may attend SRC meetings for awareness or to assist with organizing and recording SRC decisions.

### Propionic Acidemia and Methylmalonic Acidemia Cohort

The SRC for the PA/MMA cohort will be reconstituted with new members with that include experts in the PA/MMA patient population and may also include outside experts to provide independent evaluation of subject safety.

The SRC will meet periodically; in addition, an SRC meeting will be triggered if 2 or more patients develop neutropenia at a specific dose, or if severe neutropenia occurs in a PA/MMA patient.

The operating rules of the SRC will be outlined in a separate charter and will include aspects such as roles and responsibilities, designation of voting members, timing of reviews/responses, scheduled and ad hoc meetings, and minimum attendance requirements for a quorum.

### Dose-Limiting Toxicities (DLTs) for Healthy Subjects

A DLT may be defined as any of the following:
A CTCAE (NCI 2017) Grade 3 or higher TEAE experienced by 2 or more subjects in a cohort, who, after unblinded safety review, are confirmed to have received study drug.
An SAE or any other medically important TEAE that impacts subject safety experienced by 1 or more subjects in a cohort, who after unblinded safety review, are confirmed to have received study drug.
A DLT may be considered for a subject who withdraws because of an intolerable TEAE.

Dose Escalation Stopping Criteria for Healthy Subjects Dose escalation in the SAD and MAD cohorts will be suspended if any of the following occur:
Any preclinical or clinical events that, in the opinion of the SRC, contraindicate further dosing of additional subjects with study drug.
In the opinion of the SRC, data from the previous dose cohort indicating safety concerns for the next cohort to be dosed at a higher level, such as unanticipated responses (e.g., clinically significant changes in clinical laboratory data, ECGs, cardiac telemetry, vital signs, or physical examinations).
Two or more subjects in a dose cohort experience a CTCAE Grade 3 or higher TEAE that, in the opinion of the SRC, warrants suspension of dose escalation.
Two or more subjects receiving study drug have >3 times the ULN of either ALT or AST or >2 times the ULN for bilirubin or alkaline phosphatase where no other reason can be found to explain the increases.
Two or more subjects receiving study drug experience a CTCAE Grade 2 or higher gastrointestinal bleed or increase in serum creatinine.

Dose escalation may also be suspended if, in the opinion of the investigator, sponsor, PPD medical monitor, or SRC, any other significant safety or tolerability issues are identified in the comprehensive review of available data that warrant further evaluation before additional subjects are dosed. This may include emerging nonclinical data, clinically relevant AEs, or relevant data from other sources indicating safety concerns even if the event(s) per se does not meet the protocol-specified definition of a DLT.

### Stopping Criteria for Multidose Cohorts

### Multiple-Ascending Dose Cohorts (Healthy Subjects)

Dosing in the MAD cohorts will be stopped for the entire dose cohort if any of the following occur:
Any events that, in the opinion of the investigator/PPD medical monitor (after discussion with the sponsor), contraindicate further dosing with study drug.
Two or more healthy subjects in a dose cohort experience a CTCAE Grade 3 or higher TEAE that, in the opinion of the investigator/PPD medical monitor (after discussion with the sponsor), warrants stopping dosing of the entire cohort.
Two or more healthy subjects receiving study drug have >3 times the ULN of either ALT or AST or >2 times the ULN for bilirubin or alkaline phosphatase where no other reason can be found to explain the increases.
Two or more healthy subjects receiving study drug experience a CTCAE Grade 2 or higher gastrointestinal bleed or increase in serum creatinine.
Two or more healthy subjects on active drug in a dose cohort experience an ANC of less than the lower limit of normal confirmed by a repeat measurement.

At the discretion of the investigator/PPD medical monitor (after discussion with the sponsor), dosing in any given subject may be stopped at any time for any TEAE and/or if it is deemed to be in the best interest of the subject.

### Propionic Acidemia and Methylmalonic Acidemia Cohort

Dosing in the PA/MMA cohort will be stopped for the entire dose cohort if any of the following occur:
Any events that, in the opinion of the investigator/PPD medical monitor (after discussion with the sponsor), contraindicate further dosing with study drug.
Two or more patients with PA/MMA in the cohort experience a CTCAE Grade 3 or higher TEAE that, in the opinion of the investigator/PPD medical monitor (after discussion with the sponsor), warrants stopping dosing of the entire cohort.
Two or more patients with PA/MMA receiving study drug experience a CTCAE Grade 2 or higher gastrointestinal bleed or increase in serum creatinine.

### Individual Stopping Criteria and Dose Adjustments (Propionic Acidemia and Methylmalonic Acidemia Cohort)

Any PA/MMA patients that are classified as “poor metabolizers” based on CYP2C19 genotyping will only receive the morning dose.

Absolute neutrophil values will be monitored throughout the study. For cases of mild, moderate, or severe neutropenia to be confirmed, there must be 2 consecutive ANC counts a maximum of 2 days apart that are both less than a specific value as indicated below. If, at any time, a patient with PA/MMA has a confirmed ANC level below 1.5×10⁹/L, the investigator is to use the following guidance:
Mild Neutropenia: If the confirmed ANC is <1.5×10⁹/L but ≥1.0×10⁹/L, continue treatment without interruption, but monitor the ANC every 2 days until resolution.
Moderate Neutropenia: If the confirmed ANC is <1.0×10⁹/L but ≥0.5×10⁹/L, interrupt treatment and monitor ANC every 2 days until resolution. Treatment can be re-initiated once the event is resolved. If the ANC is still <1.5×10⁹/L after 14 days, withdraw the patient from the study and monitor until resolution of the event.
Severe Neutropenia: If a single ANC is <0.5×10⁹/L, immediately interrupt treatment without waiting for confirmation of the ANC and obtain a second measurement as soon as possible, but no later than the next day. If the second ANC is still <0.5×10⁹/L after 2 days, permanently withdraw the patient from the study and follow the local standard of care procedures for the management of severe neutropenia.

At the discretion of the investigator/PPD medical monitor (after discussion with the sponsor), dosing in any given patient with PA/MMA may be stopped at any time for any TEAE and/or if it is deemed to be in the best interest of the patient.

Patients in the PA/MMA cohort may have the planned doses adjusted in the event of neutropenia, as indicated in FIG. 15.

### 4.5 Study Assessments and Endpoints

### Pharmacokinetic Assessments and Endpoints

The timing and frequency of PK sample collection are listed in the SOEs (Table 6, Table 7, Table 8, and Table 9).

Allowable windows for PK samples for healthy subjects are provided in the laboratory manual. Allowable windows for PK and PD samples for PA/MMA patients are provided in Table 10.

TABLE 10
Windows for Pharmacokinetic and Pharmacodynamic Samples
for Propionic Acidemia and Methylmalonic Acidemia Cohort

| | |
|---|---|
| Sample Time Point | Allowable Window |

Blood

| | |
|---|---|
| Day 1 predose | −30 minutes to 0 hour |
| Day 1 postdose: 1, 2, and 4 hours | ±10 minutes |
| Day 1 postdose: 6 and 12 hours | ±30 minutes |
| Day 2 predose | −30 minutes to 0 hour |
| Days 7 to 42 | Predose: −30 minutes to 0 hour |
| | Postdose: Between 2 and 4 hours |
| End of study | Any time during clinic visit |

Spot Urine

| | |
|---|---|
| Day 1 predose | −2 hours to 0 hour |
| Day 1 postdose | Between 4 and 6 hours |
| Day 2 predose | −2 hours to 0 hour |
| Days 7 to 42 | Predose: −30 minutes to 0 hour |
| | Postdose: Between 2 and 4 hours |
| End of study | Any time during clinic visit |

Pharmacokinetic samples will be analyzed using validated liquid chromatography coupled with tandem mass spectrometry assays for Compound (I) in human plasma, urine, and CSF.

### Pharmacodynamic Assessments and Endpoints

Pharmacodynamic biomarker assessments will include whole blood acetyl-CoA, plasma pantothenate, and other exploratory PD biomarkers.

The allowable windows for PD biomarkers for the PA/MMA cohort are provided in Table 10.

Pharmacodynamic biomarker samples will be analyzed using a validated assay for the PD assessments.

### Cardiodynamic Electrocardiogram Assessments and Endpoints

Cardiodynamic assessments are applicable to the healthy subject cohorts only.

### Safety Assessments and Endpoints

The timing and frequency of all safety assessments are listed in the SOEs (Table 6, Table 7, Table 8, and Table 9).

Safety and tolerability endpoints will include monitoring and recording of AEs, clinical laboratory test results (hematology, serum chemistry, and urinalysis), vital sign measurements, 12-lead ECG results, and physical examination findings. The PA/MMA cohort will also be screened for suicidal ideation using the C-SSRS.

For all safety assessments, the investigator will determine whether results are clinically significant, which is defined as any variation in a result that has medical relevance and may result in an alteration in medical care (e.g., active observation, diagnostic measures, or therapeutic measures). For clinical laboratory test results and vital sign measurements, slight excursions outside normal limits may be permissible, if deemed not clinically significant by the investigator/PPD medical monitor after discussion with sponsor. If clinical significance is noted, the result and reason for significance will be documented and an AE reported on the AE page of the healthy subject or patient with PA/MMA eCRF. The investigator will monitor the healthy subject or patient with PA/MMA until the result has reached the reference range or the result at Screening, or until the investigator determines that follow-up is no longer medically necessary.

The following clinical laboratory assessments will be performed at the time points indicated in the SOE (Table 6, Table 7, Table 8, and Table 9):
Hematology White blood cell count, red blood cell count, hemoglobin, hematocrit, platelets, white blood cell differential (percentage and absolute): neutrophils, lymphocytes, monocytes, eosinophils, and basophils. For patients in the PA/MMA cohort, in addition to the parameters listed above, reticulocytes will be analyzed during scheduled clinic visits. Bands will be analyzed during scheduled clinic visits and during the safety laboratory visits.
Serum Chemistry Albumin, alkaline phosphatase, ALT, AST, amylase, bilirubin (total and direct), blood urea nitrogen, calcium, carbon dioxide, chloride, cholesterol (total, high-density lipoprotein, low-density lipoprotein), creatine kinase, creatinine, gamma glutamyltransferase, globulin, glucose, lactic dehydrogenase, phosphate, potassium, sodium, total protein, triglycerides, uric acid
Urinalysis Urinalysis dipstick will be performed at the local site. Appearance, bilirubin, color, creatinine, glucose, ketones, leukocytes, nitrites, occult blood, pH, protein, specific gravity, turbidity, and urobilinogen Reflex microscopy (performed if dipstick is positive for protein or the blood value is 1+ or greater; performed at a central laboratory) includes bacteria, casts, crystals, epithelial cells, mucus, red blood cells, white blood cells, and yeast
Serology Hepatitis B surface antigen, hepatitis C virus antibody, and HIV antibody types 1 and 2 SARS-CoV-2 (as per site practice)
Other analyses Healthy subjects only:
   Urine or blood drug screen (including alcohol and cotinine)
   Genotyping for CYP2C19 and CYP2D6
   PA/MMA patients only:
   Urine or blood drug screen (including alcohol), performed locally
   Genotyping for CYP2C19
   Laboratory samples required for eligibility: Coagulation (prothrombin time, activated partial thromboplastin time, international normalized ratio), Vitamin B12, plasma acid pathways, acylcarnitine plasma; genetic testing for PA/MMA mutations if not previously done
   Female healthy subjects and patients with PA/MMA: FSH, pregnancy test (human chorionic gonadotropin); serum or urine

### Example 5: Safety and Tolerability of Single- and Multiple-Doses of Compound (I)

Table 11 and Table 12 show results of the single- and multiple-ascending dose studies described in Example 4. As shown in the tables, no serious adverse events (SAEs) were observed with treatment with Compound (I). Mild treatment-related treatment emergent adverse events (TEAEs) included headache, abdominal pain, and nausea (5%, 1.7%, and 1.7%, respectively, of all Compound (I) treated subjects). Asymptomatic neutropenia was observed in 3 subjects of 22 with repeat dosing of Compound (I). All returned to within normal limits within a few days without any sequelae upon cessation of therapy. There was no apparent association of neutropenia with higher Compound (I) exposures. ANC nadirs for subjects who experienced neutropenia in the multiple-ascending dose (MAD) cohort were 740/μL and 1320/μL with 50 mg of Compound (I) twice daily.

TABLE 11
Safety and tolerability of single-ascending doses of Compound (I).

| | | | | | 60 mg | | | | |
|---|---|---|---|---|---|---|---|---|---|
| | | | | | (N = 8) | | | | |
| | | | | | Food | | | | |
| | Placebo | 3 mg | 10 mg | 30 mg | Effect | | 100 mg | 120 mg | |
| | (N = 10) | (N = 6) | (N = 6) | (N = 6) | Study | | (N = 6) | (N = 6) | |
| Subjects | 0 (0%) | 0 (0%) | 0 (0%) | 0 (0%) | 0 | (0%) | 0 (0%) | 0 | (0%) |
| with SAEs | | | | | | | | | |
| Subjects | 0 (0%) | 0 (0%) | 0 (0%) | 0 (0%) | 2 | (25%)* | 0 (0%) | 1 | (17%) |
| with | | | | | | | | | |
| TEAEs | | | | | | | | | |

*Treatment-related TEAEs in the SAD cohorts included headache abdominal pain, and nausea

TABLE 12
Safety and tolerability of multiple-
ascending doses of Compound (I).

| | | 30 mg | 60 mg | | 70 mg | 50 mg | |
|---|---|---|---|---|---|---|---|
| | Placebo | QD | QD | | QD | Q12H* | |
| | (N = 7) | (N = 6) | (N = 6) | | (N = 4) | (N = 6) | |
| Subjects | 0 (0%) | 0 (0%) | 0 | (0%) | 0 (0%) | 0 | (0%) |
| with | | | | | | | |
| SAEs | | | | | | | |
| Subjects | 0 (0%) | 0 (0%) | 1 | (17%) | 0 (0%) | 2 | (33%) |
| with | | | | | | | |
| TEAEs | | | | | | | |

*Treatment-related TEAEs in the MAD cohorts included neutropenia

As shown in Table 13, Compound (I) was detected in the cerebrospinal fluid (CSF) in all subjects dosed 70 mg QD at steady state. Geometric mean ratio of the partition coefficient (CSF/plasma) was 0.0241, showing approximately 2.4% of systemic plasma concentrations penetrated the CSF.

TABLE 13
Compound (I) detected in CSF

| | CSF | Partition Coefficient |
|---|---|---|
| Statistic | Concentration | (CSF/Plasma) |
| GM | 8.33 | 0.0241 |
| (% GCV) | (20.6) | (26.7) |

Abbreviations:
GM = geometric mean;
% GCV = percent geometric coefficient of variation;
N = number of subjects;
QD = once daily
Note:
CSF samples were collected ~2 hours post-dose. A plasma PK sample was also collected immediately after the lumbar puncture.

### Example 6: Pharmacokinetics (PK) and Pharmacodynamics (PD) of Compound (I)

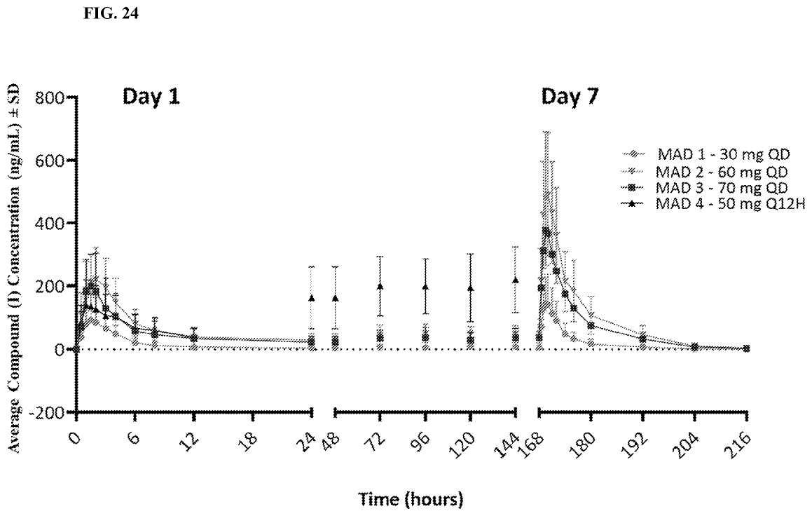

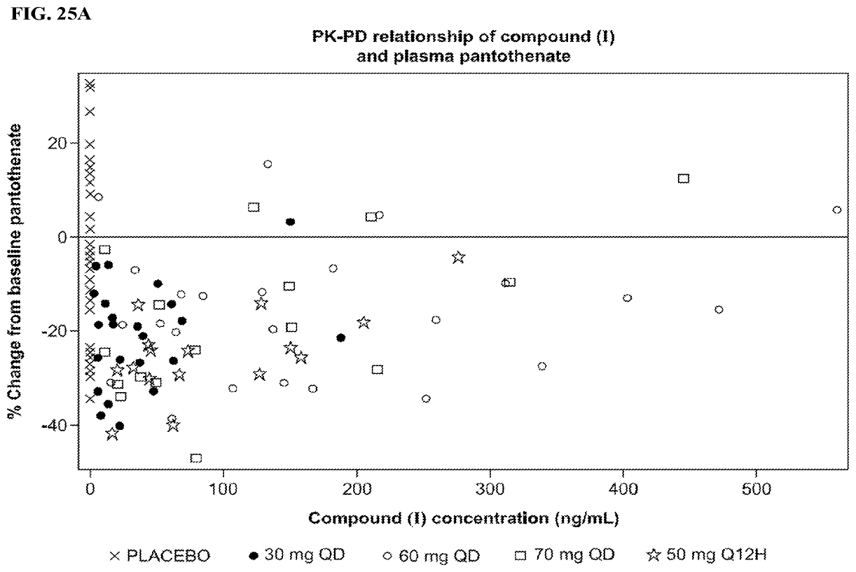

FIG. 24 shows the pharmacokinetics upon repeated dosing of Compound (I) as described in Example 4, and Table 14 summarizes relevant pharmacokinetic parameters. Compound (T) was readily absorbed after oral administration with a Tₘₐₓ of ˜1-2 hours and t_{1/2} averaged 6-8 hours. The increase in exposure was more than dose-proportional as the total daily dose increased from 30 mg to 100 mg. The presence of food delayed absorption of Compound (I) by ˜2 hours and showed a modest increase in plasma exposure (˜1.3 fold), which was not considered clinically relevant. Urinary excretion of Compound (I) was negligible (<% of the administered dose).

TABLE 14
Pharmacokinetics of Compound (I)

| | | 30 mg QD | | 60 mg QD | | 70 mg QD | | 50 mg Q12H | |
|---|---|---|---|---|---|---|---|---|---|
| | | (N = 6) | | (N = 6) | | (N = 4) | | (N = 6) | |
| Parameter | Statistic | Day 1 | Day 7 | Day 1 | Day 7 | Day 1 | Day 7 | Day 1 | Day 7 |
| AUCₜₐᵤ^{a} | AM | 476 | 913 | 1670 | 4050 | 1320 | 2950 | 925 | 3330^{b} |
| (ng*h/mL) | (SD) | (464) | (569) | (884) | (1850) | (1010) | (1430) | (558) | |
| Cₘₐₓ | AM | 98.3 | 158 | 240 | 505 | 205 | 400 | 155 | 340^{b} |
| (ng/mL) | (SD) | (82.7) | (88.2) | (94.4) | (203) | (110) | (106) | (74.5) | |
| Tₘₐₓ (h) | Median | 1.5 | 1.5 | 1.75 | 2.01 | 1.50 | 1.50 | 1.50 | N/A |
| t_{1/2} (h) | AM | ND | 7.55 | ND | 6.70 | ND | 6.83 | ND | N/A |
| | (SD) | | (3.16) | | (1.49) | | (1.96) | | |

Abbreviations:
AM = arithmetic mean;
SD = standard deviation;
N = number of subjects;
QD = once daily;
Q12H = every 12 hours;
N/A—not applicable
Note:
Subjects were not dosed on Day 7 in the 50 mg Q12H cohort; therefore, PK parameters were predicted using population PK modeling.
^{a}AUCₜₐᵤ represents AUC₀₋₂₄ for 30, 60, and 70 mg QD Compound (I) treatments; AUCₜₐᵤ represents AUC₀₋₁₂ for 50 mg Q12H Compound (I) treatment.
^{b}50 mg Q12H Day 7 PK parameters predicted using population PK modeling performed by Certara.

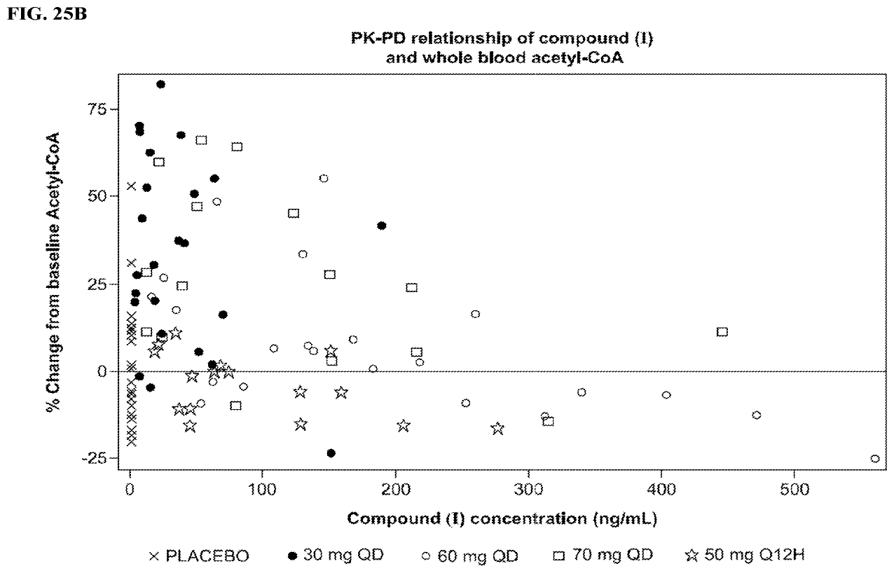

FIGS. 25A-25B shows the PK-PD relationship of Compound (I) and plasma pantothenate (FIG. 25A) or whole blood acetyl-CoA (FIG. 25B). In MAD cohorts at steady-state, decreases in the PanK substrate, pantothenate, were observed, concurrent with increases in the PanK product, acetyl-CoA, in whole blood. Increases in whole blood acetyl-CoA were observed up to plasma Compound (I) concentrations of ˜200 mg/mL. Decreases in plasma pantothenate were observed across Compound (I) exposures tested. Changes in pantothenate and acetyl-CoA demonstrated target engagement in a peripheral compartment in healthy adult subjects.

Overall, Compound (I) was generally well-tolerated in 60 healthy adult subjects in a Phase 1 study. No SAEs were observed. The most common mild treatment-related TEAEs were headache, abdominal pain, and nausea. Asymptomatic neutropenia was observed in 3 of 22 subjects with repeat Compound (I) dosing. All returned to within normal limits within a few days without any sequelae upon cessation of therapy. Compound (I) was orally bioavailable and was detected in CSF, indicating Compound (I) crossed the blood brain barrier. Compound (I) increased whole blood acetyl-CoA levels and decreased plasma pantothenate levels in healthy adult subjects, demonstrating target engagement and proof of mechanism. Based on these data, Compound (I) will be further studied in patients with propionic acidemia and methylmalonic acidemia.

### Example 7. A Randomized, Double-Blind, Placebo-Controlled Study to Evaluate the Efficacy, Safety, and Tolerability of Compound (I) in Patients with Pantothenate Kinase-Associated Neurodegeneration (PKAN)

### 7.1 Study Objectives

Efficacy Objective: To evaluate the efficacy of Compound (I) in patients with pantothenate kinase-associated neurodegeneration (PKAN)

Safety Objective: To assess the safety and tolerability of Compound (I) in patients with PKAN

### Other Objectives:

To determine the pharmacokinetics (PK) of Compound (I) following multiple doses in patients with PKAN
To explore potential biomarkers of disease and their responses to Compound (I) in patients with PKAN

### 7.2 Study Design and Methodology

This is a randomized, double-blind, placebo-controlled, multi-center, 2-arm study evaluating 72 weeks of treatment with Compound (I) or placebo in patients aged 6 to 65 years with PKAN who were diagnosed with dystonia before the age of 10 years. An independent Data Monitoring Committee (DMC) will monitor the safety of study participants.

The study seeks to enroll approximately 84 participants. After a 4-week screening period, patients meeting all eligibility criteria will be enrolled in the study and randomized in a 2:1 ratio to receive either Compound (I) or placebo. Randomization will be stratified by age at dystonia onset (<6 or 6-10 years) and disease duration (<10 or ≥10 years). After randomization, participants will enter a 12-week run-in period for baseline assessment of disease severity, followed by 72 weeks of double-blind study drug administration. Participants who complete the study will be invited to participate in a separate open-label extension study.

Study drug (Compound (I) or matching placebo) will be administered twice daily (BID), with step-up dosing: a lower dose will be administered during the first 2 weeks, then (as tolerated) titration of study drug to the full treatment dose will occur. The initial dose will be determined from the totality of available data at that time, including nonclinical data and any available data from the first-in-human Study CoA-101 of Compound (I) (see Example 4).

In the first stage, patients who are aged ≥16 years and weigh ≥55 kg will be enrolled and treated with this dose. The DMC will review initial tolerability and safety when 10 participants total have been treated for ≥30 days. Population PK analysis will examine the relationship between body weight and exposure, and modeling and simulations will determine appropriate dose adjustments for patients who weigh ≥35 kg.

In the second stage, patients who are aged ≥12 years and weigh ≥35 kg will be enrolled and treated. The DMC will review tolerability and safety when 6 participants total aged 12 to 15 years have been treated for ≥30 days. Further modeling and simulations based on all available PK data will determine appropriate dose adjustments for patients weighing ≥18 kg.

In the third stage, after juvenile toxicology data are available, patients who are aged ≥6 years and weigh ≥18 kg will be enrolled and treated with the appropriate dose adjustments.

Clinical laboratory testing (hematology, chemistry) will occur at the visits shown in the Schedule of Events. Dose modification for neutropenia will occur as follows:
Mild neutropenia: If confirmed absolute neutrophil count (ANC) is ≥1.0×10⁹/L and <1.5×10⁹/L, then treatment may be continued without interruption; monitor ANC every 2 days until resolution.
Moderate neutropenia: If confirmed ANC is ≥0.5×10⁹/L and <1.0×10⁹/L, then interrupt treatment and monitor ANC every 2 days until resolution.
The participant may restart treatment at the same dose within 14 days after the event is resolved.
If ANC is still <1.5×10⁹/L after 14 days, then withdraw participant from dosing and monitor until resolution of the event.
Severe neutropenia: If confirmed ANC is <0.5×10⁹/L, then interrupt treatment and obtain a second measurement the following day.
If ANC is still <1.5×10⁹/L within 3 days, then permanently withdraw the participant from dosing and follow standard of care procedures for the management of severe neutropenia.
If ANC is ≥1.5×10⁹/L within 3 days, then then the participant may restart treatment at a lower dose.

### 7.3 Diagnosis and Criteria for Inclusion and Exclusion:

### Inclusion Criteria:

A patient must meet all of the following criteria to be eligible for this study:
1. The patient has a documented diagnosis of PKAN as indicated by a confirmed mutation in the pantothenate kinase 2 (PANK2) gene (may be based on historical records).
2. The patient is aged 6 to 65 years at the time of informed consent.
3. The patient or parent/legal guardian (as appropriate) is willing and able to provide a signed informed consent, and where required, the patient is willing to provide assent.
4. The patient was diagnosed with dystonia before the age of 10 years.
5. The patient is currently appropriately managed on consistent disease management. The treatment regimen must be stable for at least 30 days before entering the study. Once study drug treatment has started, the patient must be willing to maintain all aspects of the treatment regimen unchanged.
6. If sexually active with male partners, the female patient of childbearing potential agrees to use a medically acceptable method of contraception for the duration of the study and for at least 2 months after the last dose of study drug. Female patients are considered of childbearing potential if they are postmenarchal, have not been surgically sterile for at least 6 weeks (ie, total hysterectomy, bilateral salpingo-oophorectomy, tubal ligation) and are premenopausal (menopause is defined as cessation of menstruation for at least 1 year). Acceptable methods of contraception include:
   a. the simultaneous use of stable combined (estrogen and progestogen containing) or progestogen-only hormonal contraception (eg, oral, transdermal, intravaginal) associated with inhibition of ovulation, in conjunction with a double-barrier method (eg, condom with spermicide, diaphragm with spermicide); or
   b. the use of an intrauterine device (IUD) or intrauterine hormone-releasing system (IUS) in place for at least 3 months; or
   c. vasectomized partner (vasectomy at least 3 months prior to screening); or
   d. sexual abstinence
6. If sexually active with female partners, the sexually mature, nonsterile male patient agrees to use a medically acceptable method of contraception for the duration of the study and for at least 90 days after the last dose of study drug. Male patients are considered surgically sterile if they have undergone bilateral orchiectomy or vasectomy at least 3 months prior to screening. Acceptable methods of contraception include:
   a. the female partner's simultaneous use of stable combined (estrogen and progestogen containing) or progestogen-only hormonal contraception (eg, oral, transdermal, intravaginal) associated with inhibition of ovulation, in conjunction with a double-barrier method (eg, condom with spermicide, diaphragm with spermicide); or
   b. the female partner's use of an IUD or IUS in place for at least 3 months; or
   c. the female partner is surgically sterile for at least 6 weeks or is at least 1 year postmenopausal.
7. The male patient agrees not to donate sperm for at least 90 days after the last dose of study drug.

### Exclusion Criteria:

Patients who meet any of the following criteria will be excluded from this study:
1. The patient is unable or unwilling to remain on their pre-study dose(s) of allowed concomitant PKAN maintenance medications and therapies (including deep brain stimulation [DBS] settings) during the study.
2. The patient has required regular or intermittent invasive ventilatory support to maintain vital signs within 24 weeks prior to randomization.
3. The patient has received a COVID-19 vaccine dose (or booster) within 14 days before the first dose of study drug.
4. The patient is treated with a strong or moderate CYP3A4 inhibitor, or a strong CYP2C19 inhibitor, within 30 days before the first dose of study drug.
5. The patient has been (or is currently) enrolled in a clinical trial involving a study drug or non-indicated use of a drug or device within 30 days prior to randomization.
6. The patient has a positive serologic test for hepatitis B virus surface antigen (HBsAg), hepatitis C virus (HCV) antibody, or human immunodeficiency virus (HIV) at screening.
7. The patient has a history of metastasized or ongoing malignancy, regardless of whether or not it has been or is being treated.
8. The patient has a serious, unstable medical or psychiatric condition not related to PKAN that, in the opinion of the Investigator, could interfere with the patient's ability to participate in the study safely or to complete the scheduled study assessments, or that would confound the assessment of safety or efficacy.
9. The patient is aged 12 years or older and has a history of drug or alcohol use disorder within the past 1 year.
10. The patient has a history of suicide attempts within the past 1 year or is considered by the Investigator to be at imminent risk of suicide.
11. The patient has had a major surgical procedure within 30 days prior to screening.
12. The patient has had a DBS device implanted within the past 6 months.
13. The patient has an ANC of <1.5×10⁹/L at screening or at baseline.
14. In the opinion of the Investigator, the patient has abnormal laboratory values at screening or at baseline that are clinically significant and would compromise the safety of the patient during study participation.
15. The patient is pregnant or lactating, or presents with a positive serum pregnancy test at screening or a positive urine pregnancy test at baseline.
16. The patient is unwilling or, in the opinion of the Investigator, is unable to adhere to the requirements of the study, including study procedures and the visit schedule.

Patients who do not meet eligibility criteria may be re-screened up to 2 times, if the reasons for failing screening have resolved (eg, clinical laboratory values have normalized).

### 7.4 Investigational Product, Dose, and Mode of Administration

Compound (I) (1-(4-(6-chloropyridazin-3-yl)piperazin-1-yl)-2-(4-cyclopropyl-3-fluorophenyl)ethan-1-one) will be administered orally as small tablets (˜100 mg total weight, ≤6 mm), BID, using weight-based dosing determined by population PK analyses of data from the first-in-human Study (see Example 4), as well as data from this study (see STUDY DESIGN AND METHODOLOGY).

The reference treatment shall be placebo administered orally on the same schedule as Compound (I).

### 7.5 Study Duration

The study ends when the last randomized participant completes the study. The maximum duration of study participation is 92 weeks: up to 4 weeks for screening, 12 weeks for run-in, 72 weeks for the double-blind treatment period, and 4 weeks for safety follow-up after the last dose of study drug (the safety follow-up is not required if a participant enrolls in the separate open-label extension study).

### 7.6 Criteria for Evaluation

### Primary Efficacy Endpoint

The primary efficacy endpoint is change from baseline to Week 72 in the PKAN Activities of Daily Living (PKAN-ADL) score.

### Secondary Efficacy Endpoints

Secondary efficacy endpoints are change from baseline to Week 72 in the PKAN Disease Rating Scale (PKAN-DRS) score, and the value at Week 72 for Clinician Global Impression of Improvement (CGI-I).

### Safety Endpoints

The safety and tolerability of will be assessed throughout the study by monitoring the following:
Occurrence of treatment-emergent adverse events (TEAEs), classified according to their intensity (ie, mild, moderate, severe) or seriousness, and coded using the Medical Dictionary for Regulatory Activities (MedDRA) classification dictionary
Serial vital signs, weight, and physical examinations
Serial clinical laboratory testing (chemistry, hematology, coagulation)
Columbia Suicide Severity Rating Scale (C-SSRS; adult and pediatric versions)
Serial 12-lead electrocardiogram (ECG)

### Exploratory Efficacy Endpoints

Exploratory efficacy endpoints will include changes from baseline to Week 72 in the following:
Patient Global Impression of Improvement (PGI-I)
Quality of Life (QoL):
   Neuro-QoL, upper and lower extremity modules (adult and pediatric versions)
   EuroQol 5-dimension, 3-level QoL instrument (EQ-5D-3L) or EuroQol-5D Youth Version (EQ-5D-Y)
Functional independence: Functional Independence Measure (FIM) or Functional Independence Measure for Children (WeeFIM)
Function: diadochokinetic assessments of speech (ie, alternating motion rate, sequential motion rate) Pharmacokinetics

Multiple dose PK of Compound (I) in plasma will be evaluated using a sparse sampling design. Blood samples may also be used for metabolite identification or quantitation.

### Biomarkers

Exploratory biomarkers will be assessed in whole blood and plasma/serum.

### 7.7 Statistical Methods

### Analysis Sets:

Safety Population: All randomized participants who receive at least 1 dose of blinded study drug.

Full Analysis Set: All randomized participants with at least 1 post-baseline efficacy assessment.

### Demographics and Baseline Characteristics:

Demographic data, clinical characteristics, medical history, and prior treatments will be summarized for the safety population.

### Safety:

Safety will be evaluated based on TEAEs, vital signs, physical examinations, clinical laboratory assessments, C-SSRS assessments, and ECG findings.

### Pharmacokinetics:

Multiple-dose plasma concentrations of Compound (I) will be summarized.

Concentration measures may also be analyzed by nonlinear mixed-effects modeling, either alone or in conjunction with data from other studies.

### Efficacy:

For the primary efficacy endpoint (change from baseline to Week 72 in PKAN-ADL score), the treatment effect will be evaluated based on mixed model repeated measures (MMRM) analysis using an unstructured variance-covariance matrix. The model will include fixed effects for treatment, age (pediatric/adult), visit, and treatment-by-visit interaction, and baseline PKAN-ADL score as a covariate.

The secondary endpoints, change from baseline to Week 72 in PKAN-DRS score and CGI-I evaluated at Week 72, will be tested formally and hierarchically if p<0.05 on the primary endpoint is achieved. Change from baseline to Week 72 in PKAN-DRS score will be analyzed using a similar MMRM analysis as the primary endpoint. CGI-I will be analyzed with a chi-square test.

Treatment effects for exploratory efficacy endpoints that are continuous measures will be evaluated using a similar MMRM or fixed-effect analysis of covariance, as appropriate. Treatment effects for exploratory efficacy endpoints with categorical outcomes will be tested with chi-square methods.

### Biomarkers:

Biomarker assessments will be summarized with descriptive statistics and graphical displays.

### Power and Sample Size:

The sample size is calculated for the primary endpoint of change from baseline to Week 72 in PKAN-ADL. The calculation assumes that the mean difference between groups at Week 72 is 3 units and the within-group standard deviation for change from baseline is 4 units. To achieve 85% power with a two-sample t-test under 2:1 allocation and two-sided alpha=5% requires 72 participants completing the Week 72 visit. Assuming a drop-out rate of up to 14%, approximately 84 participants will be enrolled.

TABLE 15
Schedule of Study Events

| | Phase | | | | | | | | | | | | |
|---|---|---|---|---|---|---|---|---|---|---|---|---|---|
| | Screening^{(a)} | Run-in | | | | Double-Blind Treatment Period | | | | | | | |
| | Study Week | | | | | | | | | | | | |
| | −16 to −13 | −12 | −8 | −4 | −2 | 1 | | 2 | | 3 | | 4 | |
| | Study Day | | | | | | | | | | | | |
| | | −84 | −56 | −28 | −14 | 1 | 4 | 7 | 11 | 14 | 18 | 21 | 25 |
| | | Visit Window | | | | | | | | | | | |
| | −112 to −85 | ±3 | ±3 | ±3 | ±3 | | ±1 | ±1 | ±1 | ±1 | ±1 | ±1 | ±1 |
| Outpatient Clinic Visit | X | X | | X | | X | | | | X | | | |
| Home Health Visit | | | X | | X | | X | X | X | | X | X | X |
| Obtain informed consent/assent | X^{(c)} | | | | | | | | | | | | |
| Genetic testing for PKAN | X^{(c)} | | | | | | | | | | | | |
| Genotyping for CYP2C19 | X | | | | | | | | | | | | |
| Eligibility review | X | | | | | X | | | | | | | |
| Randomization | | X | | | | | | | | | | | |
| Demographics | X | | | | | | | | | | | | |
| Medical history | X | | | | | | | | | | | | |
| Weight/height | X | | | | | X | | | | X | | | |
| C-SSRS^{(d)} | X | | | | | X | | | | X | | | |
| Physical examination | X | | | | | X | | | | X | | | |
| Vital signs^{(e)} | X | X | X | X | X | X | X | X | X | X | X | X | X |
| 12-Lead ECG^{(e)} | X | | | | | X | | | | X | | | |
| Hematology | X | | | | | X | X | X | X | X | X | X | X |
| Chemistry | X | | | | | X | | | | X | | | |
| Serology | X | | | | | X | | | | | | | |
| Coagulation | X | | | | | X | | | | X | | | |
| PK blood sampling^{(f)} | | | | | | X | | X | | X | | X | |
| PD biomarker sampling^{(f)} | | X | | X | | X | | | | X | | | |
| Urinalysis | X | | | | | X | | | | X | | | |
| Serum/urine pregnancy^{(g)} | X | | | | | X | | | | X | | | |
| Urine drug/alcohol screen^{(h)} | X | | | | | X | | | | | | | |
| PKAN-DRS | | X | | X | | X | | | | | | | |
| CGI-I | | | | | | X | | | | | | | |
| Diadochokinetic assessments⁽ⁱ⁾ | | | | | | X | | | | | | | |
| FIM/WeeFIM | | | | | | X | | | | | | | |
| PKAN-ADL | | | X | | X | | | | | | | | |
| Neuro-QoL | | | X | | X | | | | | | | | |
| EQ-5D-3L/EQ-5D-Y | | | X | | X | | | | | | | | |
| PGI-I | | | X | | X | | | | | | | | |
| Study drug administration^{(j)} | | | | | | X | X | X | X | X | X | X | X |
| Review dosing diary | | | | | | X | X | X | X | X | X | X | X |

| | |
|---|---|
| Adverse events | [symbol] X [symbol] |
| Prior/concomitant medications | [symbol] X [symbol] |

| | Phase | | | | | | | |
|---|---|---|---|---|---|---|---|---|
| | Double-Blind Treatment Period | | | | | | | EOS^{(b)} |
| | Study Week | | | | | | | |
| | 6 | 12 | 24 | 36 | 48 | 60 | 72 | 76 |
| | Study Day | | | | | | | |
| | 42 | 84 | 168 | 252 | 546 | 420 | 504 | +30 |
| | Visit Window | | | | | | | |
| | ±3 | ±3 | ±3 | ±3 | ±3 | ±3 | ±3 | +7 |
| Outpatient Clinic Visit | X | | X | | X | | X | X |
| Home Health Visit | | X | | X | | X | | |
| Obtain informed consent/assent | | | | | | | | |
| Genetic testing for PKAN | | | | | | | | |
| Genotyping for CYP2C19 | | | | | | | | |
| Eligibility review | | | | | | | | |
| Randomization | | | | | | | | |
| Demographics | | | | | | | | |
| Medical history | | | | | | | | |
| Weight/height | X | | X | | X | | X | X |
| C-SSRS^{(d)} | X | | X | | X | | X | X |
| Physical examination | X | X | X | X | X | X | X | X |
| Vital signs^{(e)} | X | X | X | X | X | X | X | X |
| 12-Lead ECG^{(e)} | X | | X | | X | | X | X |
| Hematology | X | X | X | X | X | X | X | X |
| Chemistry | X | | X | | X | | X | X |
| Serology | | | | | | | | |
| Coagulation | X | | X | | X | | X | X |
| PK blood sampling^{(f)} | X | | X | | X | | X | |
| PD biomarker sampling^{(f)} | X | | X | | X | | X | X |
| Urinalysis | X | | X | | X | | X | X |
| Serum/urine pregnancy^{(g)} | X | X | X | X | X | X | X | X |
| Urine drug/alcohol screen^{(h)} | | | | | | | | |
| PKAN-DRS | X | | X | | X | | X | X |
| CGI-I | X | | X | | X | | X | X |
| Diadochokinetic assessments⁽ⁱ⁾ | X | | X | | X | | X | X |
| FIM/WeeFIM | | | | | | | X | X |
| PKAN-ADL | | X | | X | | X | X | X |
| Neuro-QoL | | X | | X | | X | X | X |
| EQ-5D-3L/EQ-5D-Y | | X | | X | | X | X | X |
| PGI-I | | X | | X | | X | X | X |
| Study drug administration^{(j)} | X | X | X | X | X | X | X | X |
| Review dosing diary | X | X | X | X | X | X | X | X |

| | |
|---|---|
| Adverse events | [symbol] X [symbol] |
| Prior/concomitant medications | [symbol] X [symbol] |

Abbreviations: CGI-I = clinician global impression of improvement; C-SSRS = Columbia Suicide Severity Rating Scale; ECG = electrocardiogram; EOS = end of study; EQ-5D-3L/EQ-5D-Y = EuroQol 5-dimension, 3-level quality of life instrument/EuroQol-5D Youth Version; FIM/WeeFIM = Functional Independence Measure/Functional Independence Measure for Children; Neuro-QoL = quality of life measure for adults/children with neurological disorders; PD = pharmacodynamic; PGI-I = patient global impression of improvement; PK = pharmacokinetic; PKAN-ADL = pantothenate kinase-associated neurodegeneration activities of daily living; PKAN-DRS = pantothenate kinase-associated neurodegeneration disease rating scale.
^{(a)}Patients who fail screening for any reason may be re-screened up to 2 times, if the reasons for failing screening have resolved (eg, clinical laboratory values have normalized).
^{(b)}Participants who are prematurely withdrawn from treatment but agree to continue the study will complete the EOS assessments as close as possible to the last dose of study drug. Participants who prematurely discontinue from the study will be contacted (via telephone call) 30 days (+7 days) after the last dose of study drug to ascertain participant safety. If a participant who has prematurely discontinued study drug decides to withdraw consent/assent and discontinue from the study within 6 weeks of completing the EOS Visit at the time of study drug withdrawal, then the EOS Visit may serve as the participant's final site visit. Participants who enroll in a separate open-label extension study will not be required to complete the EOS visit in this study.
^{(c)}Confirmation of mutation in the PANK2 gene may be obtained at a pre-screening visit before the screening visit. Obtain informed consent/assent before the test. The specific mutation will be recorded.
^{(d)}Participants will be administered the Screening/Baseline version of the C-SSRS at Screening and the Since Last Visit version of the C-SSRS at subsequent assessments.
^{(e)}At outpatient clinic visits, vital signs and 12-lead ECG will be assessed predose and 2 hours (±30 minutes) after the first dose of the day. Participants who have a deep brain stimulation device may experience interference with the ECG machine resulting in an inaccurate readout. In the best interest of the participant, the deep brain stimulation device should not be turned off to collect the ECG; rather, using clinical judgement and following consultation with the Sponsor Medical Monitor, ECG assessments are not required for these participants.
^{(f)}If possible, obtain PK blood sample predose and 1-2 hours after the dose. The actual time of administration of the 2 most recent doses of study drug and the exact times of sampling for PK and biomarker analysis will be recorded to the nearest minute on the eCRF.
^{(g)}To be assessed in women of childbearing potential only. A serum pregnancy test is required at baseline. Any positive urine pregnancy result at a subsequent visit will be confirmed by a serum pregnancy test.
^{(h)}To be conducted in participants 12 years of age and older. The Investigator should use clinical judgment to determine whether or not a participant with a positive test result should be admitted to the study (eg, if the Investigator believes that the result may be indicative of a drug or alcohol disorder, that the participant may not be able to comply with the protocol, or that participating in the study may put the participant at undue risk, the participant should be excluded per the exclusion criteria).
⁽ⁱ⁾Diadochokinetic assessments will include alternating motion rate and sequential motion rate. Sites will video record the assessments, which will be rated by a central rater.
^{(j)}Study drug will be taken orally twice daily, starting on Day 1 and will continue for up to 72 weeks. Study drug should be taken at approximately the same times cach day. Participants will be instructed to record the time they take study drug each day in a drug diary. Both the drug diary and the remaining study drug tablets should be brought with the participant to each clinic visit. Study drug should be administered with room temperature water.

Although the foregoing invention has been described in some detail by way of illustration and example for purposes of clarity of understanding, one of skill in the art will 10 appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A method of treating propionic acidemia (PA) and/or methylmalonic acidemia (MMA), comprising administering to a subject in need thereof a therapeutically effective amount of a compound represented by formula (I): 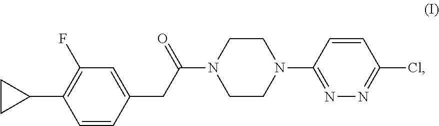 [SMILES: O=C(Cc1ccc(C2CC2)c(F)c1)N1CCN(c2ccc(Cl)nn2)CC1]
a pharmaceutically acceptable salt, hydrate, solvate, or a combination thereof.

2. The method of claim 1, wherein the subject has propionic acidemia (PA); and/or methylmalonic acidemia (MMA).

3. The method of claim 1 or 2, wherein the subject has one or more of 1) to 9):
1) an elevated 2-methylcitrate or 2-methylcitric acid (MCA) and/or an elevated MCA:citrate ratio at least in one of plasma, dried blood spot (DBS), and urine;
2) an elevated methylmalonate or methylmalonic acid (MMA) at least in one of plasma, dried blood spot (DBS), and urine;
3) an elevated 3-hydroxypropionate or 3-hydroxypropionic acid (3HP) at least in one of plasma, dried blood spot (DBS), and urine;
4) an elevated propionylglycine and/or tiglylglycine at least in one of plasma, dried blood spot (DBS), and urine;
5) an elevated propionylcarnitine (C3-carnitine), a reduced acetylcarnitine (C2-camitine), and/or an elevated C3:C2-camitine ratio at least in one of plasma, serum, and dried blood spot (DBS);
6) an elevated ammonium at least in one of plasma, serum, and dried blood spot (DBS);
7) an elevated Fibroblast growth factor 21 (FGF21) at least in one of plasma, serum, and dried blood spot (DBS);
8) an elevated pantothenate at least in one of plasma, serum, and dried blood spot (DBS); and
9) a reduced acetyl-CoA at least in one of plasma, serum, and dried blood spot (DBS).

4. The method of any one of claims 1 to 3, wherein the subject has one or more mutations in propionyl-CoA carboxylase alpha subunit (PCCA) and/or propionyl-CoA carboxylase beta subunit (PCCB).

5. The method of any one of claims 1 to 3, wherein the subject has a methylmalonyl-Coenzyme A mutase (MUT) deficiency.

6. The method of claim 5, wherein the MUT deficiency is caused by one or more changes or mutations in a MUT gene, which can cause no MUT enzyme (MUT0) or less than a normal amount of the MUT enzyme (MUT−) to be produced.

7. The method of any one of claims 1 to 6, wherein the subject has a normal level of vitamin B12 in serum or plasma and/or a normal level of homocysteine in plasma.

8. The method of any one of claims 1 to 7, wherein the subject does not have one or more conditions selected from the group consisting of:
a) alanine aminotransferase (ALT) and/or aspartate aminotransferase (AST) exceed two times of a upper limit of normal (ULN);
b) total bilirubin exceeds two times of a upper limit of normal (ULN);
c) an international normalized ratio (INR) is more than 1.4;
d) a baseline estimated glomerular filtration rate (eGFR) is less than 45 mL/min calculated using the CKD-EPI formula;
e) a positive test result for hepatitis B surface antigen, hepatitis C virus antibody, or HIV types 1 or 2 antibodies; and
f) a positive test result for SARS-CoV-2.

9. The method of any one of claims 1 to 8, wherein the treatment comprises one or more treatment cycles; and the administration of the compound of formula (I) comprises a dose escalation, a dose de-escalation, or no dose adjustment after a previous treatment cycle, wherein the dose escalation, dose de-escalation, or no dose adjustment is based at least in part on a safety assessment and/or an absolute neutrophil count (ANC).

10. The method of claim 9, wherein the administration of the compound of formula (I) comprises 1 to 6 dose escalations, optionally a dose de-escalation or no dose adjustment.

11. The method of claim 9 or 10, wherein:
a) when the ANC is less than 1.5×10⁹/L, but at or more than 1.0×10⁹/L, the administration of the compound of formula (I) is continued at the same dose of the previous treatment cycle, while monitoring the ANC till reaching at or more than 1.5×10⁹/L; or
b) when the ANC is less than 1.0×10⁹/L, but at or more than 0.5×10⁹/L, the administration of the compound of formula (I) is discontinued, while monitoring the ANC till reaching at or more than 1.5×10⁹/L; or
c) when the ANC is less than 0.5×10⁹/L confirmed by a second measurement on a second day, the administration of the compound of formula (I) is discontinued, while monitoring the ANC till reaching at or more than 1.5×10⁹/L.

12. The method of claim 11, wherein:
in a), the administration of the compound of formula (I) is not allowed for a dose escalation during next treatment cycles;
in b), when the ANC reaches at or more than 1.5×10⁹/L within about 14 days after discontinuation, the administration of the compound of formula (I) is resumed and comprises a dose de-escalation, provided that the previous treatment is a first treatment cycle, then the same dose is administered;
in c), when the ANC reaches at or more than 1.5×10⁹/L within about 3 days after discontinuation, the administration of the compound of formula (I) is resumed and comprises a dose de-escalation, provided that the previous treatment is a first treatment cycle, then the same dose is administered; or
in b) or c), when the ANC does not reach 1.5×10⁹/L within about 14 days after discontinuation, the administration of the compound of formula (I) is permanently discontinued.

13. A method of treating Pantothenate Kinase Associated Neurodegeneration (PKAN), comprising administering to a subject in need thereof a therapeutically effective amount of a compound represented by formula (I): 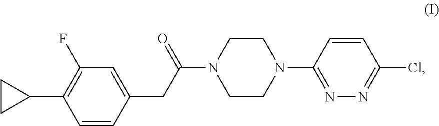 [SMILES: O=C(Cc1ccc(C2CC2)c(F)c1)N1CCN(c2ccc(Cl)nn2)CC1]
a pharmaceutically acceptable salt, hydrate, solvate, or a combination thereof.

14. The method of claim 13, wherein the subject has a diagnosis of PKAN as indicated by a confirmed mutation in the pantothenate kinase 2 gene (PANK2).

15. The method of claim 13 or 14, wherein the subject is at least 6 years of age, at least 12 years of age, at least 16 years of age, at least 18 years of age, 6-18 years old, or 6-12 years old.

16. The method of any one of claims 13 to 15, wherein the subject does not require ventilatory support.

17. The method of any one of claims 13 to 16, wherein the subject has not had a deep brain stimulation (DBS) device implanted within 6 months of initiating treatment with the compound; or is being treated with or has previously undergone treatment with deep brain stimulation (DBS).

18. The method of any one of claims 13 to 17, wherein the subject has an absolute neutrophil count (ANC) of less than 1.5×10⁹/L before initiating treatment with the compound.

19. The method of any one of claims 13 to 18, wherein the subject has not been treated with a strong or moderate CYP3A4 inhibitor or a strong CYP2C10 inhibitor before initiating treatment with the compound.

20. The method of any one of claims 13 to 19, wherein the subject has one or more symptoms selected from abnormality of extrapyramidal motor function, akinesia, muscular rigidity, chorea, athetosis, akathisia, abnormality of eye movement, acanthocytosis, attention deficit hyperactivity disorder (ADHD), behavioral abnormality, blindness, bulbar signs, Bull's eye maculopathy, choreoathetosis, craniofacial dystonia, dementia, depressivity, dysarthria, dystonia, emotional lability, tendency to fracture bones, gait disturbance, global developmental delay, hyperreflexia, impaired convergence, impulsivity, intellectual disability, intention or kinetic tremor, iron accumulation in globus pallidus and/or substantia nigra, limb dystonia, limb pain, loss of ability to walk, mental deterioration, neurological speech impairment, nyctalopia, obsessive-compulsive behavior, optic atrophy, osteopenia, palilalia, pallidal degeneration, parkinsonism, peripheral visual field loss, pigmentary retinopathy, psychotic mentation, retinal degeneration, retinal flecks, rigidity, rod-cone dystrophy, saccadic smooth pursuit, seizures, slurred speech, spasticity, speech articulation difficulties, tics, tip-toe gait, toe extensor amyotrophy, visual field defect, and visual impairment.

21. The method of claim 20, wherein the subject has one or more symptoms selected from abnormality of extrapyramidal motor function, choreoathetosis, dystonia, gait disturbance, loss of ability to walk, retinal degeneration, spasticity, and visual impairment.

22. The method of claim 20 or 21, wherein the subject has dystonia.

23. The method of claim 22, wherein the subject was diagnosed with dystonia before the age of 10.

24. The method of any one of claims 13 to 23, wherein the subject has abnormal levels of neurofilament light chain (NfL), glial fibraillary acid protein (GFAP), ubiquitin carboxyl-terminal hydrolase La (UCH-L1), and/or Tau in at least one of plasma, serum, and dried blood spot.

25. The method of claim 24, wherein the subject has abnormal levels of Tau in at least one of plasma, serum, and dried blood spot.

26. The method of claim 25, wherein the subject has abnormal levels of Tau in serum.

27. The method of any one of claims 13 to 26, wherein the subject has an elevated pantothenate at least in one of plasma, serum, and dried blood spot; and/or a reduced acetyl-CoA at least in one of plasma, serum, and dried blood spot.

28. The method of any one of claims 13 to 27, wherein the subject does not have one or more conditions selected from the group consisting of:
a) alanine aminotransferase (ALT) and/or aspartate aminotransferase (AST) exceed two times of a upper limit of normal (ULN);
b) total bilirubin exceeds two times of an upper limit of normal (ULN);
c) an international normalized ratio (INR) is more than 1.4;
d) a baseline estimated glomerular filtration rate (eGFR) is less than 45 mL/min calculated using the CKD-EPI formula;
e) a positive test result for hepatitis B surface antigen, hepatitis C virus antibody, or HIV types 1 or 2 antibodies; and
f) a positive test result for SARS-CoV-2.

29. The method of any one of claims 13 to 28, wherein the subject is taking or has previously taken a therapy selected from fosmetpantotenate, pantothenate (e.g., calcium pantothenate), deferiprone, baclofen, a muscle relaxant (anticholinergics, benzodiazepines, and other anti-spasticity agents), and/or a treatment for Parkinson's disease.

30. The method of any one of claims 13 to 29, wherein the subject has previously undergone a surgical ablation procedure.

31. The method of claim 30, wherein the subject has previously undergone a thalamotomy or pallidotomy.

32. The method of any one of claims 13 to 31, wherein the treatment comprises one or more treatment cycles; and the administration of the compound of formula (I) comprises a dose escalation, a dose de-escalation, or no dose adjustment after a previous treatment cycle, wherein the dose escalation, dose de-escalation, or no dose adjustment is based at least in part on a safety assessment and/or an absolute neutrophil count (ANC).

33. The method of claim 32, wherein:
a) when the ANC is less than 1.5×10⁹/L, but at or more than 1.0×10⁹/L, the administration of the compound of formula (I) is continued at the same dose of the previous treatment cycle;
b) when the ANC is less than 1.0×10⁹/L, but at or more than 0.5×10⁹/L, the administration of the compound of formula (I) is at least temporarily discontinued until the ANC increases to at least 1.5×10⁹/L; and/or
c) when the ANC is less than 0.5×10⁹/L, the administration of the compound of formula (I) is at least temporarily discontinued.

34. The method of claim 33, wherein:
in a), the administration of the compound of formula (I) continues at the same dose of the previous treatment cycle in the subsequent treatment cycle;
in b), (i) when the ANC has increased to at least 1.5×10⁹/L within about 14 days after discontinuation, the administration of the compound of formula (I) is resumed; and (ii) when the ANC has not increased to at least 0.5×10⁹/L within about 14 days after discontinuation, the administration of the compound of formula (I) remains at least temporarily discontinued; or
in c), (i) when the ANC has increased to at least 1.5×10⁹/L within about 3 days after discontinuation, the administration of the compound of formula (I) is resumed and comprises a dose de-escalation; and (ii) when the ANC has not increased to at least 0.5 12×10⁹/L within about 14 days after discontinuation, the administration of the compound of formula (I) remains at least temporarily discontinued.

35. The method of any one of claims 1 to 34, wherein the therapeutically effective amount is a total daily dosage of from about 1 mg to about 500 mg, from about 2 mg to about 500 mg, from about 3 mg to about 500 mg, from about 5 mg to about 500 mg, from about 10 mg to about 500 mg, from about 20 mg to about 500 mg, from about 30 mg to about 500 mg, from about 40 mg to about 500 mg, from about 50 mg to about 500 mg, from about 60 mg to about 500 mg, from about 70 mg to about 500 mg, from about 80 mg to about 500 mg, from about 90 mg to about 500 mg, from about 100 mg to about 500 mg, from about 10 mg to about 300 mg, from about 20 mg to about 300 mg, from about 30 mg to about 300 mg, from about 40 mg to about 300 mg, from about 50 mg to about 300 mg, from about 60 mg to about 300 mg, from about 70 mg to about 300 mg, from about 80 mg to about 300 mg, from about 90 mg to about 300 mg, from about 100 mg to about 300 mg, from about 10 mg to about 200 mg, from about 20 mg to about 200 mg, from about 30 mg to about 200 mg, from about 40 mg to about 200 mg, from about 50 mg to about 200 mg, from about 60 mg to about 200 mg, from about 70 mg to about 200 mg, from about 80 mg to about 200 mg, from about 90 mg to about 200 mg, from about 100 mg to about 200 mg, from about 10 mg to about 120 mg, from about 20 mg to about 120 mg, from about 30 mg to about 120 mg, from about 40 mg to about 120 mg, from about 50 mg to about 120 mg, from about 60 mg to about 120 mg, from about 70 mg to about 120 mg, from about 80 mg to about 120 mg, from about 90 mg to about 120 mg, from about 100 mg to about 120 mg, from about 10 mg to about 100 mg, from about 20 mg to about 100 mg, from about 30 mg to about 100 mg, from about 40 mg to about 100 mg, from about 50 mg to about 100 mg, from about 60 mg to about 100 mg, from about 70 mg to about 100 mg, from about 80 mg to about 100 mg, or from about 90 mg to about 100 mg of the compound of formula (I).

36. The method of claim 35, wherein the therapeutically effective amount is a total daily dosage of from about 10 mg to about 120 mg, from about 20 mg to about 120 mg, from about 30 mg to about 120 mg, from about 40 mg to about 120 mg, from about 50 mg to about 120 mg, from about 60 mg to about 120 mg, from about 70 mg to about 120 mg, from about 80 mg to about 120 mg, from about 90 mg to about 120 mg, from about 100 mg to about 120 mg, from about 10 mg to about 100 mg, from about 20 mg to about 100 mg, from about 30 mg to about 100 mg, from about 40 mg to about 100 mg, from about 50 mg to about 100 mg, from about 60 mg to about 100 mg, from about 70 mg to about 100 mg, from about 80 mg to about 100 mg, or from about 90 mg to about 100 mg of the compound of formula (I).

37. The method of claim 36, wherein the therapeutically effective amount is a total daily dosage of from about 50 mg to about 120 mg, from about 60 mg to about 120 mg, from about 70 mg to about 120 mg, from about 80 mg to about 120 mg, from about 90 mg to about 120 mg, from about 100 mg to about 120 mg, from about 50 mg to about 100 mg, from about 60 mg to about 100 mg, from about 70 mg to about 100 mg, from about 80 mg to about 100 mg, or from about 90 mg to about 100 mg of the compound of formula (I).

38. The method of claim 37, wherein the therapeutically effective amount is a total daily dosage of about 10 mg, about 20 mg, about 30 mg, about 40 mg, about 50 mg, about 60 mg, about 70 mg, about 80 mg, about 90 mg, about 100 mg, about 110 mg, or about 120 mg of the compound of formula (I).

39. The method of any one of claims 1 to 38, wherein the compound of formula (I) is administered orally.

40. The method of any one of claims 1 to 39, wherein the compound of formula (I) is administered once, twice, three times, or four times daily.

41. The method of claim 40, wherein the compound of formula (I) is administered once daily.

42. The method of claim 40, wherein the compound of formula (I) is administered twice daily.

43. The method of claim 40 or 42, wherein the compound of formula (I) is administered twice daily in at least about 10 hours apart.

44. The method of any one of claims 1 to 43, wherein the compound of formula (I) is administered to the subject under a fasted condition, wherein the subject has no food consumption except for water at least about 10 hours prior to the administration and at least about 4 hours post the administration.

45. The method of any one of claims 1 to 43, wherein the compound of formula (I) is administered to the subject under a fed condition, wherein the subject has no food consumption except for water at least about 10 hours prior to the administration; and the compound of formula (I) is administered right after food.

46. The method of any one of claims 1 to 43, wherein the compound of formula (I) is administered under a hybrid condition comprising a fasted condition according to claim 44 and a fed condition according to claim 45.

47. The method of any one of claims 1 to 12 and 35 to 46, wherein the subject is further evaluated according to one or more parameters of Table 6, Table 7, Table 8, and Table 9 comprising plasma pharmacokinetic parameters and/or pharmacodynamic parameters.

48. The method of any one of claims 13 to 46, wherein the subject is further evaluated according to one or more parameters of Table 6, Table 7, Table 8, Table 9, and Table 15 comprising plasma pharmacokinetic parameters and/or pharmacodynamic parameters.

49. The method of any one of claims 1 to 48, wherein the subject is further evaluated for one or more biomarkers comprising whole blood acetyl-CoA and/or plasma pantothenate.


SEQUENCE LISTING (27 sequences)

SEQ ID NO: 1 - DNA, 23 bp, synthetic construct
  source 1..23: mol_type = other DNA
aggaagccaggaaaggttatttg

SEQ ID NO: 2 - DNA, 21 bp, synthetic construct
  source 1..21: mol_type = other DNA
ccctacaggcaatttctcctc

SEQ ID NO: 3 - DNA, 20 bp, synthetic construct
  source 1..20: mol_type = other DNA
ggatgatctggacgaagagc

SEQ ID NO: 4 - DNA, 20 bp, synthetic construct
  source 1..20: mol_type = other DNA
acagtgcttaatggtgtccc

SEQ ID NO: 5 - DNA, 22 bp, synthetic construct
  source 1..22: mol_type = other DNA
cttctgcatccttgactactcc

SEQ ID NO: 6 - DNA, 20 bp, synthetic construct
  source 1..20: mol_type = other DNA
caaatgttgcttgtctggtg

SEQ ID NO: 7 - DNA, 20 bp, synthetic construct
  source 1..20: mol_type = other DNA
gtcagtcgagtgcacagttt

SEQ ID NO: 8 - DNA, 20 bp, synthetic construct
  source 1..20: mol_type = other DNA
cacatgaagcagcacgactt

SEQ ID NO: 9 - DNA, 20 bp, synthetic construct
  source 1..20: mol_type = other DNA
tgctcaggtagtggttgtcg

SEQ ID NO: 10 - DNA, 20 bp, synthetic construct
  source 1..20: mol_type = other DNA
caaatgttgcttgtctggtg

SEQ ID NO: 11 - DNA, 20 bp, synthetic construct
  source 1..20: mol_type = other DNA
gtcagtcgagtgcacagttt

SEQ ID NO: 12 - DNA, 22 bp, synthetic construct
  source 1..22: mol_type = other DNA
ggataggatggctactagctgc

SEQ ID NO: 13 - skipped

SEQ ID NO: 14 - DNA, 21 bp, synthetic construct
  source 1..21: mol_type = other DNA
gcctaatttcgttcacagtgg

SEQ ID NO: 15 - DNA, 24 bp, synthetic construct
  source 1..24: mol_type = other DNA
ctggaaatctcgtgtagttgagtc

SEQ ID NO: 16 - DNA, 23 bp, synthetic construct
  source 1..23: mol_type = other DNA
tcaagcagtatcaaagacaccac

SEQ ID NO: 17 - DNA, 22 bp, synthetic construct
  source 1..22: mol_type = other DNA
cactggagcagtaactgagagc

SEQ ID NO: 18 - DNA, 19 bp, synthetic construct
  source 1..19: mol_type = other DNA
ctcagcgctgcctcagtct

SEQ ID NO: 19 - DNA, 18 bp, synthetic construct
  source 1..18: mol_type = other DNA
gcatcgaccggtaatgca

SEQ ID NO: 20 - DNA, 20 bp, synthetic construct
  source 1..20: mol_type = other DNA
caaatgttgcttgtctggtg

SEQ ID NO: 21 - DNA, 20 bp, synthetic construct
  source 1..20: mol_type = other DNA
gtcagtcgagtgcacagttt

SEQ ID NO: 22 - DNA, 20 bp, synthetic construct
  source 1..20: mol_type = other DNA
tcagggccttctttacgttg

SEQ ID NO: 23 - DNA, 20 bp, synthetic construct
  source 1..20: mol_type = other DNA
tggagtacactgccaggatg

SEQ ID NO: 24 - DNA, 21 bp, synthetic construct
  source 1..21: mol_type = other DNA
ccctgctgattctgaaaaatg

SEQ ID NO: 25 - DNA, 20 bp, synthetic construct
  source 1..20: mol_type = other DNA
gagccgatgttcaccagaag

SEQ ID NO: 26 - DNA, 20 bp, synthetic construct
  source 1..20: mol_type = other DNA
aacctccacctgcacaaact

SEQ ID NO: 27 - DNA, 20 bp, synthetic construct
  source 1..20: mol_type = other DNA
tgggtaaggatcatccaggt